SEQUENCE LISTING (22 sequences)

SEQ ID NO: 1 - DNA, 395 bp, Dictyostelium discoideum AX4
atgacaattttatgtaagtaaaatatttaatgattaaatttaatagttttcaaattagaatttatttatttattaacatttttttttttttttttttttttttttttaaaattaaatttcactttattttaaaaagcaacctttacatcattttcaaatccacccaaattaaataaatcttcattttcatcatcaacgggttcatcattatcaatgggatcaaattcatttgcatgggggggaggttgggggggttttgggggcccaaaagggggaagttttaatgtggacattgctggaaatttaatttggggggtttattagggggtttataagggggggagtgggactggttaagtggagagggctccaaaagggatgcaagcagcgcctga

SEQ ID NO: 2 - protein, 88 aa, Dictyostelium discoideum AX4
MTILSTFTSFSNPPKLNKSSFSSSTGSSLSMGSNSFAWGGGWGGFGGPKGGSFNVDIAGNLIWGVYGFIRGGVGLVKWRGLQKGCKQP

SEQ ID NO: 3 - protein, 88 aa, Artificial Sequence
  synthetic
MTILSTFTSFSNPPKLNKSSFSSSTGSSLSMGSNSFAWGGGWGGFGGPKGGSFNVDIAGNAAAAAAAAAAGGVGLVKWRGLQKGCKQP

SEQ ID NO: 4 - protein, 88 aa, Artificial Sequence
  synthetic
MTILSTFTSFSNPPKLNKSSFSSSTGSSLSMGSNSFAWGGGWGGFGGPKGGSFNVDIAGNLIWGVYGFIRAAAAAAAAAALQKGCKQP

SEQ ID NO: 5 - protein, 10 aa, Artificial Sequence
  synthetic
LIWGVYGFIR

SEQ ID NO: 6 - protein, 88 aa, Artificial Sequence
  synthetic
MAILAAFAAFANPPKLNKAAFAAAAGAALAMGANAFAWGGGWGGFGGPKGGSFNVDIAGNLIWGVYGFIRGGVGLVKWRGLQKGCKQP

SEQ ID NO: 7 - protein, 10 aa, Artificial Sequence
  synthetic
GGVGLVKWRG

SEQ ID NO: 8 - protein, 38 aa, Artificial Sequence
  synthetic
MTILSTFTSFSNPPKLNKSSFSSSTGSSLSMGSNSFAW

SEQ ID NO: 9 - protein, 20 aa, Artificial Sequence
  synthetic
LIWGVYGFIRGGVGLVKWRG

SEQ ID NO: 10 - protein, 22 aa, Artificial Sequence
  synthetic
  DISULFID (1)..(22)
CLIWGVYGFIRGGVGLVKWRGC

SEQ ID NO: 11 - protein, 20 aa, Artificial Sequence
  synthetic
LIWGVYGFIRAAAAAAAAAA

SEQ ID NO: 12 - protein, 38 aa, Artificial Sequence; identical to SEQ ID NO: 8
  synthetic

SEQ ID NO: 13 - protein, 38 aa, Artificial Sequence
  synthetic
MAILAAFAAFANPPKLNKAAFAAAAGAALAMGANAFAW

SEQ ID NO: 14 - protein, 49 aa, Artificial Sequence
  synthetic
GGWGGFGGPKGGSFNVDIAGNLIWGVYGFIRGGVGLVKWRGLQKGCKQP

SEQ ID NO: 15 - protein, 24 aa, Artificial Sequence
  synthetic
EQVTSVGGAVVTGVTAVAQKTEGA

SEQ ID NO: 16 - protein, 21 aa, Artificial Sequence
  synthetic
GAAAGAVVGGLGGYMLGSAMS

SEQ ID NO: 17 - protein, 41 aa, Artificial Sequence
  synthetic
DEAFRHDSGYEVHHQKLVFFAEDVGSNKAIIGLMVGGVVIA

SEQ ID NO: 18 - DNA, 21 bp, Artificial Sequence
  synthetic
cattcacaggtaatcatgcta

SEQ ID NO: 19 - protein, 7 aa, Artificial Sequence
  synthetic
HSQVIML

SEQ ID NO: 20 - DNA, 21 bp, Artificial Sequence
  synthetic
cattcacagcagcaggtaatc

SEQ ID NO: 21 - protein, 7 aa, Artificial Sequence
  synthetic
HSQQQVI

SEQ ID NO: 22 - DNA, 27 bp, Artificial Sequence
  synthetic
cagcagcagcagcagcagcagcagcag

(19) United States
(12) Patent Application Publication
(10) Pub. No.: US 2020/0207835 A1
(43) Pub. Date: Jul. 2, 2020

(54) SRCP1-BASED THERAPY FOR DISEASES ASSOCIATED WITH PROTEIN AGGREGATION

(71) Applicant: THE MEDICAL COLLEGE OF WISCONSIN, INC., Milwaukee, MI (US)

(72) Inventor: Kenneth Matthew Scaglione, Milwaukee, WI (US)

(73) Assignee: THE MEDICAL COLLEGE OF WISCONSIN, INC., Milwaukee, WI (US)

(21) Appl. No.: 16/639,727

(22) PCT Filed: Aug. 17, 2018
(86) PCT No.: PCT/US18/46978

§ 371 Date: Feb. 17, 2020

Related U.S. Application Data
(60) Provisional application No. 62/547,334, filed on Aug. 18, 2017.
(60) Provisional application No. 62/671,708, filed on May 15, 2018.

(51) Int. Cl. C07K 14/71; C12N 15/86

(52) U.S. Cl. CPC C07K 14/71; A61K 38/00; C12N 15/86

(57) ABSTRACT

Compositions, methods and kits for the reduction of protein aggregation are provided. Further compositions, methods and kits are also provided for the treatment of diseases associated with protein aggregation, particularly polyglutamine disease, including Huntington's disease.

### CROSS-REFERENCE TO RELATED APPLICATIONS

This application claims priority to U.S. Provisional Application No. 62/547,334 filed on Aug. 18, 2017 and U.S. Provisional Application No. 62/671,708, filed on May 15, 2018, the contents of each are incorporated by reference in their entireties.

### STATEMENT REGARDING FEDERALLY SPONSORED RESEARCH

This invention was made with government support under R35 GM119544 awarded by National Institute of General Medical Sciences. The government has certain rights in the invention.

### BACKGROUND OF THE INVENTION

Protein aggregation is a biological phenomenon in which impaired homeostatic mechanisms lead to the accumulation of proteins in the cell. This can lead to cell death and other pathologies. The phenomenon is associated with more than 71 diseases, including Huntington's disease. One such group of protein aggregation diseases, polyglutamine diseases, includes a group of nine neurodegenerative diseases caused by the presence of an expanded polyglutamine repeat in specific proteins. Polyglutamine expansion leads to protein aggregation that ultimately results in loss of specific types on neurons, and eventually death. Polyglutamine aggregation is thought to be a key early event in polyglutamine toxicity, and suppression of polyglutamine aggregation is one way to potentially treat these diseases.

Included in the polyglutamine diseases is Huntington's disease. People with Huntington's disease have a mutation in the gene that codes for the protein called Huntingtin. When this gene is translated, the resulting protein has longer tracts of glutamines. These polyglutamine tracts are sticky, so the proteins are prone to aggregation and accumulate in nuclei of neurons, disrupting the normal function of these cells.

There is no way to prevent or treat these diseases at this time. Available treatments focus solely on improving symptoms.

Polyglutamine aggregation has been studied in a wide variety of organisms ranging from yeast to primates. In each case expression of a polyglutamine expanded protein results in the formation of protein aggregates, with the exception of one organism, Dictyostelium discoideum. Dictyostelium discoideum encodes a unique genome among sequenced organisms, in that it encodes large amounts of homopolymeric amino acid tracts. Among the most common homopolymeric amino acid repeats are polyglutamine repeats of 10 glutamines or more. Endogenous polyglutamine tracts in Dictyostelium reach well beyond the disease threshold reaching repeats lengths of beyond 80 glutamines.

### SUMMARY OF THE INVENTION

The inventors have discovered the protein responsible for the resistance to polyglutamine aggregation, and have shown that this protein can be used to reduce polyglutamine aggregation in cells, which can be used for the treatment of polyglutamine diseases. Specifically, an isolated peptide of 10 amino acids (SEQ ID NO:5) is able to reduce aggregation in vitro. Suitable compositions, peptides, and kits comprising the inhibitor of protein aggregation are provided herein.

The present disclosure describes a novel chaperone protein of Dictyostelium, SRCP1, that can suppress protein aggregation, including polyglutamine aggregation, and include peptides, vectors, viruses, and compositions comprising the SRCP1 protein, modified proteins thereof, and peptides and fragments thereof. Further, compositions comprising the peptides and fragments thereof can be used for methods of inhibiting protein aggregation, including polyglutamine aggregation, and treating diseases associated with protein aggregation, such as, but not limited to, polyglutamine diseases, including Huntington's disease.

In one aspect, the disclosure provides an isolated peptide inhibitor of protein aggregation comprising SEQ ID NO:5 (LIWGVYGFIR). In another aspect, the disclosure provides an isolated peptide comprising SEQ ID NO:9 (LIWGVYGFIRGGVGLVKWRG).

In another aspect, the disclosure provides an isolated peptide inhibitor of protein aggregation comprising SEQ ID NO:5 and 1-78 additional amino acids selected from amino acids 1-60 or 71-88 of SEQ ID NO:2. In some aspects, the additional amino acids selected from amino acids 1-60 or 71-88 of SEQ ID NO:2 have at least one amino acid mutation within amino acids 1-60 or 71-88 from the sequence found in SEQ ID NO:2.

In another aspect, the disclosure provides an isolated peptide wherein the peptide has at least 40% sequence similarity with SEQ ID NO:2 and wherein amino acids 61-70 are SEQ ID NO:5. In another aspect, the isolated peptide has at least 60% sequence similarity with SEQ ID NO:2 and wherein amino acids 61-70 are SEQ ID NO:5. In yet another aspect, the isolated peptide has at least 80% sequence similarity with SEQ ID NO:2 and wherein amino acids 61-70 are SEQ ID NO:5. In yet a further aspect, the peptide has at least 90% sequence similarity with SEQ ID NO:2 and wherein amino acids 61-70 are SEQ ID NO:5.

In some aspects, the isolated peptide or proteins are directly or indirectly linked to a tag or agent.

In a further aspect, the present disclosure provides a vector able to express the isolated peptide or proteins described herein. In another aspect, the disclosure provides a vector comprising (a) a polynucleotide sequence encoding the isolated peptide described herein and (b) a heterologous polynucleotide sequence. In some aspects, the heterologous sequence is a tag or agent.

In further aspects, the present disclosure provides a pharmaceutical composition comprising the isolated peptide inhibitor of protein aggregation comprising SEQ ID NO:5 and a pharmaceutically acceptable carrier.

In yet another aspect, the disclosure provides a method of suppressing, reducing or inhibiting protein aggregation in a host cell, the method comprising: (a) introducing within the host cell an effective amount of (i) an isolated peptide described herein, (ii) a vector able to express the isolated peptide described herein, (iii) a virus capable of expressing the isolated peptide in a cell, or (iv) a composition described herein, wherein the protein aggregation within the host cell is suppressed, reduced or inhibited.

In another aspect, the present disclosure provides a method of treating a polyglutamine disease in a subject in need thereof, the method comprising the steps of: (a) administering to the subject one of the following (i) an isolated peptide described herein, (ii) a vector of able to express the isolated peptide described herein, (iii) a virus capable of expressing the isolated peptide in a cell, or (iv) a composition described herein, in an effective amount to reduce, inhibit or prevent at least one symptom of the polyglutamine disease associated with aggregation of the polyglutamine protein.

In yet another aspect, the present disclosure provides method of treating a disease associated with aggregation of a protein in a subject in need thereof, the method comprising the steps of: (a) administering to the subject (i) an isolated peptide described herein, (ii) a vector able to express the isolated peptide described herein, (iii) a virus capable of expressing the isolated peptide in a cell, or (iv) a composition described herein in an effective amount to reduce, inhibit or prevent at least one symptom of the disease associated with aggregation.

In yet another aspect, a vector able to express SRCP1 protein or a modified SRCP1 protein is provided. In one aspect, the SRCP1 protein is SEQ ID NO:2, or an amino acid sequence with at least 80% sequence identity to SEQ ID NO:2, preferably at least 90% or at least 95% sequence identity to SEQ ID NO:2.

In another aspect, a modified protein of SRCP1 comprising at least one amino acid mutation from SEQ ID NO:2 and wherein amino acids 61-70 is SEQ ID NO:5. In one aspect the modified protein is SEQ ID NO:4 or SEQ ID NO:6.

In yet another aspect, a vector comprising (a) a nucleic acid sequence encoding a protein selected from the group consisting of: (i) SRCP1 protein of SEQ ID NO: 2, (ii) a fragment of the SRCP1 protein comprising at least amino acids 61-70 of SEQ ID NO: 2; (iii) a peptide of SRCP1 comprising SEQ ID NO:5; (iv) a peptide of SEQ ID NO:5, (v) a modified protein of SRCP1 comprising at least one amino acid mutation from SEQ ID NO:2, (vi) a modified protein of SEQ ID NO:4, (vii) a modified protein of SEQ ID NO:6; (viii) a modified SRCP1 protein comprising SEQ ID NO:9, (ix) a modified SCRP1 protein comprising SEQ ID NO:10, and (b) a heterologous nucleic acid sequence, wherein the vector expresses the protein, peptide or modified protein in a host cell is provided.

In yet another aspect, a composition comprising (i) SRCP1 protein of SEQ ID NO:2; (ii) a modified SRCP1 protein of SEQ ID NO:2 comprising at least one amino acid mutation and wherein amino acids 61-70 is SEQ ID NO:5; (iii) a SRCP1 peptide comprising SEQ ID NO:5; (iv) a vector able to express (i), (ii) or (iii); (v) a virus able to direct expression of (i), (ii) or (iii) in a cell. In some embodiments, the composition further comprises a pharmaceutically acceptable carrier.

Further aspects provide a method of reducing or inhibiting polyglutamine aggregation in a host cell, the method comprising (a) introducing in an effective amount within the host cell (i) the SRCP1 protein of SEQ ID NO: 2; (ii) a fragment of the SRCP1 protein comprising at least amino acids 61-70 of SEQ ID NO: 2; (iii) a modified SRCP1 protein; (iv) a peptide comprising SEQ ID NO:5, (iv) a vector able to express (i)-(iv); or (v) a virus able to direct expression of (i)-(iii).

Another aspect provides a method of treating a polyglutamine disease in a subject in need thereof, the method comprising the steps of: (a) administering to the subject (i) the SRCP1 protein of SEQ ID NO: 2, (ii) a fragment of the SRCP1 protein comprising at least amino acids 61-70 of SEQ ID NO: 2; (iii) a modified SRCP1 protein comprising SEQ ID NO:2 with at least one mutation of one amino acid; (iv) a peptide comprising SEQ ID NO:5, (v) a modified protein of SRCP1 comprising at least SEQ ID NO:5, (vi) the vector encoding and able to express the protein of any one (i)-(v); (vi) a virus able to express the protein of any one of (i)-(v); or (viii) the composition comprising the protein of any one of (i)-(v), the vector of (vi) or the virus of (vii) in an effective amount to reduce, inhibit or prevent at least one symptom of the polyglutamine disease associated with aggregation of the polyglutamine protein.

In one aspect, the methods provided herein are able to treat Huntington's disease. In one embodiment, the methods are able to reduce the amount of polyglutamine aggregated huntintin protein in a host cell.

Further aspects provide a method of reducing or inhibiting protein aggregation in a host cell, the method comprising (a) introducing in an effective amount within the host cell (i) the SRCP1 protein of SEQ ID NO: 2, (ii) a fragment of the SRCP1 protein of SEQ ID NO:2 comprising at least amino acids 61-70 of SEQ ID NO: 2; (iii) a peptide of SEQ ID NO:5, (iv) a modified SRCP1 protein described herein, (v) a vector able to express (i)-(iv), or (vi) a virus able to direct expression of (i)-(iv). Suitably, the protein aggregation is associated with a neurodegenerative disease, for example, but not limited to, huntingtin protein, SOD-1, and α-synuclein.

Another aspect provides a method of treating a disease associated with protein aggregation in a subject in need thereof, the method comprising the steps of: (a) administering to the subject (i) a SRCP1 protein or modified SRCP1 protein selected from the group consisting of: (a) SRCP1 protein of SEQ ID NO: 2, (b) a fragment of the SRCP1 protein comprising at least amino acids 61-70 of SEQ ID NO: 2, (c) a modified SRCP1 protein comprising at least one mutation in SEQ ID NO:2 and containing at least SEQ ID NO:5 at amino acids 61-70 of SEQ ID NO:2, (d) a peptide of SEQ ID NO:5, and (e) modified SRCP1 protein described herein; (ii) a vector able to express (1) SRCP1 protein of SEQ ID NO: 2, (2) a fragment of the SRCP1 protein comprising at least amino acids 61-70 of SEQ ID NO: 2; (3) a peptide of SEQ ID NO:5, and (4) a modified protein of SRCP1; (iii) a virus able to express (1)-(4), or (iii) a composition comprising (1) SRCP1 protein of SEQ ID NO: 2, (2) a fragment of the SRCP1 protein comprising at least amino acids 61-70 of SEQ ID NO: 2; (3) a peptide of SEQ ID NO:5, and (4) a modified protein of SRCP1, in an effective amount to reduce, inhibit or prevent at least one symptom of the disease associated with aggregation of the target protein.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J demonstrates known protein quality control pathways do not protect against ^{GFP}Htt^{ex1Q103} aggregation. (A,B) Proteasome inhibition does not result in an accumulation of aggregated ^{GFP}Htt^{ex1Q103}. Either vehicle (DMSO) (A) or 100 μM MG132 (B) was added to Dictyostelium expressing ^{GFP}Htt^{ex1Q103} for 18 hours prior to imaging GFP fluorescence. Shown is a representative image (n=3). (C,D) Inhibition of Hsp70 does not lead to accumulation of aggregated ^{GFP}Htt^{ex1Q103}. Either DMSO (C) or 80 μM VER-155008 (D) was added to Dictyostelium expressing ^{GFP}Htt^{ex1Q103} for 24 hours prior to imaging GFP fluorescence. Shown is a representative image (n=3). (E,F) Inhibition of autophagy does not lead to accumulation of aggregated ^{GFP}Htt^{ex1Q103}. Dictyostelium expressing ^{GFP}Htt^{ex1Q103} were either untreated (E) or treated with 150 mM NH₄Cl (F) for 8 hours prior to imaging GFP fluorescence. Shown is a representative image (n=3). (G) Proteasome inhibitor MG132 leads to an accumulation of ubiquitinated species Dictyostelium. Wildtype cells expressing ^{GFP}Htt^{ex1Q103} were treated with increasing concentrations of MG132 for 18 hours. Cells were then collected and analyzed by western blot for ubiquitin. A representative image is shown (n=3). (H) Hsp70 inhibitor Ver155008 leads to an increase of ATG8 proteins levels. Wildtype cells were treated with either DMSO or 80 μM Ver155008 for 24 hours. Cells were then collected and analyzed by western blot for ATG8. (I) Quantification of the levels of ATG8 protein levels from (H). Levels of ATG8 protein were quantified using ImageJ (n=3, * p<0.05). Error bars indicate SD. (J) Lysosome inhibitor NH₄Cl leads to an accumulation of large ATG8 puncta. Wildtype cells were either untreated or treated with 150 mM NH₄Cl for 8 hours. Cells were then fixed and immunostained for ATG8. A representative image is shown (n=3).

FIGS. 2A-2O demonstrate the identification of a novel protein that suppresses polyQ aggregation in Dictyostelium. (A-C) A REMI screen identifies clones with ^{GFP}Htt^{ex1Q103} aggregates. A REMI screen was performed in Dictyostelium expressing ^{GFP}Htt^{ex1Q103}, and clonal isolates were plated in 96-well plates prior to analysis by high-content imaging. Shown are representative negative (A) and positive (B, C) hits from the REMI screen. (D) Quantification of the number of cells with ^{GFP}Htt^{ex1Q103} puncta from (A-C) (n=4, **** p<0.0001). Error bars indicate SD. (E-G) Deletion of SRCP1 results in ^{GFP}Htt^{ex1Q103} aggregation in Dictyostelium. SRCP1 knockout cells were generated by homologous recombination and selected with blasticidin. ^{GFP}Htt^{ex1Q103} was electroporated into wild-type (E) or SRCP1 knockout cells (F, G), selected with G-418, and imaged by fluorescent microscopy. (H) Quantification of cells with ^{GFP}Htt^{ex1Q103} puncta from SRCP1+/+ and SRCP1−/− cell lines (n=4, * p<0.05). Error bars indicate SD. (I) Deletion of SRCP1 results in the accumulation of aggregated ^{GFP}Htt^{ex1Q103}. Wildtype or SRCP1 knockout cells expressing ^{GFP}Htt^{ex1Q103} were lysed in NETN buffer and quantified by BCA protein assay. Samples were prepared with SDS and 40 μg of protein were used for filter trap assay. A representative image is shown (n=3). (J) Quantification of ^{GFP}Htt^{ex1Q103} present in the filter trap assay (I). The amount of ^{GFP}Htt^{ex1Q103} present in (I) was quantified using ImageJ (n=3, ** p<0.01). Error bars indicate SD. (K-L) SRCP1 expression rescues ^{GFP}Htt^{ex1Q103} aggregation in SRCP1 knockout cells. Wild-type and SRCP1 knockout cells were transfected with either RFP alone (K) or ^{RFP}SRCP1 (L) and selected with hygromycin B. Cells were then subsequently transfected with ^{GFP}Htt^{ex1Q103}, selected with G-418, and imaged by fluorescent microscopy. (M) Quantification of cells with ^{GFP}Htt^{ex1Q103} puncta from SRCP1 knockouts expressing either RFP or ^{RFP}SRCP1 from (K-L) (n=4, * p<0.05). Error bars indicate SD. (N-O) Deletion of SRCP1 results in the accumulation of insoluble species. Lysates from wildtype and SRCP1 knockout cells were quantified by BCA protein assay and subjected to differential centrifugation to isolate soluble and insoluble fractions. Samples were then run on SDS-PAGE and analyzed by western blot for either ubiquitin (N) or polyglutamine-expanded proteins (0).

FIGS. 3A-30 demonstrates SRCP1 reduces levels of aggregated, but not soluble ^{GFP}Htt^{ex1Q74}. (A) Expression of SRCP1 in HEK293 cells results in a decrease in ^{GFP}Htt^{ex1Q74} puncta. HEK293 cells were transfected with either ^{GFP}Htt^{ex1Q74}, or ^{GFP}Htt^{ex1Q74} and ^{RFP}SRCP1, and imaged by fluorescent microscopy. Co-expression of ^{RFP}SRCP1 decreased ^{GFP}Htt^{ex1Q74} puncta. Shown is a representative image (n=3). (B) Quantification of the number of ^{GFP}Htt^{ex1Q74} puncta in (A). Images obtained for (A) were analyzed using ImageJ to determine the number of puncta in a field of confluent HEK293 cells (n=3, ** p<0.01). Results indicate that co-expression of SRCP1 significantly decreases the number of puncta. Error bars indicate SD. (C) Quantification of the size of ^{GFP}Htt^{ex1Q74} puncta in (A). Images obtained for (A) were analyzed using ImageJ to determine the size of puncta in cells expressing ^{GFP}Htt^{ex1Q74}, or ^{GFP}Htt^{ex1Q74} and ^{RFP}SRCP1 (n=3, ** p<0.01). Results indicate that co-expression of SRCP1 significantly decreases ^{GFP}Htt^{ex1Q74} puncta size. Error bars indicate SD. (D) SRCP1 suppresses the accumulation of aggregated ^{GFP}Htt^{ex1Q74}. HEK293 cells were transfected with either ^{GFP}Htt^{ex1Q74}, or ^{GFP}Htt^{ex1Q74} and ^{RFP}SRCP1. Cells were collected 48 hours post-transfection and analyzed by western blot. Two replicates of the experiment are shown (n=6). (E) Co-expression of ^{RFP}SRCP1 decreases aggregated ^{GFP}Htt^{ex1Q74}. The amount of ^{GFP}Htt^{ex1Q74} aggregates in (D) was quantified and standardized against a loading control (actin) (n=4, * p<0.05). Error bars indicate SD. (F) Co-expression of ^{RFP}SRCP1 does not alter levels of soluble ^{GFP}Htt^{ex1Q74}. Quantification of soluble ^{GFP}Htt^{ex1Q74} in (D). The amount of soluble ^{GFP}Htt^{ex1Q74} was quantified and standardized against a loading control (actin) (n=4). Error bars indicate SD. (G) SRCP1 reduces the levels of aggregated ^{GFP}Htt^{ex1Q74} in filter trap assays. HEK293 cells expressing either ^{GFP}Htt^{ex1Q74}, or ^{GFP}Htt^{ex1Q74} and ^{RFP}SRCP1 were lysed in NETN buffer and quantified by BCA protein assay. Samples were prepared with SDS and 40 [symbol] g of protein were used for filter trap assay. A representative image is shown (n=3). (H) Quantification of ^{GFP}Htt^{ex1Q74} present in the filter trap assay (G). The amount of ^{GFP}Htt^{ex1Q74} present in (G) was quantified using ImageJ (n=3, ** p<0.01). Error bars indicate SD. (I) Soluble, but not punctate ^{GFP}Htt^{ex1Q74} is present in cells co-expressing ^{GFP}Htt^{ex1Q74} and ^{RFP}SRCP1. Representative confocal images of HEK293 expressing ^{GFP}Htt^{ex1Q74}, or ^{GFP}Htt^{ex1Q74} and ^{RFP}SRCP1 are shown. Cells expressing ^{GFP}Htt^{ex1Q74} and ^{RFP}SRCP1 have similar levels of diffuse ^{GFP}Htt^{ex1Q74}, but reduced levels of aggregated ^{GFP}Htt^{ex1Q74}. Arrowheads indicate puncta. A representative image is shown (n=3). (J) SRCP1 does not alter levels of ^{GFP}Htt^{ex1Q23}. HEK293 cells were transfected with either ^{GFP}Htt^{ex1Q23} or ^{GFP}Htt^{ex1Q74} in the presence and absence of ^{RFP}SRCP1. Cells were collected 48 hours post-transfection and analyzed by western blot. A representative image is shown (n=3). (K) Quantification of protein levels of ^{GFP}Htt^{ex1Q23} in (J). The amount of ^{GFP}Htt^{ex1Q23} was quantified and standardized against a loading control (actin) (n=3). Error bars indicate SD. (L) SRCP1 suppresses ^{GFP}Htt^{ex1Q74} in iPSC-derived neurons. iPSC derived neurons were stained with Tuj1 (white) and astrocytes with GFAP (red). Nuclei are labeled with Hoechst (blue). Arrowheads indicate aggregated ^{GFP}Htt^{ex1Q74}; asterisks indicate diffuse ^{GFP}Htt^{ex1Q74}. Representative images are shown. (M) Quantification of ^{GFP}Htt^{ex1Q74} puncta present in (L) (n=3, *<p=0.05). Error bars indicate SD. (N) SRCP1 suppresses ^{GFP}Htt^{ex1Q74} aggregation in zebrafish spinal cord neurons. Zebrafish embryos were injected with RNA for ^{GFP}Htt^{ex1Q74}, or ^{GFP}Htt^{ex1Q74} and ^{RFP}SRCP1 and imaged 24 hours later for the presence of ^{GFP}Htt^{ex1Q74} aggregates. Representative images are shown. (O) Quantification of ^{GFP}Htt^{ex1Q74} puncta present in (N). The number of ^{GFP}Htt^{ex1Q74} aggregates were blindly scored for GFP puncta (n=10, **** p<0.0001). Error bars indicate SD.

FIG. 4 demonstrates SRCP1 is not toxic in HEK293 cells SRCP1 is not toxic to HEK293 cells. HEK293 cells were transfected with either RFP or ^{RFP}SRCP1 and viability was assessed by CellTiter-Glo assay (n=3).

FIGS. 5A-5F demonstrate SRCP1 targets aggregation-prone ^{GFP}Htt^{ex1Q74} to the proteasome for degradation. (A) Autophagy inhibition does not significantly increase soluble ^{GFP}Htt^{ex1Q74} in the presence of ^{RFP}SRCP1. HEK293 cells were transfected with either ^{GFP}Htt^{ex1Q74}, or ^{GFP}Htt^{ex1Q74} and ^{RFP}SRCP1 and treated with either DMSO or 5 mM 3-MA 24 hours post-transfection. Samples were collected 24 hours later, and levels of ^{RFP}SRCP1 and ^{GFP}Htt^{ex1Q74} were determined by western blot. Shown is a representative blot (n=3). (B) Quantification of soluble levels of ^{GFP}Htt^{ex1Q74} in (A). The amount of soluble ^{GFP}Htt^{ex1Q74} present in (A) was quantified using ImageJ (n=3, ** p<0.01). Error bars indicate SD. (C) Autophagy inhibition does not increase punctate ^{GFP}Htt^{ex1Q74} in HEK293 cells co-expressing ^{GFP}Htt^{ex1Q74} and ^{RFP}SRCP1. HEK293 cells were transfected with either ^{GFP}Htt^{ex1Q74}, or ^{GFP}Htt^{ex1Q74} and ^{RFP}SRCP1 and treated with either vehicle (DMSO) or 5 mM 3-MA 24 hours post-transfection. Cells were imaged 24 hours after treatment by fluorescent microscopy. (D) Proteasome inhibition stabilizes soluble ^{GFP}Htt^{ex1Q74} in the presence of ^{RFP}SRCP1. HEK293 cells were transfected with either ^{GFP}Htt^{ex1Q74}, or ^{GFP}Htt^{ex1Q74} and ^{RFP}SRCP1 in the presence or absence of MG132. Samples were collected 18 hours later, and levels of ^{RFP}SRCP1 and ^{GFP}Htt^{ex1Q74} were determined by western blot. Shown is a representative blot (n=3). Levels of ^{GFP}Htt^{ex1Q74} increased in both the insoluble and soluble fraction in the absence of ^{RFP}SRCP1. In cells co-transfected with ^{GFP}Htt^{ex1Q74} and ^{RFP}SRCP1 levels of soluble, but not aggregated ^{GFP}Htt^{ex1Q74} increased, consistent with a chaperoning role for SRCP1. (E) Quantification of soluble levels of ^{GFP}Htt^{ex1Q74} in (D). The amount of soluble ^{GFP}Htt^{ex1Q74} present in (D) was quantified using ImageJ (n=3, ** p<0.01). Error bars indicate SD. (F) Proteasome inhibition leads to an increase in diffuse, but not punctate ^{GFP}Htt^{ex1Q74} in HEK293 cells co-expressing ^{GFP}Htt^{ex1Q74} and ^{RFP}SRCP1. HEK293 cells were transfected with either ^{GFP}Htt^{ex1Q74}, or ^{GFP}Htt^{ex1Q74} and ^{RFP}SRCP1 for 24 hours prior to the addition of 10 μM MG132 or vehicle (DMSO). Images were taken 18 hours after addition of MG132 or DMSO by fluorescent microscopy.

FIGS. 6A-6D demonstrate ^{GFP}Htt^{ex1Q74} accelerates proteasomal degradation of SRCP1. (A)^{RFP}SRCP1 turnover is accelerated by ^{GFP}Htt^{ex1Q74}. HEK293 cells were transfected with ^{RFP}SRCP1, or ^{GFP}Htt^{ex1Q74} and ^{RFP}SRCP1. Samples were analyzed by western blot. Shown is a representative blot (n=3). (B) Quantification of levels of ^{RFP}SRCP1 in the presence or absence of ^{GFP}Htt^{ex1Q74} from (A). Levels of ^{RFP}SRCP1 were quantified using ImageJ (n=3, *** p<0.001). Error bars indicate SD. (C) SRCP1 is degraded by the proteasome. HEK293 cells were transfected with ^{RFP}SRCP1, or ^{GFP}Htt^{ex1Q74} and ^{RFP}SRCP1 for 24 hours prior to the addition of 10 μM MG132 or vehicle (DMSO). Samples were collected 18 hours later, and levels of ^{RFP}SRCP1 were determined by western blot. Shown is a representative blot (n=3). (D) Levels of ^{RFP}SRCP1 in the presence and absence of ^{GFP}Htt^{ex1Q74} and MG132 from (C) were quantified using ImageJ (n=3, * p<0.05, **p<0.01). Error bars indicate SD.

FIGS. 7A-7D demonstrate SRCP1's serine-rich domain is dispensable for SRCP1 function. (A) SRCP1 contains a serine-rich N-terminal region. Schematic depicting the N-terminal serine-rich region of SRCP1. The SRCP1^{ST1} construct has all N-terminal serine and threonine residues mutated to alanine. (B) SRCP1's serine-rich region does not suppress ^{GFP}Htt^{ex1Q74} aggregation. HEK293 cells were transfected with ^{GFP}Htt^{ex1Q74}, ^{GFP}Htt^{ex1Q74} and ^{RFP}SRCP1, or ^{GFP}Htt^{ex1Q74} and ^{RFP}SRCP1^{ST1}. Samples were collected 48 hours after transfection and analyzed for levels of ^{GFP}Htt^{ex1Q74} by western blot. No difference between ^{RFP}SRCP1 and ^{RFP}SRCP1^{ST1} was detected. Shown are three replicates (n=9). (C) The amount of ^{GFP}Htt^{ex1Q74} aggregates in (B) was quantified and standardized against a loading control (actin) (n=9, * p<0.05). Error bars indicate SD. (D) Serine and threonine residues in SRCP1's serine-rich N-terminal region do not suppress ^{GFP}Htt^{ex1Q74} puncta formation. HEK293 cells were transfected with ^{GFP}Htt^{ex1Q74}, ^{GFP}Htt^{ex1Q74} and ^{RFP}SRCP1, or ^{GFP}Htt^{ex1Q74} and ^{RFP}SRCP1^{ST1}. Samples were imaged 48 hours after transfection by fluorescent microscopy.

FIGS. 8A-8P demonstrate SRCP1's pseudo-amyloid domain prevents polyQ aggregation. (A) SRCP1's C-terminal region contains two predicted to form amyloid. Schematic depicting the sequence of SRCP1's C-terminal region. Multiple in silico programs predict an aggregation-prone, amyloid-forming region in SRCP1. Amino acids that are predicted to form amyloid are indicated by asterisks. (B) Sequence of two predicted amyloid-forming regions that are mutated in (D-F) or used as peptides (O, P). (C) SRCP1's pseudo-amyloid domain closely aligns with other glycine-rich, amyloid-forming domains. PROMALS3D multiple sequence alignment tool was used to align the amyloid-forming regions of SRCP1, α-synuclein, prion protein, and amyloid beta. (D) Amino acids 61-70 are essential for suppressing ^{GFP}Htt^{ex1Q74} aggregation. HEK293 cells were transfected with ^{GFP}Htt^{ex1Q74}, ^{GFP}Htt^{ex1Q74} and ^{RFP}SRCP1, ^{GFP}Htt^{ex1Q74} and ^{RFP}SRCP1^{61-70A}, or ^{GFP}Htt^{ex1Q74} and ^{RFP}SRCP1^{71-80A}. Cells were imaged 48 hours post-transfection by fluorescent microscopy. (E) Amino acids 61-70 of SRCP1 are essential for suppressing polyQ aggregation. HEK293 cells were transfected with ^{GFP}Htt^{ex1Q74}, ^{GFP}Htt^{ex1Q74} and ^{RFP}SRCP1, ^{GFP}Htt^{ex1Q74} and ^{RFP}SRCP1^{61-70A}, or ^{GFP}Htt^{ex1Q74} and ^{RFP}SRCP1^{71-80A}. Samples were collected 48 hours post-transfection and analyzed by western blot (n=7). (F) Levels of aggregated ^{GFP}Htt^{ex1Q74} from (E) were quantified using ImageJ (n=4, * p<0.05, ** p<0.01, *** p<0.001). Error bars indicate SD. (G) Amino acids V65 and 169 are essential for suppressing ^{GFP}Htt^{ex1Q74} aggregation. HEK293 cells were transfected with ^{GFP}Htt^{ex1Q74}, ^{GFP}Htt^{ex1Q74} and ^{RFP}SRCP1, ^{GFP}Htt^{ex1Q74} and ^{RFP}SRCP1^{V65A}, or ^{GFP}Htt^{ex1Q74} and ^{RFP}SRCP1^{I69A}. Cells were imaged 48 hours post-transfection by fluorescent microscopy. (H) A peptide derived from SRCP1's pseudo-amyloid domain suppresses polyQ aggregation. In vitro Htt^{Q46} aggregation assays were performed in the absence or presence of increasing ratios of SRCP1 peptide to Htt^{Q46}. A representative image is shown (n=5). (I) SRCP1 decreases Htt^{Q46} fibrils. In vitro Htt^{Q46} aggregation assays were performed with Htt^{Q46} and SRCP1 61-80 peptide (3:1 peptide to Htt^{Q46}) for 5 hours and imaged by EM. (J) SRCP1 decreases aggregated Htt^{Q46}. In vitro Htt^{Q46} aggregation assays were performed with Htt^{Q46} and SRCP1 61-80 peptide (3:1 peptide to Htt^{Q46}) for 5 hours. Samples were then prepared with SDS, subjected to filter trap assay, and analyzed via western blot for polyglutamine. (K) SRCP1 decreases larger Htt^{Q46} species. In vitro Htt^{Q46} aggregation assays were performed with Htt^{Q46} and SRCP1 61-80 peptide (3:1 peptide to Htt^{Q46}) for 5 hours. Samples were analyzed by dynamic light scattering. (L) SRCP1 peptide delays but does not prevent Htt^{Q46} amyloid fiber formation. In vitro Htt^{Q46} aggregation assays were performed with Htt^{Q46} and SRCP1 61-80 peptide (3:1 peptide to Htt^{Q46}) for 72 hours and imaged by EM. (M) SRCP1 peptide does not prevent Htt^{Q46} aggregation over 72 hours. In vitro Htt^{Q46} aggregation assays were performed with Htt^{Q46} and SRCP1 61-80 peptide for 72 hours (3:1 peptide to Htt^{Q46}). Samples were then prepared with SDS, subjected to filter trap assay, and analyzed via western blot for polyglutamine. (N) A SRCP1 peptide delays but does not prevent the formation of larger Htt^{Q46} species. In vitro Htt^{Q46} aggregation assays were performed with Htt^{Q46} and SRCP1⁶¹⁻⁸⁰ peptide for 72 hours (3:1 peptide to Htt^{Q46}). Samples were analyzed by dynamic light scattering. (0) A peptide of SRCP1's amino acids 61-70 suppresses polyQ aggregation. In vitro Htt^{Q46} aggregation assays were performed with Htt^{Q46} and either SRCP1 61-80 peptide, SRCP1 61-70 peptide, or SRCP1 71-80 peptide (3:1 peptide to Htt^{Q46}). Htt^{Q46} alone was used as a positive control. A representative image is shown (n=3). (P) Amino acids V65 and 169 are essential for SRCP1 61-70 peptide to suppress polyQ aggregation. In vitro Htt^{Q46} aggregation assays were performed with Htt^{Q46} and either SRCP1 61-70 peptide or SRCP1 61-70 peptide with amino acids V65 and 169 mutated to alanine (VI-A) (3:1 peptide to Htt^{Q46}). Htt^{Q46} alone was used as a positive control. A representative image is shown (n=3).

FIGS. 9A-9C demonstrate SRCP1 rescues defects in neurite outgrowth in HD iPSC-derived neurons, (A) SRCP1 increases neurite outgrowth in HD iPSC-derived neurons. HD iPSC-derived neurons were transfected with ^{RFP}SRCP1 or the transfection reagent alone (control). HD iPSC-derived neurons were stained with Tuj1 (green). Nuclei labeled with Hoechst. Representative images depict neurite length variability within the two treatment conditions. (B) Quantification of (A) shows a significant increase in neurite length (μm) in HD iPSC-derived neurons treated with ^{RFP}SRCP1 compared to the transfection reagent alone (control) (n=3, ****<p=0.0001). Error bars indicate SD. (C) Working model of SRCP1 function. (i.) Soluble polyQ-expanded proteins are expressed and are not recognized by SRCP1. (ii.) Some polyQ-expanded proteins undergo a conversion to a misfolded aggregation-prone confirmation. (iii.) In the absence of SRCP1 aggregation-prone polyQ proteins forms amyloid fibers. (iv.) SRCP1 binds amyloid-forming polyQ proteins. (v.) SRCP1 targets aggregation-prone polyQ proteins to the proteasome. (vi.) Both aggregation-prone polyQ protein and SRCP1 are degraded by the proteasome.

FIG. 10 shows a model of repeat expansion mutation wherein repeated trinucleotides add a string of glutamines (Gln) to the protein.

FIG. 11 shows the location of the repeat expansion mutation giving rise to Huntington's disease.

FIGS. 12A-12C describes SRCP1 reduces the levels of protein aggregates for other neurodegenerative disease proteins. A) Overexpression of SRCP1 reduces the levels of aggregated mutant SOD1 in HEK293 cells. HEK293 cells were transfected with either wild-type or mutant (G85R or A4V) SOD1 either in the presence or absence of SRCP1 for 48 hours. Cells were then collected and lysed prior to ultracentrifugation to isolate aggregated proteins. After ultracentrifugation cell protein in the pellet was suspended in Laemmli buffer and analyzed by SDS-PAGE and western blot B) SRCP1 results in a reduction of α-synuclein. HEK 293 cells were transfected with either wild-type or mutant α-synuclein in the presence or absence of SRCP1. Cells were collected 48 hours later and analyzed by SDS-PAGE and western blot.

### DETAILED DESCRIPTION OF THE INVENTION

The present disclosure provides, compositions, methods and kits for inhibiting protein aggregation, specifically in one embodiment, polyglutamine aggregation. Further, compositions, methods and kits for treating or preventing polyglutamine diseases, including Huntington's disease are provided. Additionally, compositions, methods and kits for treating or preventing diseases associated with protein aggregation, more preferably neurodegenerative diseases associated with protein aggregation, are provided. The compositions are able to reduce the amount of aggregated proteins within a host cell.

Unless defined otherwise, all terms used herein have the same meaning as commonly understood by one of ordinary skill in the art to which the present invention belongs. Although a certain method and a material is described herein, it should not be construed as being limited thereto, any similar or equivalent method and material to those may also be used in the practice or testing of the present invention. All publications mentioned herein are incorporated herein by reference in their entirety to disclose and describe the methods and/or materials in connection with which the publications are cited. It must be noted that as used herein and in the appended claims, the singular forms “a,” “an,” and “the” include plural referents unless the context clearly dictates otherwise.

The terms “peptide,” and “polypeptide” are used interchangeably herein to refers to a chain-type polymer formed by amino acid residues which are linked to each other via peptide bonds between the alpha-amino and carboxy groups of adjacent residues. The term “polypeptide” includes a peptide and a protein. The proteins or polypeptides may include modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “peptide” are used interchangeably herein when referring to an encoded gene product and fragments thereof that make up the inhibitor of protein aggregation. Thus, exemplary peptides or proteins include gene products, homologs, orthologs, paralogs, fragments and other equivalents, variants, and analogs of the foregoing.

Protein aggregation is a biological phenomenon in which impaired homeostatic mechanisms lead to the accumulation of proteins in the cell. This can lead to cell death and other pathologies. The phenomenon is associated with more than 71 diseases including Huntington's disease, a progressive brain disorder characterized by uncontrolled movements, emotional problems, and loss of thinking ability (cognition). Additionally, other neurodegenerative diseases are associated with protein aggregation, including, but not limited to, for example, Parkinson's disease (PD), amyotrophic lateral sclerosis (ALS), among others.

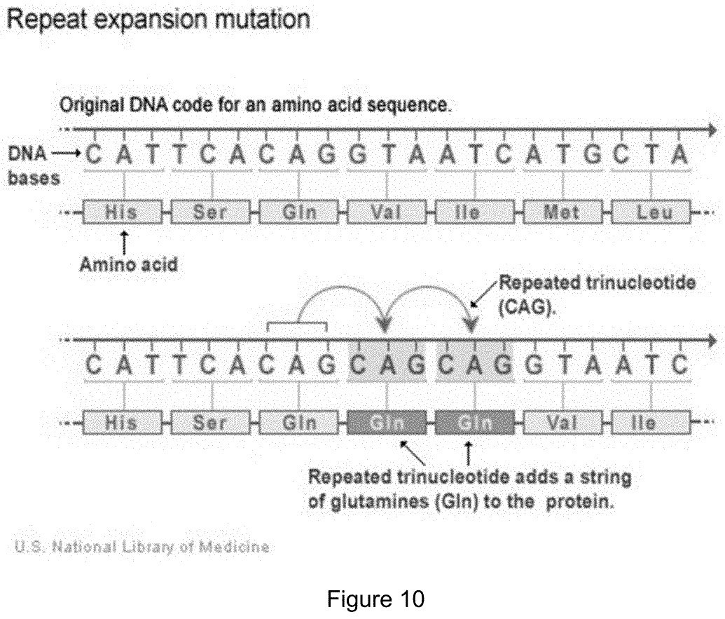

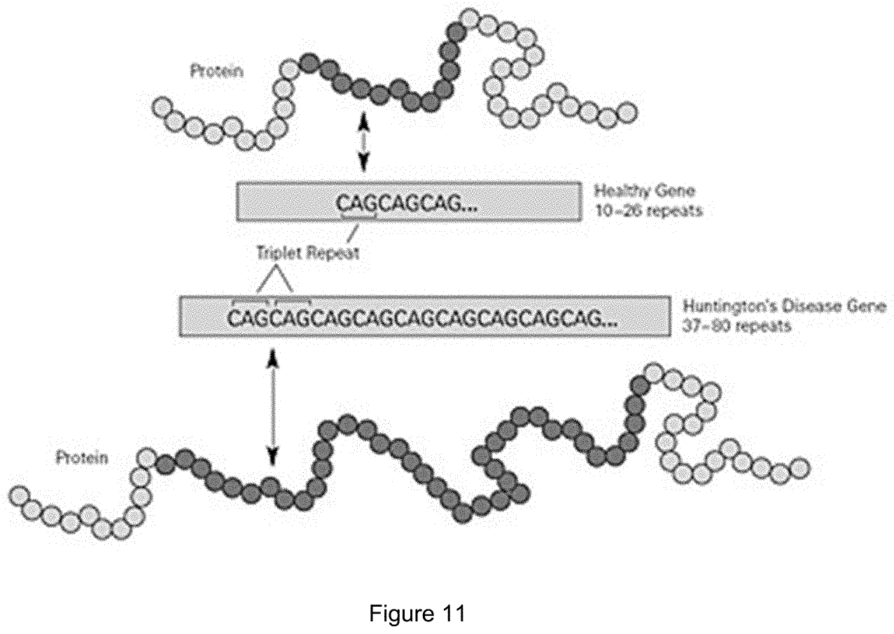

People with Huntington's have a mutation in the gene that codes for the protein called Huntingtin. The mutation is a CAG trinucleotide repeat (demonstrated in FIG. 10), a series of three nucleotides (cytosine, adenine, and guanine), that appears more than 120 times in the gene. When this gene is translated (FIG. 11), the resulting protein has longer tracts (strings) of glutamines. These polyglutamine tracts are sticky, so the proteins are prone to aggregation and accumulate in nuclei of neurons, disrupting the normal functions of these cells. The dysfunction and eventual death of neurons in certain areas of the brain are responsible for the signs and symptoms of Huntington's disease.

Amyotrophic lateral sclerosis (ALS) is a progressive neurodegenerative disease (also included as a motor neuron disease) that involves the neurons responsible for controlling voluntary muscle movement. In ALS, both the upper motor neurons and the lower motor neurons degenerate or die, and stop sending messages to the muscles. Unable to function, the muscles gradually weaken, start to twitch (called fasciculations), and waste away (atrophy). At some point, the brain loses its ability to initiate and control voluntary movements. In North America, ALS is commonly referred to as Lou Gehrig's disease, named after the famous New York Yankees baseball player who died of the disease in 1941. Familial ALS has been linked to genetic mutations in the SOD1 gene (Rosen et al. 1993). ALS results from impaired mutant copper-zinc superoxide dismutase-1 (cu-zn superoxide dismutase, now called commonly SOD1) maturation. The mutations alter the abundance of the SOD1 enzyme within cells. Over 100 different mutations in SOD1 have been linked to familiar, inherited ALS. A hallmark of ALS is the abnormal accumulation of protein aggregates (or deposits) containing the mutated SOD1. All the mutations disrupt the structure of SOD1, which, not to be bound by any theory, are thought to lead to the disrupted SOD1 becoming sticky toward itself and other protein leading to the aggregation that is present in ALS.

PD is a progressive nervous system disorder that affects movement. α-Synuclein is encoded by the SNCA gene and is a presynaptic neuronal protein found mainly at the tips of neurons in presynaptic terminals. α-Synuclein has been linked genetically and neuropathologically to Parkinson's disease (PD). α-Synuclein is thought to contribute to PD pathogenesis in a number of ways, but generally it is thought that aberrant soluble oligomeric conformations of α-Synuclein (called protofibrils), are the toxic species that mediate disruption of cellular homeostasis and neuronal death by having an effect on various intracellular targets, including synaptic function. Further, secreted α-synuclein is thought to affect neighboring cells by seeding of aggregation, thus possibly contributing to disease propagation. Further α-Synuclein is also dysregulated in other neurodegenerative conditions, termed synucleinopathies.

Here, the inventors have identified a novel Dictyostelium discoideum specific chaperone, serine rich chaperone protein 1 (SRCP1), that recognizes aggregation prone, polyglutamine expanded proteins and targets them for proteasomal degradation. In addition to suppressing polyglutamine expression in Dictyostelium, expression of SRCP1 in human cell, iPSC derived neuronal, and zebrafish models of Huntington's disease results in a suppression of huntingtin aggregation. The Examples further describe the use of the SRCP1 protein for the suppression of protein aggregation in a number of other neurodegenerative diseases, including, but not limited to, for example, Parkinson's disease, ALS, among others. This disclosure provides a new class of chaperone, SRCP1, and describe a novel route to suppressing protein aggregation, including polyglutamine aggregation.

The present disclosure demonstrates a protein from Dictyostelium, SRCP1 (SEQ ID NO:2), peptides, fragments and modified proteins thereof, is able to suppress, inhibit and reduce protein aggregation, including polyglutamine aggregation, in not only Dictyostelium but also in non-endogenous cell types, for example mammalian cells. The present disclosure provides methods of treating diseases associated with protein aggregation, including polyglutamine disease, in a subject in need thereof by administering the SRCP1 protein, peptides or modified SRCP1 proteins (via compositions, vectors, viruses and the like) which inhibit, reduce or prevent the aggregation of proteins, including polyglutamine proteins within the subject to treat or prevent at least one symptom of the disease. The ability of the peptides and SRCP1 protein to targets protein aggregates within exogenous cells, and not non-aggregated forms of the target protein, provides a benefit when treating diseases in which the non-aggregated protein plays a biologically necessary role in maintaining the subject's health. The present disclosure provides methods and compositions for reducing, inhibiting or suppressing protein aggregation, including polyglutamine aggregation, in cells and also for treating a polyglutamine disease, such as Huntington's disease.

Suitable target proteins are proteins that aggregate within the host cell leading and in some cases lead to deleterious effects, including, for example, cell death. Suitable target proteins include proteins that have multiple polyglutamine stretches and are prone to polyglutamine aggregation within a host cell. Suitable target proteins include proteins associated with polyglutamine diseases, including the proteins listed in Table 1.

TABLE 1

| Polyglutamine | | | |
|---|---|---|---|
| Disorders | Gene | Locus | Protein |
| Spinal and bulbar | AR | Xq13-q21 | Androgen |
| muscular atrophy | | | receptor |
| Huntington disease | IT15 | 4p16.3 | Huntingtin |
| Dentatorubral- | DRPLA | 12p13.31 | Atrophin 1 |
| pallidoluysian | | | |
| atrophy | | | |
| Spinocerebellar | SCA1 | 6p23 | Ataxin 1 |
| ataxia 1 | | | |
| Spinocerebellar | SCA2 | 12q24.1 | Ataxin 2 |
| ataxia 2 | | | |
| Spinocerebellar | SCA3/MJD | 14q32.1 | Ataxin 3 |
| ataxia 3 | | | |
| (Machado-Joseph | | | |
| disease) | | | |
| Spinocerebellar | CACNA1A | 19p13 | (α_{1A}-voltage- |
| ataxia 6 | | | dependent calcium |
| | | | channel subunit |
| Spinocerebellar | SCA7 | 3p12-p13 | Ataxin 7 |
| ataxia 7 | | | |
| Spinocerebellar | TBP | 6q27 | TATA box |
| ataxia 17 | | | binding protein |

In another embodiment, the suitable target proteins are proteins in which their aggregation is associated with a disease, for example, a neurodegenerative disease. For example, suitable target proteins associated with a neurodegenerative disease are provided in Table 2.

TABLE 2

| Additional | | | |
|---|---|---|---|
| Disorders | | | |
| associated with | | | |
| protein aggregation | Gene | Locus | Protein |
| Parkinson's disease | SOD-1 | | SOD-1 (copper-zinc |
| | | | superoxide dismutase) |
| ALS | SNCA | | α-Synuclein |

The present disclosure has identified amino acids 61-70 of SEQ ID NO:2 of the SRCP1 plays a role in SRCP1's ability to suppress protein aggregation, including polyglutamine aggregation, as mutation of this region results in a loss of functionality of the SRCP1 for suppressing aggregation. As such, in one embodiment the disclosure provides an isolated polypeptide comprising SEQ ID NO:5 (LIWGVYGFIR, corresponding to amino acids 61-70 of SEQ ID NO:2) as an inhibitor of protein aggregation.

Suitable other isolated peptides can be derived from SEQ ID NO:2 which contain SEQ ID NO:5 and retain the inhibitory effect of inhibiting protein aggregation. These isolated polypeptides are contemplated as part of the present invention (including, for example, but not limited to, SEQ ID NO:9, SEQ ID NO:10, SEQ ID NO:4, SEQ ID NO:6). Additional full or partial SRP1 polypeptide sequences derived from SEQ ID NO:2 are contemplated that have one or more mutations within the sequence of amino acids 61-70 are also contemplated.

The present invention in some embodiments provides isolated peptides and modified SRCP1 proteins which contain non-endogenous amino acid mutations within the wildtype (SEQ ID NO:2) protein which allow for the isolated peptide or modified SRCP1 protein to retain the ability to suppress protein aggregation, including polyglutamine aggregation. The isolated peptides or modified SRCP1 proteins contains at least amino acids 60-71 of SEQ ID NO:2 (e.g. SEQ ID NO:5) and at least one mutation within the amino acid sequence of the wildtype SRCP1 protein (SEQ ID NO:2). In some embodiments, the isolated peptide or SRCP1 protein is a peptide of SEQ ID NO:5.

All isolated peptides and modified SRCP1 proteins contemplated for use in the present invention are proteins in which the functionality of the SRCP1 protein is maintained, i.e. the peptides or modified SRCP1 protein maintains the ability to suppress, inhibit, or reduce protein aggregation, including polyglutamine aggregation. One skilled in the art would understand, using the methods described herein, how to test for the ability of the modified SRCP1 proteins to maintain functionality as a suppressor or inhibitor of protein aggregation, and these resultant peptides or modified proteins are contemplated as part of the invention.

In one embodiment, the present disclosure provides an isolated peptide inhibitor of protein aggregation comprising, consisting essentially of, consisting of SEQ ID NO:5 (LIWGVYGFIR). In one embodiment, the isolated peptide inhibitor of protein aggregation is a peptide comprising SEQ ID NO:9 (LIWGVYGFIRGGVGLVKWRG).

In some embodiments, the isolated peptide comprising SEQ ID NO:5 further comprises 1-78 additional amino acids selected from amino acids 1-60 and 71-88 of SEQ ID NO:2 (e.g., amino acids that flank SEQ ID NO:5, where SEQ ID NO:5 corresponds to amino acids 61-70 of SEQ ID NO:2). As described in the Examples, additional amino acid sequence of the SRCP1 protein (SEQ ID NO:2) can be added to the peptide comprising SEQ ID NO:5 while maintaining the ability of the peptide to suppress, inhibit, or reduce protein aggregation (for example, but not limited to, SEQ ID NO:9, SEQ ID NO:10, among others). It is to be noted herein that it is contemplated that the 1-78 additional amino acid added to SEQ ID NO:5 from 1-60 or 71-88 of SEQ ID NO:2 are added to SEQ ID NO:5 in the order of the sequence found in SEQ ID NO:2 (e.g., a portion or all of amino acids 1-60 may be added to the isolated peptide at the N-terminus of SEQ ID NO:5 and a portion or all of amino acids 71-88 may be added to the C-terminus of SEQ ID NO:5). One or more mutations within the added amino acid sequence can be located within this added flanking sequence (e.g., within amino acids 1-60 and 71-88).

For example, in one embodiment, the peptide comprises SEQ ID NO:5 and from 1-78 additional amino acids from amino acids 1-60 or 71-88 of SEQ ID NO:2 with at least one amino acid mutation within the additional sequence as long as the isolated peptide retains its functionality as an inhibitor of protein aggregation (e.g., isolated peptides having a length of 11-88 amino acids that retain the ability to suppress protein aggregation). In further embodiments, the isolated peptide comprises SEQ ID NO:5 (e.g., amino acids 61-70 of SEQ ID NO:2) and 1-78 additional amino acids selected from amino acids 1-60 or 71-88 of SEQ ID NO:2 comprising at least three or more amino acid mutations within the additional sequence, alternatively at least five or more amino acid mutations within the additional sequence, alternatively at least ten or more amino acid mutations within the additional sequence, alternatively at least fifteen or more amino acid mutations within the additional sequence, alternatively at least twenty or more amino acid mutations within the additional sequence, alternatively at least 30 or more amino acid mutations within the additional sequence, alternatively at least 35 or more amino acid mutations within the additional sequence, alternatively at least 40 or more amino acid mutations within the additional sequence, alternatively at least 45 or more amino acid mutations within the additional sequence, alternatively at least 50 or more amino acid mutations within the additional sequence.

In one embodiment, the isolated peptide may contain additional added sequences form 1-60 or 71-88 of SEQ ID NO:2 to flank SEQ ID NO:5 (e.g., a portion or all of amino acids 1-60 may be added to the isolated peptide at the N-terminus of SEQ ID NO:5, and a portion or all of amino acids 71-88 may be added to the C-terminus of SEQ ID NO:5) wherein the additional sequence may contain at least 2-50 mutations, alternatively at least 2-20 mutations, alternatively at least 2-16 mutations from the sequence of SEQ ID NO:2 (as long as the mutations do not fall within amino acids 60-71 of SEQ ID NO:2). In some embodiments, the isolated peptide contains the full length sequence of SEQ ID NO:2 or a fragment thereof (e.g., a peptide of 10-88 amino acids) in which the one or more mutations is found. One skilled in the art would be able to determine peptides of 10-88 amino acids that at least comprise SEQ ID NO:5 that retain the ability to inhibit or suppress protein aggregation.

In suitable embodiments, the at least one mutation is a substitution of one amino acid to another amino acid (e.g., an amino acid selected from alanine (A), arginine (R), asparagine (N), aspartic acid (D), cysteine (C), glutamine, (Q), glycine (G), histidine (H), isoleucine (I), leucine (L), lysine (K), methionine (M), phenylalanine (F), proline (P), serine (S), threonine (T), tryptophan (W), tyrosine (Y) or valine (V)). In one exemplary embodiment, the mutation or substitution is at least one mutation of an amino acid to an amino acid selected from glycine, alanine, valine, leucine, or isoleucine. In another embodiment, the mutation or substitution is alanine or glycine.

In one embodiment, the isolated peptides comprise SEQ ID NO:5 and do not contain any mutations within SEQ ID NO:5.

In one embodiment, the isolated peptide comprises at least one substitution of a threonine or a serine to an alanine within amino acids 1-38 of SEQ ID NO:2 or amino acids 71-88 of SEQ ID NO:2. In another embodiment, the isolated peptide comprises at least two or more substitutions of a threonine or a serine to alanine within amino acids 1-38 of SEQ ID NO:2 or amino acids 71-88 of SEQ ID NO:2.

In some embodiments, the isolated peptide comprises from 1 to 16 of the threonine or serine substituted with an alanine in amino acids 1-38 of SEQ ID NO:2. In another embodiment, all threonine or serine within amino acids 1-38 of SEQ ID NO:2 are substituted with alanine. A suitable isolated peptide contemplated comprises SEQ ID NO:6.

In another embodiment, the isolated peptide comprises substitution of all of the amino acids of 71-80 of SEQ ID NO:2 with alanine. Suitable the peptide may be SEQ ID NO: 4. In another embodiment, the peptide may be SEQ ID NO:11 (LIWGVYGFIRAAAAAAAAAA).

In some embodiments, the isolated peptide of the present invention has at least 40% sequence similarity with SEQ ID NO:2 and wherein amino acids 61-70 are SEQ ID NO:5. In another embodiment, the isolated peptide has at least 60% sequence similarity with SEQ ID NO:2 and wherein amino acids 61-70 are SEQ ID NO:5. In another embodiment, the peptide has at least 80% sequence similarity with SEQ ID NO:2 and wherein amino acids 61-70 are SEQ ID NO:5. In yet another embodiment, the peptide has at least 90% sequence similarity with SEQ ID NO:2 and wherein amino acids 61-70 are SEQ ID NO:5. In yet another embodiment, the peptide has at least 95% sequence similarity with SEQ ID NO:2 and wherein amino acids 61-70 are SEQ ID NO:5. In these embodiments, there is 100% sequence similarity in amino acids 61-70 of SEQ ID NO:2.

In another suitable embodiment, the present disclosure provides an isolated peptide comprising SEQ ID NO: 10. The cysteines found within SEQ ID NO:10 form a cysteine bond providing a cyclic peptide. Other suitable peptides with similar design are contemplated.

In another suitable embodiment, the peptides of SEQ ID NO:5 or SEQ ID NO:9 is make cyclic by adding on cysteines (C) on either end of sequence. In some embodiments, one or more extra amino acids is added to the end of the sequence before the addition of the cysteines (e.g., but not limited to, one or more amino acids (e.g., A, G, AA, GG, etc. before the C). One skilled in the art would be able to make cyclic peptides using the peptides disclosed herein which are able to maintain their ability to inhibit protein aggregation.

In another suitable embodiment, the peptide of the present invention may include repetitive peptides, e.g., peptides that contain 2 or more of the same sequences. In one exemplary embodiment, the isolated peptide may contain 2 peptide sequences in tandem, alternatively 3 peptide sequences, alternatively 4 peptide sequences, alternatively 5 peptide sequences, etc. For example, but not limited to, the isolated peptide may comprise two SEQ ID NO:5 in tandem, three SEQ ID NO:5 in tandem, four SEQ ID NO:5 in tandem, five SEQ ID NO:5 in tandem, two SEQ ID NO:9 in tandem, three SEQ ID NO:9 in tandem, four SEQ ID NO:9 in tandem, five SEQ ID NO:9 in tandem, and may include none or one or more amino acids in between the sequences as linkers of the tandem repeats (e.g., 1, 2, 3, 4, etc. amino acids between).

In one embodiment, the disclosure provides a peptide or modified SRCP1 protein of SEQ ID NO:2 that comprises at least amino acids 60-71 of SEQ ID NO:2 (e.g. SEQ ID NO:5) and at least one mutation within the amino acid sequence of the wildtype SRCP1 protein (SEQ ID NO:2). In some embodiments, the modified protein comprises SEQ ID NO:2 with at least 3 amino acids mutated, alternatively at least 4 amino acid mutated, alternatively at least about 5 amino acids mutated, alternatively at least 6 amino acids mutated, alternatively at least 7 amino acids mutated, alternatively at least 8 amino acids mutated, alternatively at least 9 amino acids mutated, alternatively at least 1-20 amino acids are mutated from the wild-type sequence, but wherein the modified protein maintains its functionality as an inhibitor of polyglutamine aggregation. As described herein, one skilled in the art would be able to test for the functionality of the inhibitor of protein aggregation in vitro and in vivo as shown in the examples.

In one embodiment, it is envisioned that one or more amino acids may be added to the end of the peptide sequence without changing its function as a inhibitor of protein aggregation. For example, one or more amino acids may be added to the N-terminus, C-terminus, or both N- and C-terminus of the isolated peptide without altering its function (e.g., 1, 2, 3, 4, 5, 6, 7, 8, 9, 10, etc. amino acids are added to SEQ ID NO:5 or SEQ ID NO:9). Suitable embodiments are able to be determined and tested in view of this specification. For example, in one embodiment, one amino acid is added to the N-terminus or C-terminus SEQ ID NO:5, alternatively one amino acid is added to the N-terminus and one amino acid is added to the C-terminus of SEQ ID NO:5, two amino acid is added to the N-terminus SEQ ID NO:5, two amino acids are added to the C-terminus of SEQ ID NO:5, alternatively two amino acid is added to the N-terminus and two amino acid is added to the C-terminus of SEQ ID NO:5, etc. In another nonlimiting example, one amino acid is added to the N-terminus or C-terminus SEQ ID NO:9, alternatively one amino acid is added to the N-terminus and one amino acid is added to the C-terminus of SEQ ID NO:9, two amino acid is added to the N-terminus SEQ ID NO:9, two amino acids are added to the C-terminus of SEQ ID NO:9, alternatively two amino acid is added to the N-terminus and two amino acid is added to the C-terminus of SEQ ID NO:9, etc.

In one embodiment, the modified protein is a peptide comprising SEQ ID NO:5. In one embodiment, the SRCP1 protein is SEQ ID NO:5. In some embodiments, the peptide further includes a tag.

As used herein, the term “mutated” or “mutation” refers to both substitutions of amino acids or a deletion of an amino acid within the wildtype sequence to produce a modified sequence. In a preferred embodiment, the mutation is a substitution. In a preferred embodiment, the substitution is of a threonine or serine to an alanine. In another embodiment, any amino acid may be substituted to an alanine. Other amino acid substitutions are also contemplated within the scope of the invention.

In one embodiment, the peptide or modified SRCP1 protein may contain at least one amino acid mutation in SEQ ID NO: 2 within amino acids 1-60 or 71-88 of SEQ ID: 2, alternatively may contain at least 2-50 mutations, alternatively at least 2-20 mutations, alternatively at least 2-16 mutations.

In one embodiment, the modified protein comprises SEQ ID NO:2 with at least one mutation of a threonine or a serine within amino acids 1-38 of SEQ ID NO: 2 to alanine. In another embodiment, the modified protein comprises SEQ ID NO:2 with at least two or more substitutions of a threonine or a serine within amino acids 1-38 of SEQ ID NO: 2 to alanine. In a further embodiment, the modified proteins comprising SEQ ID NO:2 comprising from 1 to 16 of the threonine or serine substituted with an alanine in amino acids 1-38 of SEQ ID NO: 2. Alternatively, 5-16 of the threonine and serine are substituted with alanine in amino acids 1-38 of SEQ ID NO:2. In another embodiment, the modified protein comprises SEQ ID NO:2 wherein every serine and threonine within amino acids 1-39 are substituted with alanine, but the other amino acids in SEQ ID NO: 2 are from the wild-type protein.

In some embodiments, the modified SRCP1 proteins have substantial identity to the wildtype SRCP1 protein found in SEQ ID NO:2. In some embodiments, the modified proteins has at least 50% identity to SEQ ID NO:2, alternatively at least 75% sequence identity, alternatively at least 80% sequence identity, alternatively at least 90% sequence identity, alternatively at least 95% sequence identity, alternatively at least 99% sequence identity.

It is contemplated in some of these embodiments, there is at least one mutation within the modified SRCP1 protein from the wild-type sequence. In some preferred embodiments, the modified protein has at least 100% sequence identity within amino acids 60-71 of SEQ ID NO:2.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

In a preferred embodiment, substantial identity of amino acid sequences for purposes of this invention means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%.

In one embodiment, the modified protein is SEQ ID NO: 6. In another embodiment, the modified protein is SEQ ID NO:4. In another embodiment, the modified protein is SEQ ID NO:5.

In some embodiments, the isolated peptide, modified protein or recombinant form of the protein further contains an exogenous tag or agent.

The term “tag” or “agent” as used herein includes any useful moiety that allows for the purification, identification, detection, diagnosing, imaging, or therapeutic use of the proteins and peptides of the present invention. The terms tag or agent includes epitope tags, detection markers and/or imaging moieties, including, for example, enzymatic markers, fluorescence markers, radioactive markers, among others. Additionally, the term tag or agent includes therapeutic agents, small molecules, and drugs, among others. The term tag or agent also includes diagnostic agents.

In one embodiment, the tag is a peptide tag (e.g., but not limited to, 6HIS (HHHHHH), cMyc (EQKLISEEDL) or FLAG (DYKDDDDK), HA-tag (YPYDVPDYA), NE-tag (TKENPRSNQEESYDDNES), Xpress tag (DLYDDDD) among others). In another embodiment, the tag is a purification tag.

In some embodiments, the SRCP1 protein or peptide or modified protein is directly or indirectly linked to an exogenous tag or agent. The suitable tag or agent does not interfere with the functionality of the SRCP1 protein, peptide or modified proteins' function in reducing or inhibiting protein aggregation.

In other embodiments, the tag or agent is a polypeptide, wherein the polypeptide is translated concurrently with the peptide or SRCP1 polypeptide sequence.

Suitable tags are known in the art and include, but are not limited to, affinity or epitope tags (nonlimiting examples include, e.g., cMyc (EQKLISEEDL), HIS (e.g. 6HIS (HHHHHH), FLAG (DYKDDDDK), V5-tag (GKPIPNPLLGLDST), HA-tag (YPYDVPDYA), NE-tag (TKENPRSNQEESYDDNES), S-tag (KETAAAKFERQHMDS), Ty tag (EVHTNQDPLD)), florescence tags (RFP, GFP, etc.), polyglutamate tag (EEEEEE). Epitope tags are commonly used as a purification tag. A purification tag is an agent that allows isolation of the polypeptide from other non-specific proteins. Suitable agents further include agents that help with the bioavailability or targeting of the protein or peptide. In some embodiments, the peptide or modified SRCP1 protein is encoded in a nucleic acid sequence that encodes both peptide or modified SRCP1 protein and the tag (for example a peptide tag, or epitope tag including, but not limited to, a FLAG, HIS or HA tag).

In one embodiment of the invention, the polypeptide of the invention is linked with an agent, for example, with a detectable marker, preferably a fluorescent, enzymatic or a luminescent marker. Examples of suitable enzymes include, but are not limited to, horseradish peroxidase, alkaline phosphatase, beta-galactosidase, glucose-6-phosphatase, or acetylcholinesterase. Examples of suitable tags comprising prosthetic group complexes include, but are not limited to, streptavidin/biotin and avidin/biotin. Examples of suitable fluorescent materials include, but are not limited to, fluorescein, fluorescein isothiocyanate, rhodamine, dichlorot[pi]azinylamine fluorescein, green fluorescent protein (GFP), red fluorescent protein (RFP), blue fluorescent dyes excited at wavelengths in the ultraviolet (UV) part of the spectrum (e.g. AMCA (7-amino-4-methylcoumarin-3-acetic acid); Alexa Fluor 350), green fluorescent dyes excited by blue light (eg. FITC, Cy2, Alexa Fluor 488), red fluorescent dyes excited by green light (e.g. rhodamines, Texas Red, Cy3, Alexa Fluor dyes 546, 564 and 594), or dyes excited with infrared light (e.g. Cy5) to be visualized with electronic detectors (CCD cameras, photomultipliers); dansyl chloride, phycoerythrin or the like.

As used herein, the term “conjugate” refers to the joining of two entities by covalent bonds. The entities may be covalently bonded directly or through linking groups using standard synthetic coupling procedures. For examples, two polypeptides may be linked together by simultaneous polypeptide expression typically referred to as a fusion or chimeric protein. One or more amino acids may be inserted into polypeptide as a linking group by incorporation of corresponding nucleic acid sequences into the expression vector. Other contemplated linking groups include polyethylene glycols or hydrocarbons terminally substituted with amino or carboxylic acid groups to allow for amide coupling with polypeptides having amino acids side chains with carboxylic acid or amino groups respectively. Alternatively the amino and carboxylic acid groups can be substituted with other binding partners such as an azide and an alkyne which undergo copper catalyzed formation of triazoles.

In another example of conjugation, polypeptides are expressed to contain naturally or non-naturally occurring amino acids containing a thiol group. The thiol group can be substituted for an amino group in coupling reactions with carboxylic acids, or two thiol groups when exposed to oxidative conditions react to form disulfides. Additionally, in some embodiments, non-naturally occurring amino acids are incorporated into the polypeptide, allowing for site-specific conjugation of the polypeptide to one or more agents. For example, in one embodiment, the use of selenocysteine allows for the site-specific conjugation of the polypeptides of the present invention to suitable agents.

In some embodiments, the disclosure provides a vector comprising (a) a nucleic acid sequence encoding the isolated peptide described herein and (b) a heterologous nucleic acid sequence. In one embodiment, the peptide or protein selected from the group consisting of: (i) SRCP1 protein of SEQ ID NO: 2, (ii) a fragment of the SRCP1 protein comprising at least amino acids 61-70 of SEQ ID NO: 2; (iii) a peptide of SEQ ID NO:5; (iv) a modified protein of SRCP1 described herein, (v) an isolated peptide described herein and (b) a heterologous nucleic acid sequence, wherein the vector expresses the peptide, protein, fragment or modified protein in a host cell. In some embodiments, the protein is (iv), and the nucleic acid encodes for the protein selected from the group consisting of SEQ ID NO:6 (STA mutation of N terminus) and SEQ ID NO:4 (SRCP1 71-80 mutant). In another embodiment, the vector comprises a nucleic acid sequence encoding the peptide of SEQ ID NO:5.

The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments to the targeted protein. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of exogenous genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors, such as viral vectors (e.g., replication defective retroviruses, adenoviruses and adena-associated viruses), which serve equivalent functions.

In some embodiments, the heterologous sequence of the vector is a viral sequence. Suitable viral sequences include, but are not limited to, an adeno-associated viral sequence or a retroviral sequence. In a preferred embodiment, the heterologous nucleic acid sequence is a recombinant adeno-associated virus.

In some embodiments, the virus is an adeno-associated virus (rAAV), a lentivirus, an adnovirus, a herpes simplex virus, a baculovirus, pox virus, alphaviruses, among others. Further embodiments include viruses made using the viral vectors described herein. Suitable methods for making the viruses are known in the art.

In one embodiment, the virus is an AAV-vector or rAAV vector. AAV vectors have been shown in the art as useful delivery vehicles for gene therapy due to lack of toxicity and demonstrating long-term transgene expression (McCarty et al., Ann. Rev. Genet. 38:819-845 (2004)). As used herein, the term “rAAV vector” refers to an AAV vector carrying a nucleic acid sequence encoding a functional gene (i.e a polynucleotide of interest that expresses the peptide described herein). The rAAV vectors contain 5′ and 3′ adeno-associated virus inverted terminal repeats (ITRs), and the polynucleotide of interest operatively linked to sequences, which regulate its expression in a target cells, within the context of the invention. In one embodiment, the rAAV vector encompasses individual rAAV vector systems and rAAV-based dual vector systems. In one embodiment, the rAAV vector belongs to a AAV serotype selected in a group comprising AAV1, AAV2, AAV3, AAV4, AAV5, AAV8, AAV9, AAV10, and rhesus macaque-dehved serotypes including AAVrhlO, and mixtures thereof (i.e. a rAAV hybrid vector). As used herein, the term “rAAV hybrid vector”, herein designates a vector particle comprising a native AAV capsid including an rAAV vector genome and AAV Rep proteins, wherein Cap, Rep and the ITRs of the vector genome come from at least 2 different AAV serotypes. The hybrid vector of the invention may be for instance a rAAV2/4 vector, comprising an AAV4 capsid and a rAAV genome with AAV2 ITRs or a rAAV2/5 vector, comprising an AAV5 capsid and a rAAV genome with AAV2 ITRs. In one embodiment, said rAAV is AAV2/2, AAV2/4 serotype or AAV2/5 serotype.

In some embodiments, the peptides, protein or modified proteins are comprised within a heterologous gene construct. “Genetic construct” can include nucleic acid sequences that permit it to replicate in the host cell. Examples include, but are not limited to a vector, plasmid, cosmid, bacteriophage, or virus that carries exogenous DNA into a cell. A genetic construct can also include additional selectable marker genes and other genetic elements known in the art.

A vector can preferably transduce, transform or infect a cell, thereby causing the cell to express the nucleic acids and/or proteins encoded by the vector.

Further embodiments provide a composition comprising the isolated peptides described herein, the SRCP1 protein of SEQ ID NO:2; a modified SRCP1 protein described herein; a peptide comprising SEQ ID NO:5, a vector comprising the peptides or SRCP1 proteins described herein, fragments thereof or modified proteins thereof; or a virus able to express the isolated peptide, SRCP1 protein or modified SRCP1 protein described herein. The compositions may further comprise a pharmaceutically acceptable carrier.

A “pharmaceutically acceptable carrier” means any conventional pharmaceutically acceptable carrier, vehicle, or excipient that is used in the art for production and administration of compositions to a subject. Pharmaceutically acceptable carriers are typically non-toxic, inert, solid or liquid carriers which are physiologically balanced. Pharmaceutically acceptable carriers are well known to those skilled in the art and include, but are not limited to, 0.01 to 0.1 M and including 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. Typically phosphate buffer saline or other saline solutions are physiologically acceptable carriers. Water is not contemplated as a suitable physiologically acceptable carrier. In some embodiments, additional components may be add to preserve the structure and function of the viruses, vectors or proteins of the present invention, but are physiologically acceptable for administration to a subject. Protein stabilizers may be added to maintain the structure of the protein and reduce degradation. In one embodiment, the peptide is solubilized in DMSO and then diluted in buffer.

The pharmaceutical composition may be in unit dosage form. In such form the preparation is divided into unit doses containing appropriate quantities of the active component (e.g., isolated peptide). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilizing and/or dispersing agents. Alternatively the active ingredient may be in powder form for constitution with a suitable vehicle, e.g. sterile, pyrogen-free water, before use.

The pharmaceutically acceptable carrier may further comprise a preservative. The term “preservative” as used herein refers to a chemical compound which is added to a pharmaceutical composition to prevent or delay microbial activity (growth and metabolism) of aty least one organism (e.g., bacteria, yeast, etc.) within the composition. Pharmaceutically acceptable preservatives are known in the art and can include, but are not limited to, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. The use of a preservative in pharmaceutical compositions is well-known to the skilled person. For convenience reference is made to Remington: The Science and Practice of Pharmacy, 19th edition, 1995.

In some embodiments, the composition according to the present invention may comprise an isotonic agent such as mannitol, sorbitol, glycerol, propylene glycol or a mixture thereof. In one embodiment the isotonicity agent is not a salt. The use of an isotonic agent in pharmaceutical compositions is well-known to the skilled person. For convenience reference is made to Remington: The Science and Practice of Pharmacy, 19th edition, 1995.

The pharmaceutical composition according to the invention may also comprise a buffer. The buffer may be selected from a buffer which is a zwitterionic buffer, glycyl-glycine, TRIS, bicine, HEPES, MOBS, MOPS, TES and mixtures thereof. Further suitable buffers are sodium acetate, sodium carbonate, citrate, histidine, glycine, lysine, arginine, sodium dihydrogen phosphate, disodium hydrogen phosphate, sodium phosphate, and tris(hydroxymethyl)-aminomethan, tricine, malic acid, succinate, maleic acid, fumaric acid, tartaric acid, aspartic acid or mixtures thereof.

Compositions of the present disclosure may include liquids or lyophilized or otherwise dried formulations and may include diluents of various buffer content (e.g., Tris-HCl, acetate, phosphate), pH and ionic strength, additives such as albumin or gelatin to prevent absorption to surfaces, detergents (e. g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The peptides described herein may be formulated into a shelf-stable pharmaceutical composition. The term “shelf-stable pharmaceutical composition” means a pharmaceutical composition which is stable for at least the period which is required by regulatory agencies in connection with therapeutic proteins. Preferably, a shelf-stable pharmaceutical composition is stable for at least one year at 5° C. Stability includes chemical stability as well as physical stability.

The term “stabilizer” as used herein refers to chemicals added to peptide containing pharmaceutical compositions in order to stabilize the peptide, i.e. to increase the shelf life and/or stability of such compositions. Examples of stabilizers used in pharmaceutical formulations are L-glycine, L-histidine, arginine, polyethylene glycol, and carboxymethylcellulose.

So as to improve resistance to degradation, it may be necessary to use a protected form of the peptide according to the invention. The form of protection must obviously be a biologically compatible form and must be compatible with a use in the field of pharmacy.

Many forms of biologically compatible protection may be contemplated. They are well known to the person skilled in the art as, for example, the acylation or acetylation of the amino terminal end, or the amidation or esterification of the carboxy terminal end. Thus, the invention relates to a composition such as previously defined, characterized by the fact that the peptide of SEQ ID No. 2, 4-6, 9-10 is in protected or unprotected form. Protection based on a substitution on the amino terminal end by an acetyl group, a benzoyl group, a tosyl group or a benzyloxycarbonyle group may be utilized. Preferably, protection based on the amidation of the hydroxyl function of the carboxy terminal end by an NYY group with Y representing an alkyl chain from C1 to C4, or the esterification by an alkyl group is utilized. It is also possible to protect the two ends of the peptide.

In the domain of amino acids, the molecules have a geometry such that they may theoretically be present in the form of different optical isomers. Thus, there exists a molecular conformation of the amino acid (AA) that rotates the plane of polarized light to the right (dextrorotatory conformation or D-aa), and a molecular conformation of amino acid (aa) that rotates the plane of polarized light to the left (levorotatory conformation or L-aa). Natural amino acids are always of levorotatory conformation; consequently, a peptide of natural origin will only be constituted of L-aa type amino acids. However, chemical synthesis in laboratory enables amino acids with the two possible conformations to be prepared. From this base material, it is possible to incorporate, during peptide synthesis, amino acids in both dextrorotatory and levorotatory optical isomer forms. Thus, amino acids constituting the peptide according to the invention may be in L- and D-configurations; preferentially, the amino acids are in L form. The peptide according to the invention may thus be in L-, D- or DL-form.

The peptide described herein may be obtained either by conventional chemical synthesis (in solid phase or in homogeneous liquid phase), or by enzymatic synthesis (Kullman et al., J. Biol. Chem. 1980, 225, 8234), from constituent amino acids or their derivatives or produced within a host cell.

The at least isolated peptide of the present technology can be formulated in to dosage forms to be administered orally. These dosage forms include but are not limited to tablet, capsule, caplet, troche, lozenge, powder, suspension, syrup, solution, oral thin film (OTF), oral strips, inhalation compounds or suppositories. Preferred oral administration forms are capsule, tablet, solutions and OTF. Solid dosage forms can optionally include the following types of excipients: antiadherents, binders, coatings, disintegrants, fillers, flavors and colors, glidants, lubricants, preservatives, sorbents and sweeteners.

The disclosure further provides methods and kits for reducing, inhibiting or suppressing protein aggregation, including polyglutamine aggregation in a host cell. The method comprises introducing within the host cell: (i) the isolated peptides described herein; (ii) the SRCP1 protein of SEQ ID NO: 2, (ii) a fragment of the SRCP1 protein comprising at least amino acids 61-70 of SEQ ID NO: 2; (iii) a modified protein described herein, (iv) a peptide of SEQ ID NO:5, (v) a vector described herein able to express the SRCP1 protein or peptides described, modified SRCP1 protein or fragments thereof, or (vi) a virus able to express the SRCP1 protein or peptides described, modified SRCP1 protein or fragments thereof, in an effective amount to reduce, inhibit or suppress protein aggregation. In a suitable embodiment, the method reduces, inhibits or suppresses polyglutamine aggregation.

In some embodiments, the host cell is a mammalian cell, for example, a mouse cell, a dog cell, a cow cell, a primate cell, a human cell. In a preferred embodiment, the cell is a primate cell, preferably a human cell.

In some embodiments, the method reduces, inhibits or suppresses protein aggregation by at least 20%, alternatively at least 40%, alternatively at least 60%, alternatively at least 80%, alternatively at least 90% within the host cell.

The disclosure also provides methods and kits for treating a polyglutamine disease in a subject in need thereof, the method comprising the steps of: (a) administering to the subject an isolated peptide, SRCP1 protein or modified SRCP1 protein, vector, virus or composition as described herein capable of introducing the peptide, SRCP1 protein, fragment thereof or SRCP1 modified protein or fragment thereof into the subject in an effective amount to reduce, inhibit or prevent at least one symptom of the polyglutamine disease. In one embodiment the polyglutamine disease is selected from the group consisting of SBMA; Huntington's disease; spinocerebellar ataxia type 1, spinocerebellar ataxia type 2, spinocerebellar ataxia type 3, spinocerebellar ataxia type 6, spinocerebellar ataxia type 7, spinocerebellar ataxia type 17; and dentatorubral-pallidoluysian atrophy (DRPLA).

In a preferred embodiment, the polyglutamine disease is Huntington's disease and the target protein is polyglutamine huntingtin protein. The methods specifically target the altered target protein, e.g. huntingtin protein, that is prone to aggregation, and does not alter the levels of non-aggregating target protein, huntingtin protein in a host cell.

In some embodiments, the target protein is a protein listed in Table 1.

The present compositions, methods and kits described herein may be used to treat other known known diseases caused by CAG repeat expansion besides Huntington's disease, including, but not limited to, for example, SBMA; Huntington's disease; spinocerebellar ataxia types 1, 2, 3, 6, 7, and 17; and dentatorubral-pallidoluysian atrophy (DRPLA). Because all nine inherited neurodegenerative disorders are associated with expanded polyglutamine tracts in the disease proteins, they are collectively known as polyglutamine diseases (Table 1). FIG. 10 depicts the polyglutamine disorders, the gene and protein of the nine polyglutamine diseases that may be treated by the compositions, methods and kits of the present disclosure.

In another embodiment, the disclosure provides a methods and kits for treating a disease associated with aggregation of a protein in a subject in need thereof, the method comprising the steps of administering to the subject an isolated peptide, SRCP1 protein or modified SRCP1 protein, vector, virus or composition as described herein capable of introducing the isolated peptide, SRCP1 protein or fragment thereof or SRCP1 modified protein or fragment thereof into the subject in need thereof in an effective amount to reduce, inhibit or prevent at least one symptom of the disease. Effective amounts will also be able to reduce, inhibit or prevent protein aggregation associated with the disease.

In one embodiment, the disease is a neurodegenerative disease associate with protein aggregation. Not to be bound by any theory, but the diseases, in some cases, are associated with mutant forms of a target protein which allows for protein aggregation of the mutant target protein (alone or with other proteins) within the subject. This protein aggregation leads to one or more symptoms of the disease.

Thus, the methods of the present disclosure reduces, inhibits or prevents protein aggregation within the subject of the target protein. Suitable methods comprise the steps of administering to the subject a SRCP1 protein or modified SRCP1 protein, vector, virus or composition as described herein capable of introducing the SRCP1 protein or fragment thereof or SRCP1 modified protein or fragment thereof into the subject in need thereof in an effective amount to reduce, inhibit or prevent protein aggregation associated with the disease.

In one embodiment, the disease is Parkinson's disease and the protein is SOD-1. In another embodiment, the disease is ALS and the protein is α-Synuclein.

In some embodiments, kits for carrying out the methods are provided. Suitable kits of the invention may include an isolated peptide, a SRCP1 protein, a modified SRCP1 protein, a SRCP1 peptide of SEQ ID NO:5, a vector comprising the SRCP1 protein, modified SRCP1 protein or peptides described here, or a virus able to express the SRCP1 protein, modified SRCP1 protein or peptides described herein. Suitably, the kit may also include a pharmaceutically acceptable carrier and instructions for the administration or use.

The term “treating” or “treatment” includes, but is not limited to, reducing, inhibiting or preventing one or more signs or symptoms associated with the disease or disorder. For example, treating Huntington's disease include, for example, reduction tremors, memory loss, lack of coordination, chorea, motor dysfunction, sleep disturbances, dementia, cognitive impairment, and the like. In another nonlimiting example, symptoms of ALS include but are not limited to, gradual onset, generally painless, progressive muscle weakness, tripping, dropping things, abnormal fatigue of the arms and/or legs, slurred speech, muscle cramps and twitches, and/or uncontrollable periods of laughing or crying. In a further non-limiting example, symptoms of Parkinson's disease include, but are not limited to, for example, tremor, muscle stiffness, difficulty with body movements, involuntary movements, daytime sleepiness, fatigue, poor balance, amnesia, dementia, impaired voice, soft speech, voice box spasms, loss of smell, trembling, neck tightness, weight loss among others.

Unlike other treatment methods, the treatment method of the present technology is able to prevent the development of certain protein aggregation associated diseases, including polyglutamine diseases, as the treatment methods are able to reduce or inhibit the formation of protein aggregation, including polyglutamine aggregation. The reduction in the aggregation in turn allows for the halting or preventing of the symptoms of the disease, as the symptoms are associated with accumulation of protein aggregations which in turn leads to cell, for example, neuronal cell death, which in turn lead to the symptoms of the protein aggregation related disease, including polyglutamine aggregation diseases.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The terms “effective amount” or “therapeutically effective amount” refer to an amount sufficient to effect beneficial or desirable biological and/or clinical results.

The present invention also provides methods of targeting protein aggregates, for example, but not limited to, polyglutamine aggregates for degradation within a host cell. The method includes introducing an isolated peptide, SRCP1 protein or modified SRCP1 protein into a host cell in an effective amount to reduce the amount of protein aggregates, for example but not limited to, polyglutamine aggregates. In some embodiments, the amount of protein aggregates is reduced by at least 10%, alternatively at least 20%, alternatively at least 30%, alternatively at least 50%, alternatively at least 70% in the host cell.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The invention will be more fully understood upon consideration of the following non-limiting examples.

### EXAMPLES

### Example 1: Identification of Novel Dictystelium discoideum Chaperone Protein that Suppressed Polyglutamine Aggregation

This examples demonstrates that Dictyostelium are naturally resistant to protein aggregation. This Examples shows that this suppression of polyglutamine aggregation is not due to Hsp70, Hsp90, autophagy, and the ubiquitin proteasome system in Dictyostelium. Using a forward genetic screen, this Example identified a single Dictyostelium discoideum specific gene that is responsible for suppressing polyglutamine aggregation. This gene encodes a small 9.1 kDa serine rich chaperone protein (SRCP1) that suppresses polyglutamine aggregation in Dictyostelium, in mammalian cells, including human cells (including neurons), and in a zebrafish model of Huntingtin's disease. SRCP1 functions by selectively recognizing aggregation prone Huntingtin protein, but not soluble Huntingtin protein, and targeting it to the proteasome for degradation. The SRCP1 protein can be used in other cell types to prevent aggregation, allowing its adaption to a therapy for treatment of polyglutamine diseases, including Huntington's disease.

In addition to targeting polyQ-expanded, aggregation-prone protein for degradation, SRCP1 also suppresses polyQ aggregation in the presence of proteasome inhibitor, consistent with a chaperone function for SRCP1. SRCP1 does not contain any identifiable chaperone domains, but rather utilizes a C-terminal pseudo-amyloid domain to suppress aggregation of polyQ-expanded proteins. Further, this example demonstrates a small 10 amino acid fragment that confers the ability to suppress aggregation.

### Results

SRCP1 is Necessary for Dictyostelium discoideum to Evade polyQ Aggregation

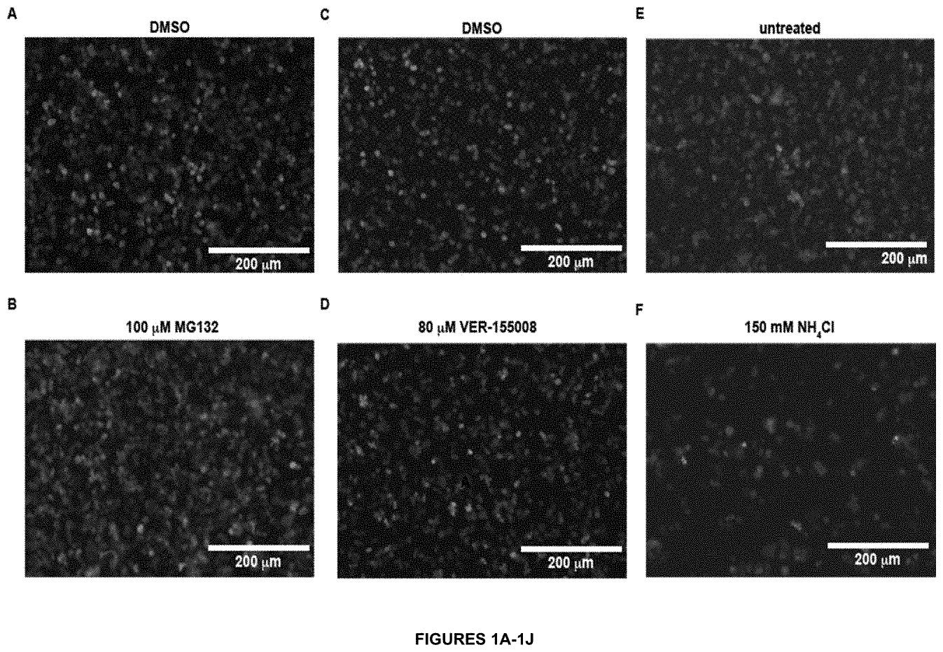

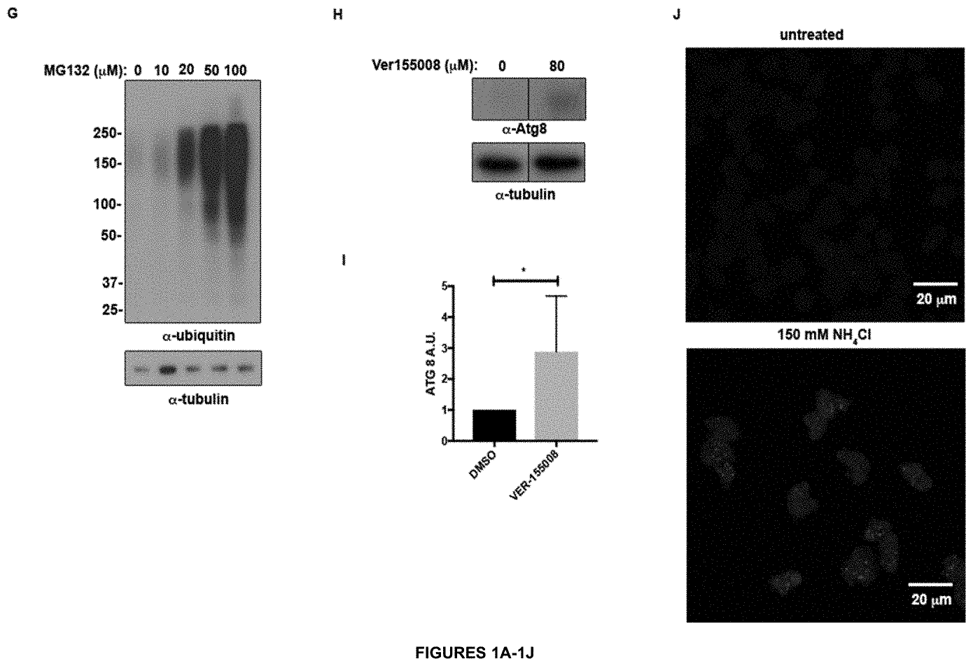

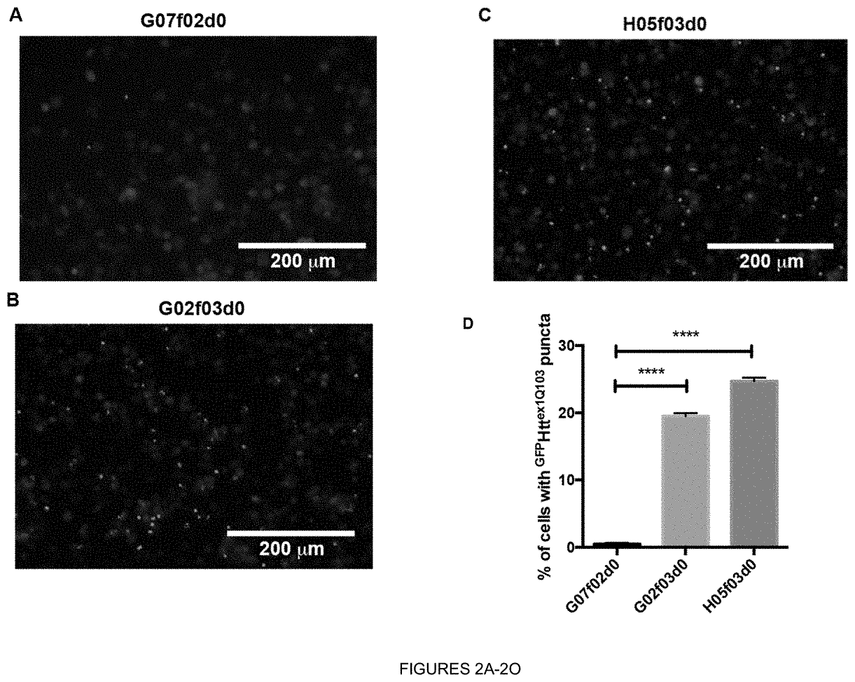

Among known protein quality control pathways, molecular chaperones, autophagy, and the ubiquitin-proteasome pathway assist in combating polyQ aggregation (Koyuncu et al., 2017; Kuiper et al., 2017; Nath and Lieberman, 2017; Sambataro and Pennuto, 2017). To determine if these pathways suppress polyQ aggregation in Dictyostelium, we stably expressed ^{GFP}Htt^{ex1Q103} in Dictyostelium and inhibited select protein quality control pathways. Inhibition of known protein quality control components, including Hsp70, autophagy, and the ubiquitin-proteasome system did not lead to an accumulation of ^{GFP}Htt^{exQ103} puncta (FIG. 1A-J), suggesting that other protein quality control pathways are responsible for Dictyostelium's unusual resistance to polyQ aggregation.

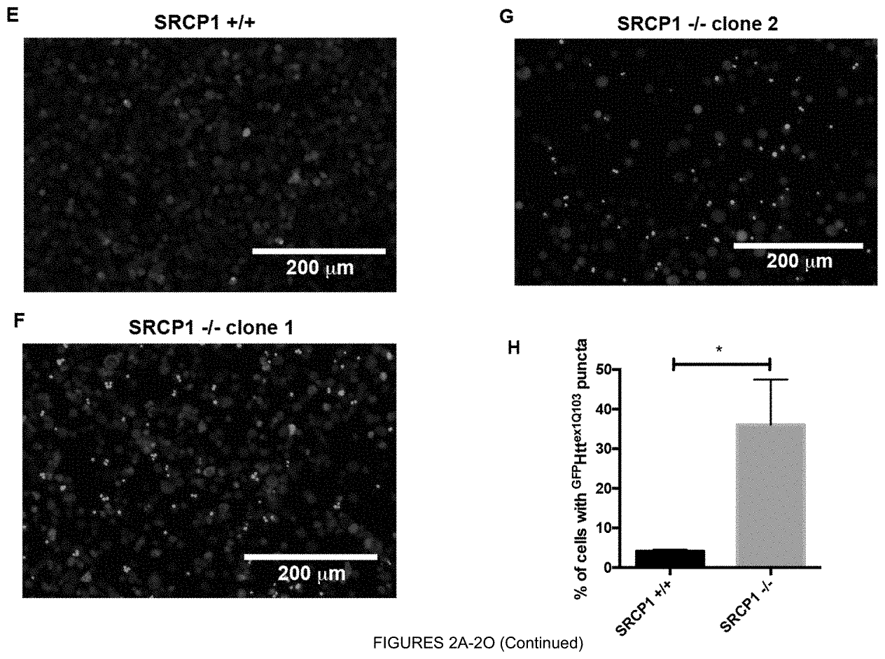

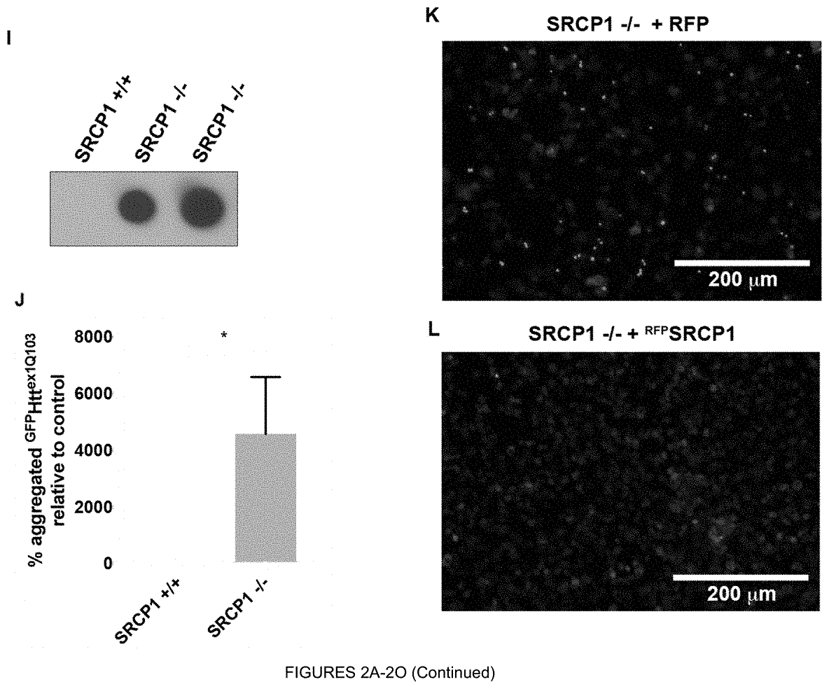

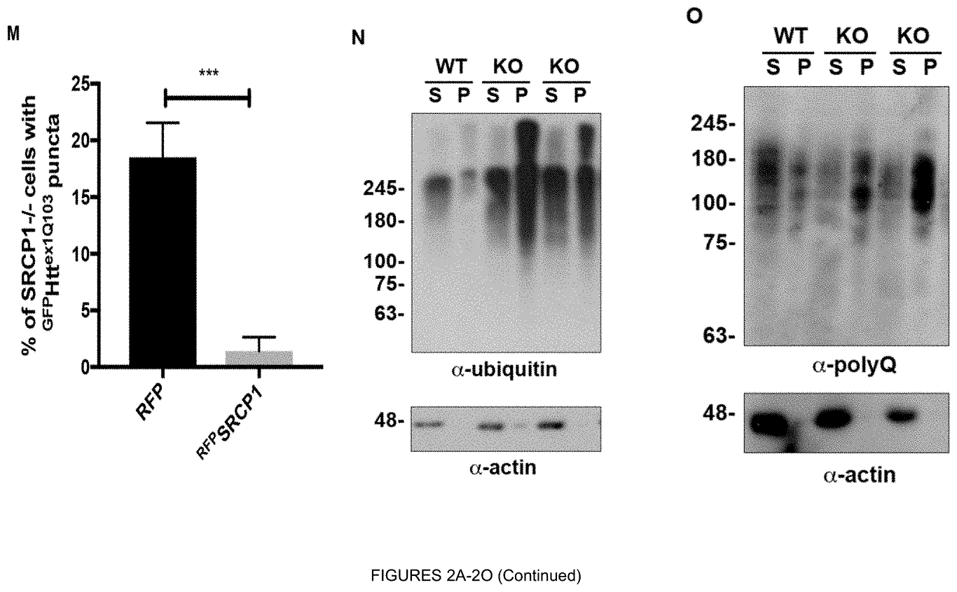

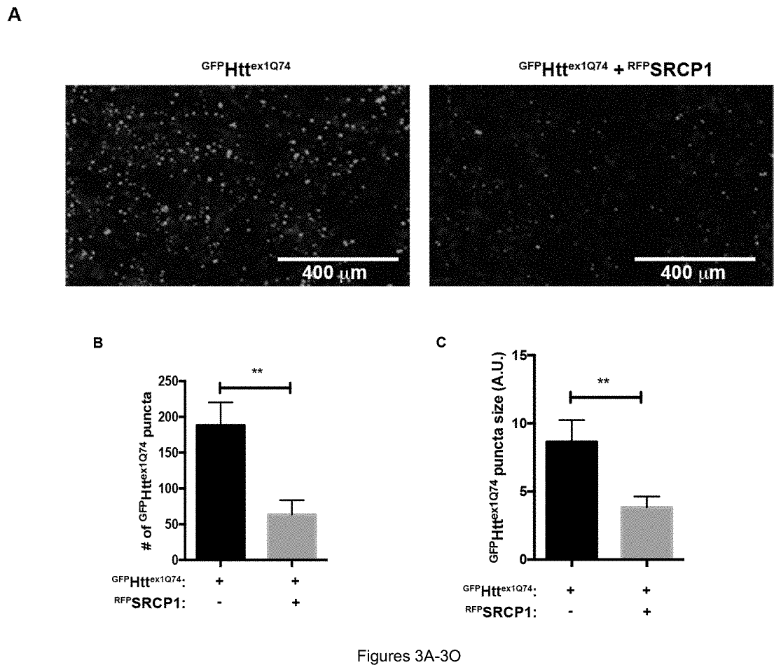

We next utilized a forward genetic screen to identify genes responsible for suppressing polyQ aggregation in Dictyostelium. We performed a restriction enzyme-mediated integration (REMI) screen in Dictyostelium stably expressing ^{GFP}Htt^{ex1Q103} and coupled it with high-content imaging to identify clonal isolates where a minimum of 5% of the cells contained ^{GFP}Htt^{ex1Q103} puncta. From this screen we identified a single uncharacterized Dictyostelium discoideum specific gene responsible for suppressing ^{GFP}Htt^{ex1Q13} aggregation (FIG. 2A-D). The protein encoded by this gene is a member of a large gene family of undefined function that encodes proteins with a serine-rich domain; therefore, we named it serine-rich chaperone protein 1 (SRCP1). To confirm that SRCP1 is responsible for suppressing ^{GFP}Htt^{ex1Q103} aggregation, we generated SRCP1 knockout Dictyostelium strains. Knocking out SRCP1 led to no obvious growth phenotypes (data not shown), while expression of ^{GFP}Htt^{ex1Q103} in SRCP1−/− cells resulted in the formation of numerous ^{GFP}Htt^{ex1Q103} puncta (FIG. 2E-H) that are insoluble via filter trap assay consistent with a role for SRCP1 in suppressing polyQ aggregation (FIG. 21, J). We next wanted to confirm that our results were due to removal of SRCP1, and not an indirect effect. To accomplish this, we transformed SRCP1−/− cells with ^{GFP}Htt^{ex1Q13} and either RFP or ^{RFP}SRCP1. Consistent with SRCP1 suppressing ^{GFP}Htt^{ex1Q103} aggregation, expression of ^{RFP}SRCP1, but not RFP alone, prevented ^{GFP}Htt^{ex1Q13} aggregation (FIG. 2K-M). Together these data are consistent with SRCP1 being responsible for Dictyostelium discoideum's unusual resistance to polyQ aggregation.

We next wanted to determine SRCP1's endogenous function in Dictyostelium. Because Dictyostelium contain an abnormal number of homopolymeric amino acid tracts, we hypothesized that SRCP1 played a role in maintaining proteostasis in Dictyostelium's repeat-rich proteome. To test this hypothesis, we collected lysates from two independent SRCP1−/− cell lines and performed differential centrifugation to separate soluble and insoluble fractions. Consistent with SRCP1 playing an important role in maintaining proteostasis in Dictyostelium, we observed an accumulation of ubiquitinated species in the insoluble fraction of the SRCP1−/− cells consistent with the presence of polyubiquitinated protein aggregates (FIG. 2N). In addition to polyubiquitinated proteins, the insoluble fraction was also enriched for endogenous polyQ proteins in the absence of SRCP1, suggesting that SRCP1 functions, at least in part, to maintain Dictyostelium's polyQ-rich proteome (FIG. 2O). Together these data suggest that SRCP1 plays an important role in maintaining proteostasis in Dictyostelium.

SRCP1 is Sufficient to Suppress polyQ Aggregation

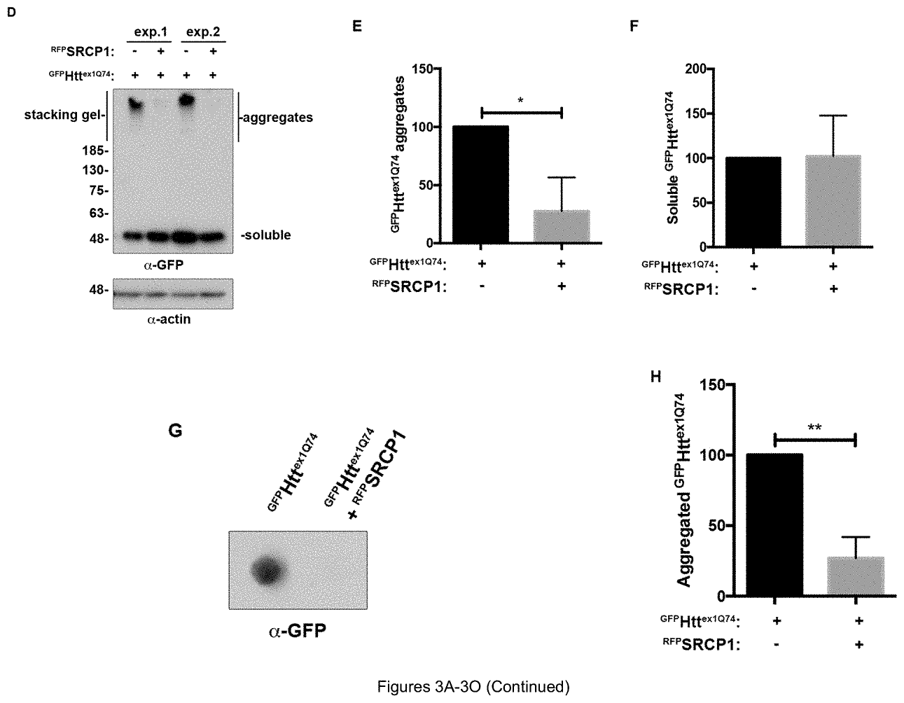

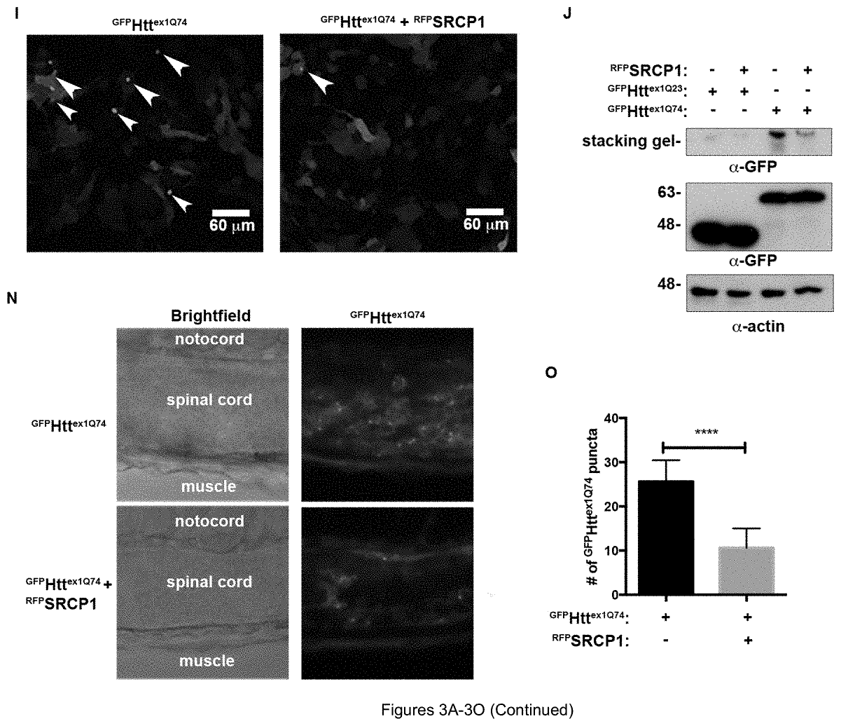

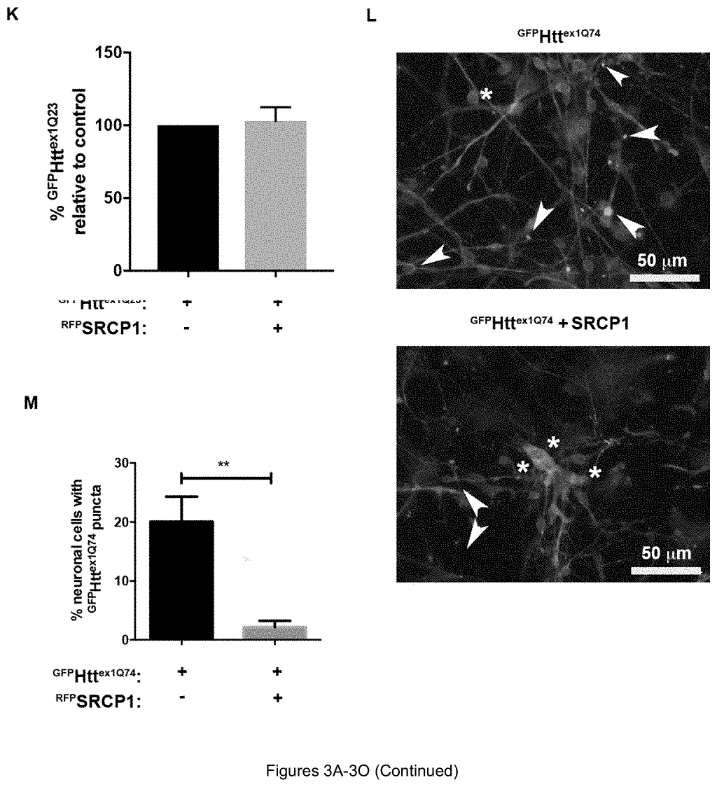

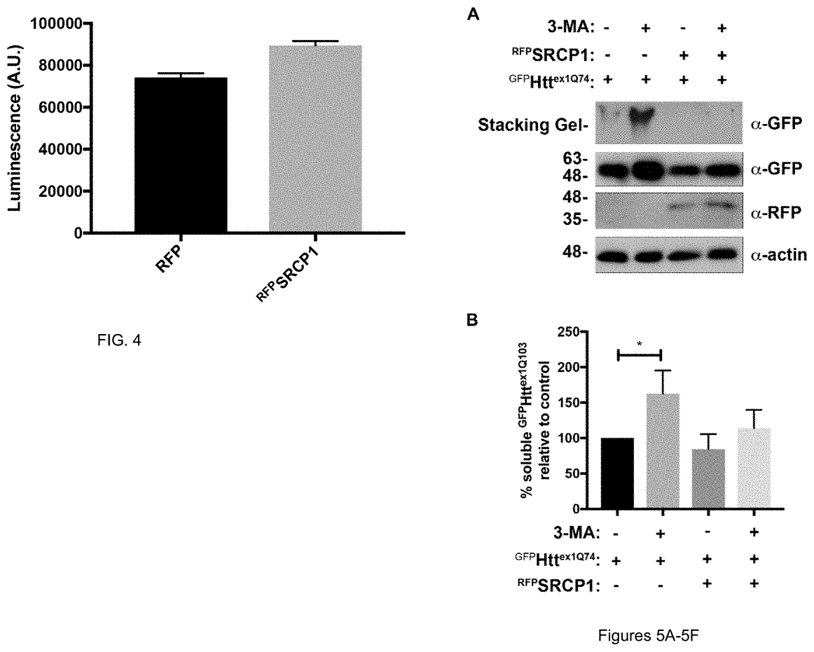

Because reducing polyQ aggregation in patients is one potential therapeutic avenue for the polyQ diseases, we next assessed the ability of SRCP1 to suppress ^{GFP}Htt^{ex1Q74} aggregation in human cells. To test this, we co-transfected HEK293 cells with ^{GFP}Htt^{ex1Q74} in the presence or absence of ^{RFP}SRCP1 and assessed the ability of ^{RFP}SRCP1 to prevent ^{GFP}Htt^{ex1Q74} aggregation. Expression of ^{GFP}Htt^{ex1Q74} resulted in the formation of ^{GFP}Htt^{ex1Q74} puncta, while co-expression of ^{RFP}SRCP1 led to a dramatic decrease in the number and size of ^{GFP}Htt^{ex1Q74} puncta (FIG. 3A-C). Importantly, expression of ^{RFP}SRCP1 alone did not display any toxicity (FIG. 4). PolyQ aggregates migrate more slowly in SDS-PAGE gels and form high molecular weight aggregates (Scherzinger et al., 1999a). To determine if SRCP1 suppressed the formation of polyQ aggregates, we next analyzed the amount of ^{GFP}Htt^{ex1Q74} aggregates in the presence or absence of ^{RFP}SRCP1. Expression of ^{GFP}Htt^{ex1Q74} resulted in the presence of both soluble ^{GFP}Htt^{ex1Q74} and ^{GFP}Htt^{ex1Q74} aggregates (FIG. 3D). Co-expression of ^{RFP}SRCP1 with ^{GFP}Htt^{ex1Q74} greatly reduced the amount of ^{GFP}Htt^{ex1Q74} aggregates as measured by SDS-PAGE, filter trap assay, and confocal microscopy, while having no effect on the levels of monomeric, soluble ^{GFP}Htt^{ex1Q74} (FIG. 3D-I). Consistent with SRCP1 having no effect on soluble polyQ protein, SRCP1 did not alter the levels of ^{GFP}Htt^{ex1Q23} suggesting that SRCP1 can selectively target polyQ-expanded proteins that have a propensity to form aggregates (aggregation-prone) (FIG. 3J, K). Because the polyQ diseases are neurodegenerative diseases, we next tested if SRCP1 prevented polyQ aggregation in human neurons. To this end, induced pluripotent stem cell (iPSC) derived neurons were transfected with either ^{GFP}Htt^{ex1Q74} alone, or ^{GFP}Htt^{ex1Q74} and ^{RFP}SRCP1. Similar to HEK293 cells, co-expression of ^{RFP}SRCP1 led to a dramatic decrease in ^{GFP}Htt^{ex1Q74} puncta (FIG. 3L, M). In addition to human neurons, we also assessed SRCP1's ability to suppress polyQ aggregation in an intact animal. We injected zebrafish embryos with RNA encoding either ^{GFP}Htt^{ex1Q74}, or ^{GFP}Htt^{ex1Q74} and ^{RFP}SRCP1, and counted the number of ^{GFP}Htt^{ex1Q74} puncta in spinal cord neurons. Similar to human cells, co-expression of ^{RFP}SRCP1 in zebrafish led to a significant reduction of ^{GFP}Htt^{ex1Q74} puncta, consistent with SRCP1 suppressing ^{GFP}Htt^{ex1Q74} aggregation (FIG. 3N, O). Together, these data demonstrate that SRCP1 is sufficient to suppress polyQ aggregation in human cells and in zebrafish neurons.

SRCP1 Prevents polyQ Aggregation and Targets Aggregation-Prone polyQ for Proteasomal Degradation

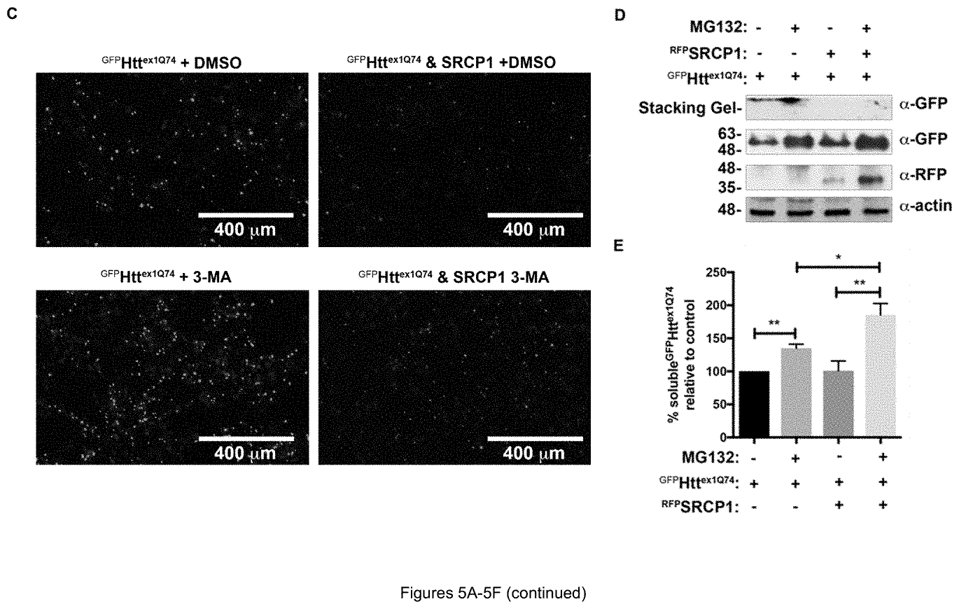

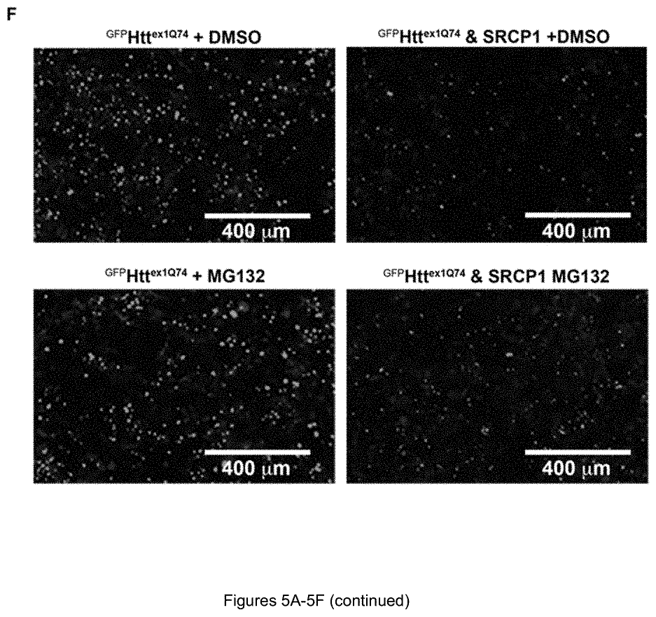

In human cells co-expression of ^{RFP}SRCP1 and ^{GFP}Htt^{ex1Q74} led to a dramatic decrease in ^{GFP}Htt^{ex1Q74} aggregates but did not cause a corresponding increase in soluble ^{GFP}Htt^{ex1Q74} levels (FIG. 3D, F). This suggests that SRCP1 was either increasing the clearance of ^{GFP}Htt^{ex1Q74} aggregates or targeting ^{GFP}Htt^{ex1Q74} that mis-folds for degradation prior to aggregation. To begin testing this, we transfected HEK293 cells with ^{GFP}Htt^{ex1Q74} in the presence or absence of ^{RFP}SRCP1 and treated with the autophagy inhibitor 3-MA 24-hours post-transfection. Consistent with previous publications, treatment with 3-MA led to a significant increase in soluble and aggregated ^{GFP}Htt^{ex1Q74} (Qin et al., 2003; Ravikumar et al., 2002). However, under conditions of autophagy inhibition, co-expression of ^{RFP}SRCP1 did not lead to a significant increase in soluble ^{GFP}Htt^{ex1Q74} levels, suggesting that SRCP1 does not target ^{GFP}Htt^{ex1Q74} for clearance via autophagy (FIG. 5A-C).

Because inhibiting autophagy did not increase levels of soluble ^{GFP}Htt^{ex1Q74} in the presence of SRCP1, we next turned our attention to the proteasome, the other major route for ^{GFP}Htt^{ex1Q74} degradation (Michalik and Van Broeckhoven, 2004). To determine if SRCP1 was stimulating proteasomal degradation of ^{GFP}Htt^{ex1Q74}, HEK293 cells were transfected with ^{GFP}Htt^{ex1Q74} in the presence or absence of ^{RFP}SRCP1 and treated with proteasome inhibitor 24 hours post-transfection. Proteasome inhibition led to increased levels of both soluble and aggregated ^{GFP}Htt^{ex1Q74} (Michalik and Van Broeckhoven, 2004; Miller et al., 2005; Waelter et al., 2001) (FIG. 5D-F). However, co-expression of ^{RFP}SRCP1 in the presence of proteasome inhibition led to an even further increase in the amount of soluble ^{GFP}Htt^{ex1Q74} when compared to proteasome inhibition alone indicating that SRCP1 targets aggregation-prone, polyQ-expanded protein for degradation via the proteasome (FIG. 5D-F). Furthermore, in the presence of proteasome inhibition, SRCP1 prevented polyQ aggregation, consistent with SRCP1 functioning as a molecular chaperone (FIG. 5D-F). Together, these data demonstrate that SRCP1 targets aggregation-prone ^{GFP}Htt^{ex1Q74} to the proteasome, but not the lysosome, for degradation, and that SRCP1 acts as a molecular chaperone preventing ^{GFP}Htt^{ex1Q74} aggregation upon conditions where ^{GFP}Htt^{ex1Q74} degradation is impaired.

PolyQ Accelerates the Co-Degradation of SRCP1

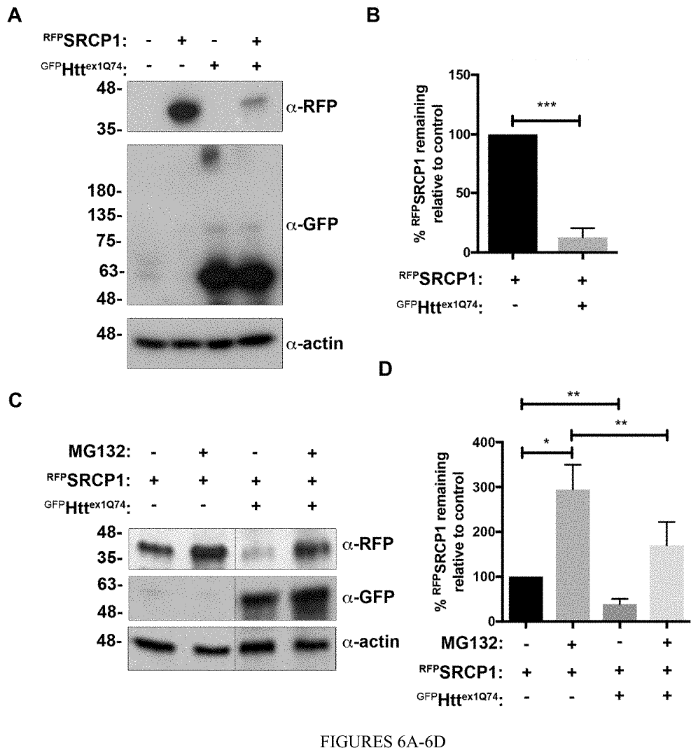

In the experiments where ^{RFP}SRCP1 and ^{GFP}Htt^{ex1Q74} were co-transfected, we observed that ^{RFP}SRCP1 levels were difficult to detect. However, in experiments where ^{RFP}SRCP1 was transfected alone, we could easily detect ^{RFP}SRCP1. This led us to hypothesize that ^{GFP}Htt^{ex1Q74} accelerated the turnover of ^{RFP}SRCP1. We next analyzed levels of ^{RFP}SRCP1 in human cells expressing ^{GFP}Htt^{ex1Q74}, ^{RFP}SRCP1, or ^{GFP}Htt^{ex1Q74} and ^{RFP}SRCP1, and found that the presence of ^{GFP}Htt^{ex1Q74} led to a dramatic decrease in ^{RFP}SRCP1 levels (FIG. 6A, B). To determine if ^{RFP}SRCP1 is degraded by the proteasome, we transfected human cells with ^{RFP}SRCP1 in the presence or absence of proteasome inhibition and assessed levels of ^{RFP}SRCP1. Proteasome inhibition led to a marked stabilization of ^{RFP}SRCP1, consistent with ^{RFP}SRCP1 being degraded by the proteasome (FIG. 6C, D). Together, these data demonstrate that ^{RFP}SRCP1 is degraded by the proteasome in a manner that is stimulated by the presence of ^{GFP}Htt^{ex1Q74}.

SRCP1's Serine-Rich Domain is Dispensable for SRCP1 Function

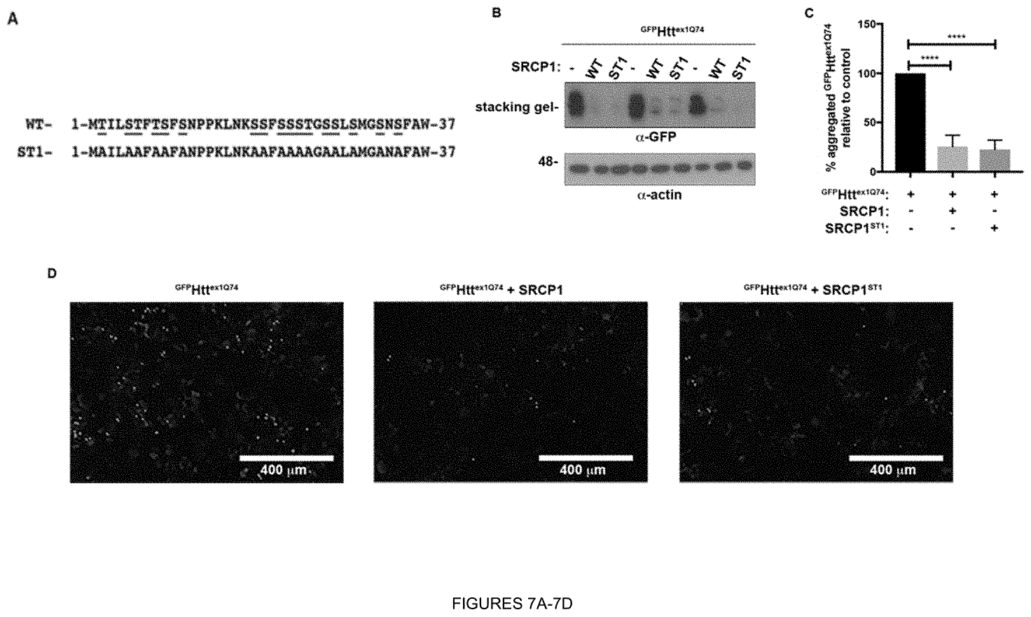

SRCP1's ability to suppress polyQ aggregation in the presence of proteasome inhibition is consistent with SRCP1 being a novel molecular chaperone. SRCP1 does not contain a canonical chaperone domain; however, it does contain a serine-rich N-terminus. Because DNAJB6 utilizes a serine-rich domain to suppress polyQ aggregation (Kakkar et al., 2016), we hypothesized that SRCP1's serine-rich N-terminus may be important for suppressing polyQ aggregation. To test this, we generated constructs where all serine and threonine residues in SRCP1's N-terminus were mutated to alanine (FIG. 7A). However, mutation of SRCP1's serine and threonine residues (^{RFP}SRCP1^{ST1}) did not disrupt SRCP1's ability to suppress polyQ aggregation (FIG. 7B-D). Together, these data demonstrate that SRCP1's serine-rich domain is dispensable for its ability to suppress polyQ aggregation.

SRCP1 Utilizes a Pseudo-Amyloid Domain to Suppress polyQ Aggregation

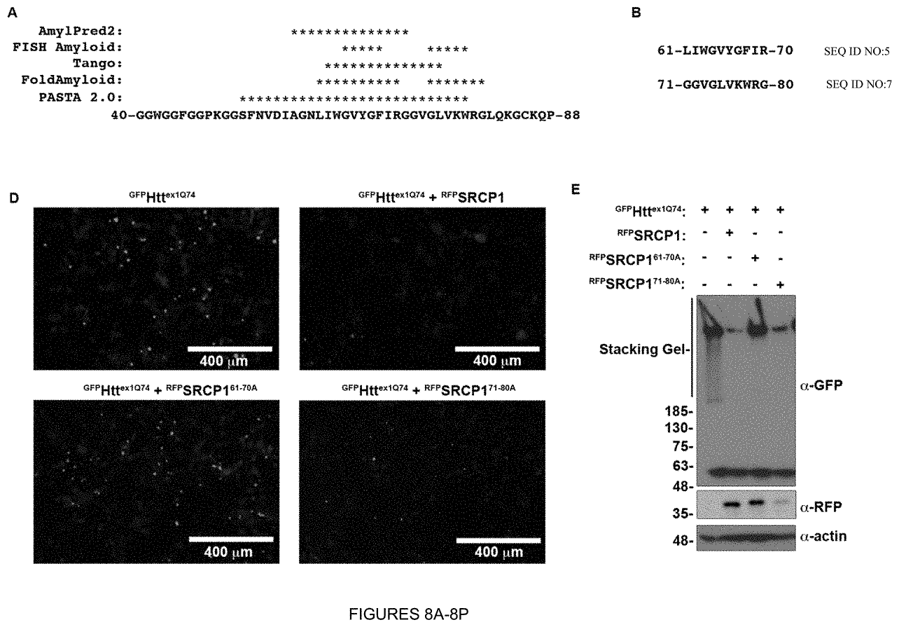

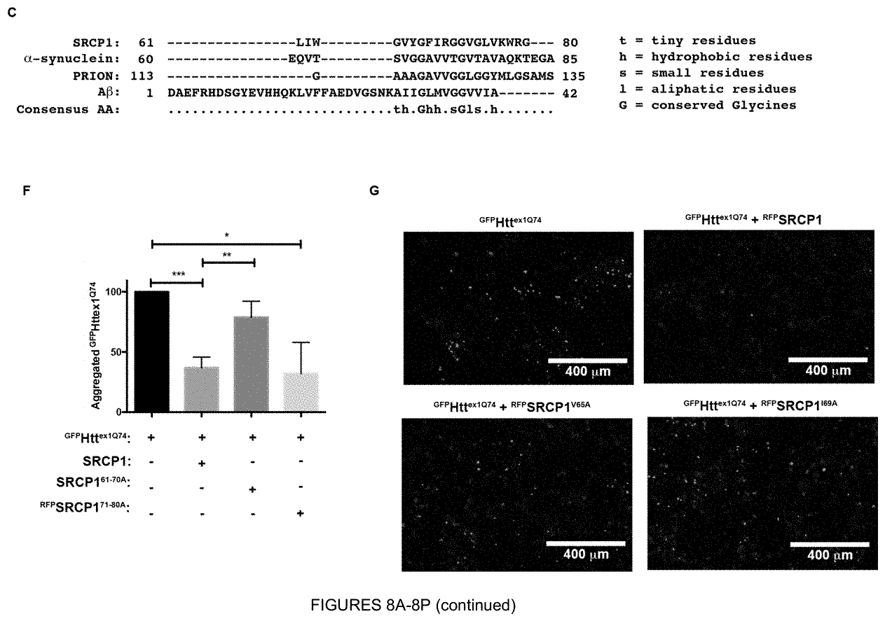

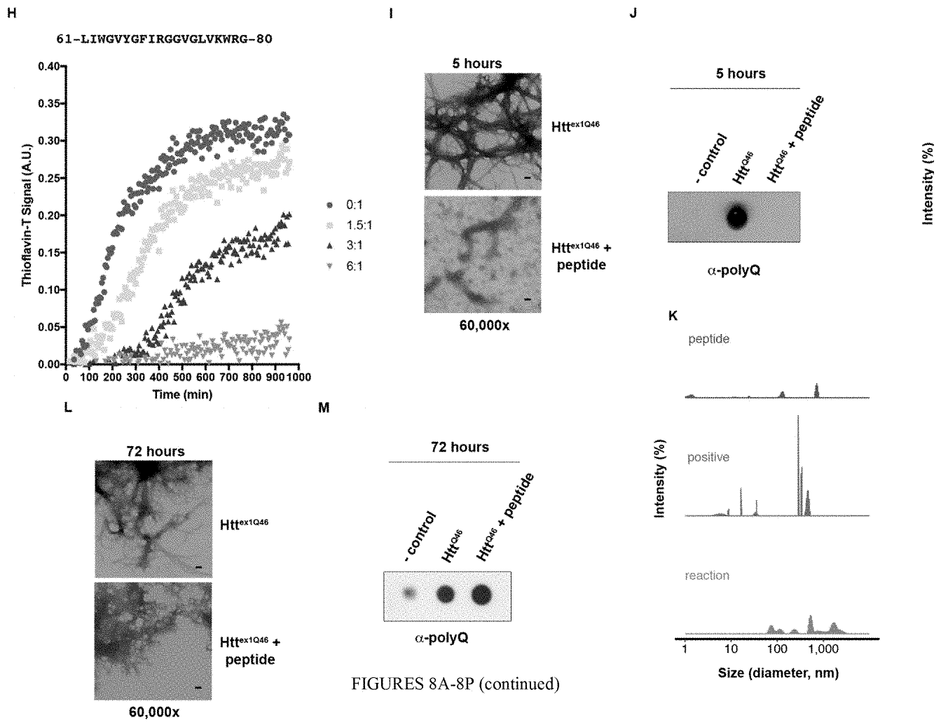

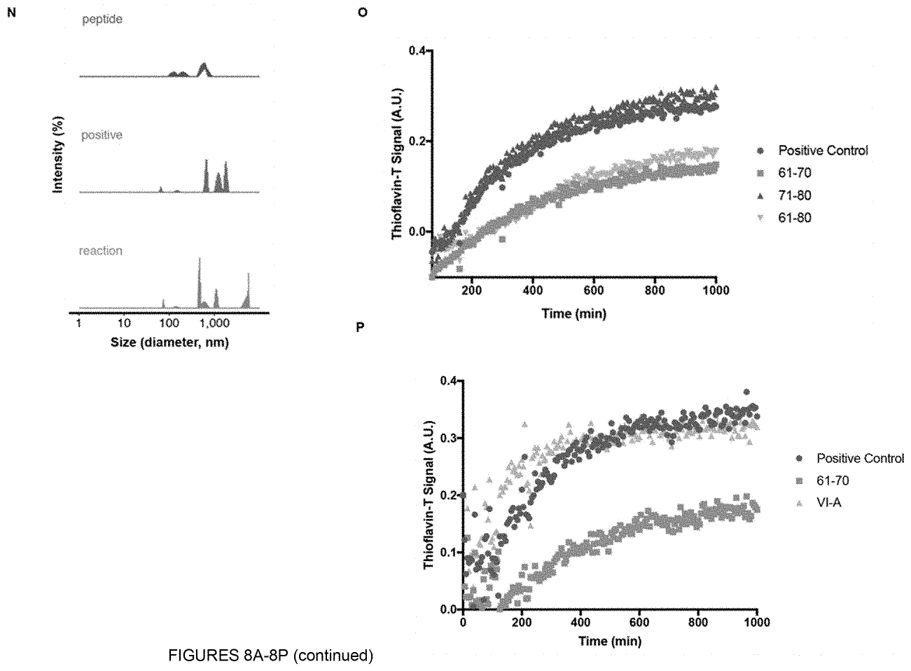

Because mutating ˜40% of the residues in SRCP1's N-terminal serine-rich domain did not cause any measurable defect in SRCP1 activity, we next turned our attention to SRCP1's C-terminal region to identify a region that may be important for SRCP1's chaperone function. One striking feature of SRCP1's C-terminal region is a highly hydrophobic, glycine-rich region that resembles amyloid. Peptides that resemble amyloid can form mixed amyloid with amyloid-forming proteins and influence amyloid formation (Cheng et al., 2012; Sato et al., 2006). This led us to hypothesize that SRCP1 utilizes a pseudo-amyloid domain to form mixed amyloid and selectively target ^{GFP}Htt^{ex1Q74} that has formed an alternate, aggregation-prone conformation. To determine if SRCP1 encodes any predicted amyloidogenic domains, we utilized in silico approaches, including Tango, FISH Amyloid, FoldAmyloid, PASTA 2.0, and AmylPred2 and identified two potential amyloid-forming regions in SRCP1's C-terminal region (FIG. 8A, B). We next aligned this C-terminal region of SRCP1 with three glycine-rich regions of amyloid-forming domains that cause neurodegenerative diseases using PROMALS3D (Pei et al., 2008) and found that SRCP1's predicted C-terminal region had high sequence homology to known amyloid-forming domains (FIG. 8C). We next mutated regions within SRCP1's predicted amyloid-forming motifs (^{RFP}SRCP1^{61-70A} and ^{RFP}SRCP1^{71-80A}) and determined their ability to suppress ^{GFP}Htt^{ex1Q74} aggregation. Consistent with SRCP1's first predicted amyloid-forming domain being important for suppressing polyQ aggregation, the ^{RFP}SRCP1^{61-70A} mutant lost the ability to suppress ^{GFP}Htt^{ex1Q74} aggregation, while the ^{RFP}SRCP1^{71-80A} mutant retained full activity (FIG. 8D-F). This is consistent with amino acids 61-70, but not 71-80 of SRCP1 containing critical residues for SRCP1 function (FIG. 8D-F). To gain more detailed insight into residues important for SRCP1 function, we next mutated individual amino acid residues in SRCP1 from 61-70 to alanine and determined their ability to suppress ^{GFP}Htt^{ex1Q74} aggregation via fluorescence microscopy. Two individual point mutations, ^{RFP}SRCP1^{V65A} and ^{RFP}SRCP1^{I69A}, resulted in a decrease in SRCP1 function, consistent with an important role for suppressing polyQ aggregation (data not shown, FIG. 8G).

We next wanted to determine if SRCP1 could directly suppress polyQ aggregation in vitro. Because we were unable to generate soluble recombinant SRCP1, we generated a 20-amino acid peptide (SEQ ID NO:9) that mimics SRCP1's predicted amyloid-forming C-terminal region. This peptide remained soluble in vitro (data not shown) and was sufficient to suppress aggregation of Htt^{Q46} in vitro as measured by Thioflavin-T fluorescence (FIG. 8H). We next wanted to confirm that our SRCP1 derived peptide was inhibiting Htt^{Q46} aggregation and not disrupting the binding of Thioflavin-T to Htt^{Q46} aggregates. To accomplish this, we analyzed Htt^{Q46} aggregate formation at a 5 hour time point by electron microscopy (EM) and found that the addition of the SRCP1 peptide decreased Htt^{Q46} fiber formation and led to the appearance of spherical structures similar to previously described soluble oligomers (Tsigelny et al., 2008) (FIG. 8I). The presence of these spherical structures and decrease in fibers also correlated with a decrease in Htt^{Q46} aggregates as measured by filter trap assay and an increase in smaller species via dynamic light scattering (DLS) (FIG. 6J, K). This, coupled with the observation that the 1.5:1 and 3:1 molar ratios of peptide to Htt^{Q46} resulted in a delay, but not prevention of Htt^{Q46} aggregation (FIG. 6H), led us to hypothesize that the SRCP1 peptide delayed, but did not prevent, Htt^{Q46} aggregation. To test this hypothesis, we analyzed Htt^{Q46} aggregation in the presence or absence of the SRCP1 peptide after a 72-hour incubation. Consistent with the SRCP1 peptide delaying, but not preventing aggregation, Htt^{Q46} aggregates were detected by EM and filter trap analysis (FIG. 8L, M) and the smaller species observed via DLS at 5 hours were no longer detectable at 72 hours (FIG. 8N). To further analyze SRCP1's pseudo-amyloid domain in vitro, we tested peptides of the two predicted individual domains (amino acids 61-70 or 71-80) within the SRCP1 peptide. Consistent with our cell data, a peptide that encodes amino acids 61-70 was sufficient to suppress Htt^{Q46} aggregation in vitro whereas a peptide that consists of amino acids 71-80 did not (FIG. 8O). Similarly, a peptide consisting of amino acids 61-70 with V65 and 169 mutated to alanine resulted in a loss in activity in accordance with the fluorescent microscopy data in cells (FIG. 8G, P). Together, these data support a role for SRCP1's C-terminal pseudo-amyloid domain in contributing to SRCP1's ability to suppress polyQ aggregation.

SRCP1 Reverses Neurite Shortening in Huntington's Disease (HD) iPSC-Derived Neurons

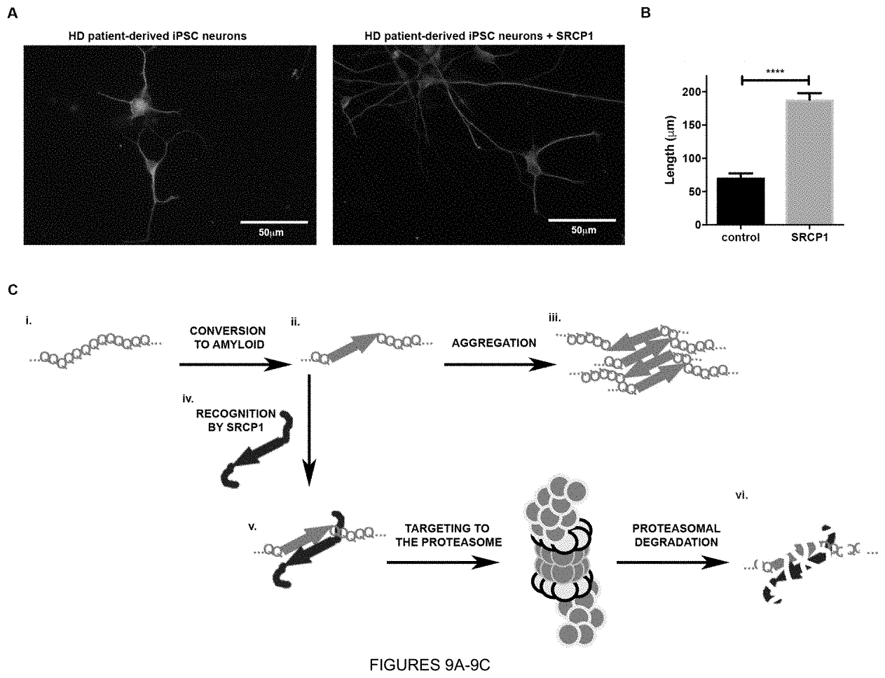

Because SRCP1 suppressed polyQ aggregation, we next wanted to determine if SRCP1 reversed disease phenotypes associated with huntingtin aggregation. One such phenotype is the degeneration of neurons, which is believed to contribute to the early pathology of Huntington's disease (DiFiglia et al., 1997; Li et al., 2001). To this end, we utilized two independent HD iPSC-derived neurons with either 60 or 180 glutamines (HD iPSC Consortium, 2012). Neurons derived from HD iPSCs exhibit shortened neurites (Chae et al., 2012; Kaye and Finkbeiner, 2013; The Hd iPsc Consortium, 2012), so we tested if SRCP1 could reverse this phenotype. We expressed ^{RFP}SRCP1 in HD iPSC-derived neurons and quantified the length of the neurites in the presence or absence of ^{RFP}SRCP1. Overexpression of SRCP1 significantly increased neurite length and reversed the phenotype seen in the untreated HD iPSC-derived neurons (FIG. 9A, B). This is consistent with SRCP1 preventing aggregation-related phenotypes and suggests that SRCP1 may prevent toxic events associated with human HD.

### DISCUSSION

The model organism, Dictyostelium discoideum, is a proteostatic outlier that naturally encodes for a large number of homopolymeric amino acid tracts. Among the repeat sequences encoded in the Dictyostelium genome, polyQ is among the most abundant, and the length of these polyQ repeats can reach well within the disease range in humans (Eichinger et al., 2005). Here we have identified a novel chaperone, SRCP1, that suppresses polyQ aggregation in Dictyostelium (FIG. 2). We show that SRCP1 is sufficient to suppress polyQ aggregation in multiple systems, including Dictyostelium, HEK293 cells, iPSC-derived human neurons, and zebrafish spinal cord neurons (FIG. 3). SRCP1 accomplishes this by selectively recognizing aggregation-prone, polyQ-expanded proteins and targeting them to the proteasome for degradation (FIG. 5D-F). Intriguingly, SRCP1 does not alter levels of soluble polyQ-expanded protein, indicating that it discriminates between soluble and aggregation-prone, polyQ (FIG. 3). In addition to targeting polyQ-expanded proteins to the proteasome, SRCP1 is also degraded by the proteasome and its degradation is enhanced by the presence of polyQ-expanded protein (FIG. 5, 6). We also observed that SRCP1 suppressed polyQ aggregation in the presence of both proteasome and autophagy inhibition, indicating a chaperone function for SRCP1 (FIG. 5, 6). We show that SRCP1's serine-rich N-terminus is dispensable for SRCP1 activity in cells (FIG. 7). However, we did identify a pseudo-amyloid domain in SRCP1's C-terminus that is necessary for SRCP1's ability to suppress polyQ aggregation (FIG. 8D-G). Finally, we show that expression of SRCP1 in Huntington's disease (HD) iPSC-derived neurons rescues defects in neurite outgrowth (FIG. 9A, B). Together, our findings identify a new type of chaperone that effectively prevents the accumulation of polyQ aggregates (FIG. 9C) and provides insight into how nature has dealt with the problem of polyQ aggregation.

SRCP1 is a Dictyostelium discoideum Specific Protein

While no previous reports have analyzed SRCP1 function and SRCP1 does not have any readily recognizable domains, our data indicate it functions as a novel type of molecular chaperone. SRCP1 is a member of a large class of Dictyostelium discoideum genes that encode small proteins (˜6-11 kDa) with serine-rich regions that have been implicated in Dictyostelium's developmental process (Vicente et al., 2008). This suggests that Dictyostelium's developmental process may have led to proteins and pathways that allow Dictyostelium to suppress aggregation of its repeat-rich proteome. Unlike most of these genes, SRCP1 levels are not sharply developmentally regulated and instead, SRCP1 is expressed throughout the Dictyostelium life cycle (Scaglione, unpublished results). In the future, more work is needed to understand the function of other members of this gene family to determine if they play similar roles to SRCP1 in suppressing protein aggregation. Because Dictyostelium encode for amino acid repeats for every amino acid except tryptophan, it will be important to expand studies beyond polyQ.

SRCP1 is a Novel Type of Molecular Chaperone

SRCP1 does not contain any readily identifiable chaperone domains. Instead we identified a C-terminal domain in SRCP1 that resembles amyloid. This pseudo-amyloid domain is necessary for SRCP1's chaperone activity in cells (FIGS. 8D-G), and peptides that mimic its C-terminus alter the rate of amyloid formation (FIG. 8H-P). Previous work identified that peptides that efficiently integrated into amyloid fibers (Cheng et al., 2012; Eskici and Gur, 2013; Sato et al., 2006) served as potent inhibitors of amyloid formation, suggesting that SRCP1's pseudo-amyloid domain may bind ^{GFP}Htt^{ex1Q74} once it has converted to an aggregation-prone amyloid fold. In the future, more work is needed to fully understand the mechanism by which SRCP1 suppresses polyQ aggregation.

In addition to SRCP1's chaperone activity, SRCP1 also targets ^{GFP}Htt^{ex1Q74} for proteasomal degradation, and SRCP1 itself is also degraded by the proteasome. However, the mechanism by which SRCP1 and polyQ are targeted to the proteasome is unclear. Proteasomal substrates are typically targeted to the proteasome via ubiquitination (Kwon and Ciechanover, 2017). However, it is unclear if SRCP1 targets ^{GFP}Htt^{ex1Q74} for ubiquitination or is ubiquitinated itself. Alternatively, some proteins, like the neurodegenerative disease protein tau, are degraded in the absence of ubiquitination (Blair et al., 2013; David et al., 2002; Grune et al., 2010). This raises the possibility that SRCP1 may target ^{GFP}Htt^{ex1Q74} for degradation independent of ubiquitin signaling. In the future the identification of members of the ubiquitin proteasome system that facilitate SRCP1 function will be important.

While there remains some debate about whether large aggregates observed in Huntington's disease are toxic or protective, it is increasingly clear that smaller oligomeric huntingtin species are toxic (Hoffner and Djian, 2014). In addition to smaller oligomeric species causing toxicity, monomeric polyQ has also been shown to cause toxicity in cells (Nagai et al., 2007). Interestingly, the toxicity of this monomeric polyQ protein is confirmation dependent as only polyQ that has adopted a [symbol] -sheet confirmation induces toxicity in cells (Nagai et al., 2007). This is interesting because SRCP1's pseudo-amyloid domain is necessary for its ability to prevent polyQ aggregates in cells (FIGS. 8D-G) and a peptide that mimics this domain can delay the formation of polyQ aggregates in vitro (FIG. 8H-P). As a conformational change to a [symbol] -sheet-rich structure is an integral component of amyloid formation, we hypothesize that SRCP1's pseudo-amyloid domain forms mixed amyloid and selectively identifies monomeric polyQ that has adopted this [symbol] -sheet confirmation. This would allow SRCP1 to selectively identify this monomeric, aggregation-prone polyQ protein. Consistent with this we see that SRCP1 has no effect on the levels of ^{GFP}Htt^{ex1Q23} or soluble ^{GFP}Htt^{ex1Q74} suggesting SRCP1 can discriminate between properly folded and misfolded polyQ (FIG. 3D-F, J,K). Additionally, we observe that SRCP1 targets aggregation-prone ^{GFP}Htt^{ex1Q74} to the proteasome for degradation (FIG. 5D-F). This further supports the notion that SRCP1 is selectively targeting the monomeric version of ^{GFP}Htt^{ex1Q74} as the proteasome is inefficient at degrading aggregated polyQ protein (Holmberg et al., 2004).

SRCP1 Provides Insight into Therapies

SRCP1's unique ability to selectively discriminate between soluble and aggregation-prone ^{GFP}Htt^{ex1Q74} (FIG. 3, 5) has important therapeutic implications. For example, huntingtin has essential roles in development (Duyao et al., 1995; Nasir et al., 1995; Zeitlin et al., 1995), and ataxin-3, the polyQ protein that causes Spinocerebellar ataxia type 3, plays an important role in autophagy (Ashkenazi et al., 2017). The development of molecules that selectively identify and clear aggregation-prone, polyQ-expanded proteins may be a better therapeutic avenue.

Methods

Experimental Model and Subject Details

Mammalian Cell Culture

Human embryonic kidney293 (HEK293) cells were grown at 37° C. and 5.2% CO₂. HEK293 cells were maintained in Dulbecco's Modified Eagle's Medium (Gibco by Life Technologies) supplemented with 10% fetal bovine serum (Atlanta biologicals) and 1% Penicillin-Streptomycin (Gibco by Life technologies).

Dictyostelium discoideum Cell Culture

Dictyostelium discoideum AX4 cells were maintained in shaking cultures at 22° C. in HL5 (17.8 g peptone, 7.2 g yeast extract, 0.54 g Na₂HPO₄, 0.4 g KH₂PO₄, 130 μl B12/Folic acid, 20 ml of 50% w/v glucose, ampicillin 100 μg/ml, pH 6.5) media. Cells were maintained at a density no greater than 6×10⁶ cells/ml. For growth on bacteria, Dictyostelium cells between a density of 1×10⁶-6×10⁶ cells/ml, were diluted 1:100, 1:1000, and 1:10,000. Approximately, 500 μl of dilutions were spread on bacterial plates. To prepare bacterial plates, K. aerogenes (Dictybase) was grown at room temperature for two days and then plated on freshly made SM plates (35 ml per 100 mm Petri dish; 1 Liter: 10 g glucose, 10 g proteose peptone, 1 g yeast extract, 1 g MgSO₄*7H₂O (or 0.5 g MgSO₄), 1.9 g KH₂PO₄, 0.6 g K₂HPO₄, 20 g Agar) and incubated overnight at room temperature.

Bacterial Cell Culture

To obtain plasmid DNA, overnight bacterial cultures were grown overnight at 37° C. with a selection antibiotic at 50 μg/ml. One Shot TOP10 chemically competent E. coli (Invitrogen by ThermoFisher Scientific) cells were used for any cloning procedures. For all other plasmids XL-10 E. coli cells were used. All E. coli cells were stored at −80° C. until necessary.

iPSC Culture

Induced pluripotent stem cells (iPSCs) used were karyotypically normal and mycoplasma negative. iPSCs were cultured in T25 ultra-low attachment culture flasks (Corning) as non-adherent neural progenitor cell aggregates in Stemline medium (Sigma-Aldrich) supplemented with 100 ng/ml epidermal growth factor (Miltenyi), 100 ng/ml fibroblast growth factor (Stem Cell Technologies), 5 μg/ml heparin (Sigma-Aldrich), and 0.5% N2 (Life Technologies) in humidified incubators at 37° C. and 5.0% CO₂. Subsequently, neural progenitor cells were differentiated into neurons and astrocytes as previously described (Ebert et al., 2013). Briefly, cells were dissociated with TrypLE (Life Technologies) and seeded onto Matrigel (Corning) coated glass coverslips at 2.6×10⁴ cells/cm². For differentiation, GFAP+ astrocytes and Tuj1+ neurons were grown in Neurobasal medium (Life Technologies) supplemented with 2% B27 (Life Technologies) and Antibiotic-Antimycotic (Life Technologies) for 1-2 weeks. Following differentiation, iPSC-derived astrocytes exhibit functional calcium responses to ATP (McGivern et al., 2013) and potassium currents (Ebert et al., 2013), and iPSC-derived neurons exhibit NR2B NMDA receptor expression (Schwab et al., 2017) and appropriate electrophysiological properties (2012; The Hd iPsc Consortium, 2012)

Zebrafish Colony Management

Zebrafish were housed in a closed circulating system using water purified by reverse osmosis, and subjected to 10% daily flush. Conductivity was maintained at 800 μS. Particulates were removed by drum filtration. The light:dark cycle was 14L:10D. Fish were fed three times per day with hatched artemia. For experiments, embryos derived from group crosses of the ZDR strain were used.

Method Details

Expression Constructs

For Dictyostelium expression huntingtin exon-1 with 103 glutamines was cloned into pTX-GFP (Dictybase) using KpnI and XbaI (^{GFP}Htt^{ex1Q103}). For restriction-enzyme mediated integration, the kanamycin resistance cassette was swapped in place of the ampicillin resistance cassette using BamHI and SpeI sites in the pDM323 (Dictybase) vector resulting in a kanamycin resistant pDM323 (pDM323 KAN) plasmid. ^{GFP}Htt^{ex1Q103} with 103 glutamines was cloned into the generated pDM323 KAN plasmid. The pBSR3 plasmid (Dictybase) was used for the integration step. SRCP1 (DDB_G0293362), SRCP1 with amino acids 61-70 or 71-80 mutated to alanine, and SRCP1 with serine and threonine mutated to alanine were synthesized for mammalian expression into the PS100049 vector by Blue Heron. Individual point mutants of SRCP1 61-70 were generated using QuikChange Lightning Site-Directed Mutagenesis Kit (Agilent). Plasmids for mammalian expression encoding pEGFP huntingtin exon-1 with 23 (^{GFP}Htt^{ex1Q23}) or 74 (^{GFP}Htt^{ex1Q74}) glutamines were obtained from Addgene.

Dictyostelium Cell Transformation

Transformations were performed via electroporation as described previously (Knecht and Pang, 1995). Briefly, 5×10⁶ cells were spun down at 500 g for 5 minutes at 4° C. Cells were then washed three times with ice-cold H-50 buffer (20 mM HEPES, 50 mM KCl, 10 mM NaCl, 1 mM MgSO₄, 5 mM NaHCO₃, 1 mM NaH₂PO₄), resuspended in 100 μl of ice-cold H-50 buffer, and combined with 10 μg of DNA. Cell and DNA mixture was added to a pre-cooled 1 mm cuvette and electroporated at 0.85 kV/25 μF/time constant 0.6 msec twice with about 5 seconds between pulses. Cells were then incubated on ice for 5 minutes and added to 10 ml of HL5 media on 10 cm plates. Appropriate selection drugs were added the following day and included in media going forward. Selection drugs include G-418 (Gibco by Life technologies) at 10 μg/ml, blasticidin (GoldBio) at 10 μg/ml (except where indicated), and hygromycin B (ThermoFischer Scientific) at 50 μg/ml.

Restriction Enzyme-Mediated Integration (Kuspa, 2006)

10 μg of the pBSR3 construct was linearized with BamHI, electroporated into AX4 cells along with 50 units of DpnII, and selected for one week with blasticidin at 4 μg/ml. Following selection, pBSR3 cells were electroporated with pDM323(Kan)^{GFP}Htt^{ex1Q103} construct and selected for one week with blasticidin at 4 μg/ml and G-418 at 10 μg/ml. To obtain individual colonies, cells were then plated on SM bacterial plates in serial dilutions. This was performed until the number of clones desired was obtained. Upon the appearance of Dictyostelium colonies on the bacterial plates, colonies were picked and grown in HL5 media in 96-well plates. Once cells had reached confluency, 96-well plates were washed with ice-cold starvation buffer (0.1 M MES, 0.2 mM CaCl₂, 2 mM MgSO₄, pH 6.8) twice and screened for aggregates using the ArrayScan High-Content Imaging System (ThermoScientific). Cells with greater than 5% aggregates were considered “hits” and grown up in 10 cm plates.

For gene identification, genomic DNA was first isolated from Dictyostelium “hit” clones. Genomic DNA was then digested with either ClaI, HindII, or BgIII for an hour at 37° C., purified and ligated for 30 minutes at room temperature. Ligated DNA was then transformed into One Shot TOP10 chemically competent E. coli. Bacterial colonies were screened by restriction digest for insert and then sent for sequencing.

Dictyostelium Knockout Generation

Dictystelium knockout vectors were generated following the StarGate® Acceptor Vector pKOSG-IBA-Dictyl system (iba-lifesciences) (Wiegand et al., 2011). The knockout vector was then linearized, electroporated into AX4 cells, and selected for one week with blasticidin at 10 μg/ml. To isolate clones, the electroporated cells were plated on SM bacterial plates. Individual colonies were then picked and grown up to confluency in 10 cm dishes. To screen knockouts, genomic DNA was obtained and utilized in PCR to confirm blasticidin insertion as well as flanking regions of SRCP1. To confirm SRCP1 knockouts, clones were electroporated with ^{GFP}Htt^{ex1Q103} and selected with G-418 for one week at 10 μg/ml. Cells were then imaged by fluorescent microscopy and analyzed for ^{GFP}Htt^{ex1Q103} aggregates.

Differential Centrifugation in Dictyostelium

For differential centrifugation, 1×10⁷ cells were washed with 1×PBS and lysed with NETN ((0.5% NonidetP-40, 150 mM NaCl, 50 mM Tris, and protease inhibitor (GoldBio)). Samples were centrifuged at 15,000 rpm for 30 minutes at 4° C. The supernatant (soluble fraction) was removed and subjected to BCA protein assay. Remaining pellet (insoluble fraction) was washed three times with NETN, resuspended in 1× Laemmli buffer (4× stock: 40% Glycerol, 240 mM Tris/HCl pH 6.8, 8% SDS, 0.04% bromophenol blue, 5% beta-mercaptoethanol) and analyzed by western blot.

Genomic DNA Isolation in Dictyostelium (Pilcher et al., 2007)

Approximately, 3×10⁷ cells were pelleted by centrifugation for 4 minutes at 500 g at room temperature and washed twice with 1×PBS. Cells were resuspended in 1 ml of nuclei buffer (20 mM Tris-HCl pH 7.4, 5 mM MgOAc, 1 mM EDTA pH 8.0, 5% (w/v) sucrose, 1 mM EGTA) and lysed by the addition of 200 μl of 20% Triton X-100 and incubation on ice for 20 minutes. To obtain genomic DNA, lysates were centrifuged at 12,000 g for 5 min at 4° C. The supernatant was removed and pellet was resuspended in 300 μl of proteinase K Buffer (100 mM Tris-HCl pH 7.4, 5 mM EDTA pH 8.0, 0.1 mg ml⁻¹ proteinase K, 1% (v/v) SDS) and incubated at 65° C. for 30 min. Nucleic acids were extracted by adding an equal volume (300 μl) of phenol:chloroform (1:1). Samples were inverted to mix and centrifuged at 12,000 g for 20 minutes at 4° C. The aqueous (upper) layer was transferred into a fresh tube and mixed with one volume (300 μl) of chloroform by inversion. Samples were centrifuged at 12,000 g for 10 minutes at 4° C. The aqueous (upper) layer was transferred into a fresh tube and precipitated overnight at −20° C. with 2.5 volumes (750 μl) of ice-cold 100% ethanol. DNA was pelleted by centrifugation at 12,000 g for 30 minutes at 4° C. Supernatant was removed and pellet was resuspended in 100 μl of TE (10 mM Tris-HCl pH 7.4, 1 mM EDTA) containing 10 μg ml⁻¹ RNase A and incubated at room temperature for 15 minutes. DNA was precipitated again overnight at −20° C. by the addition 1/10 volume (10 μl) of 3 M NaOAc and 2.5 volumes (250 μl) of ice-cold 100% ethanol. DNA was pelleted by centrifugation at 12,000 g for 30 minutes at 4° C. The supernatant was removed and pellet was washed with two volumes (250 μl) of ice-cold 70% ethanol. DNA was pelleted by centrifugation at 12,000 g for 2 minutes at 4° C. Supernatant was removed and pellet was dried at room temperature for 10 minutes. DNA pellet was resuspended in 50 μl of TE pH 7.4 and stored at 4° C.

Chemical Inhibition in Dictyostelium

For proteasome inhibition, 5×10⁶ cells were incubated with either DMSO or 100 μM MG132 (Sigma-Aldrich) for 18 hours. Following treatment, cells were imaged and/or harvested by spinning at 500 g at room temperature for 5 minutes. Cell pellets were washed three times with 1×PBS and lysed with ice-cold NETN (with protease inhibitor). Lysates were then sonicated twice for 10-15 seconds. To verify inhibition, samples were run on SDS-PAGE and analyzed by western blot for ubiquitin.

For autophagy inhibition, 1×10⁷ cells were incubated either with vehicle or 150 μM NH₄Cl (Sigma-Aldrich) for 8 hours. Following treatments, cells were imaged by fluorescent microscopy and plated on coverslips at a density of 2×10⁶ cells per ml and allowing cells to adhere. Cells were then washed three times with 1×PBS and fixed with 100% ice-cold methanol at −20° C. for 10 minutes. Methanol was aspirated and cells were washed twice with 1×PBT (0.1% Triton X, 0.5% BSA in 1×PBS), incubated in blocking buffer (1% Triton X, 2% BSA in 1×PBT) for 30 minutes at room temperature, and put in primary Rb anti ATG8a (courtesy of Jason King, University of Sheffield) (1:5000) overnight at 4° C. Following primary cells were washed three times with 1×PBT and incubated in secondary goat anti rabbit (Jackson ImmunoResearch Laboratories; 711-166-152) for 2 hours at room temperature. Following secondary, cells were washed an additional three times with 1×PBT and mounted on slides with ProLong Gold Antifade reagent (Invitrogen by ThermoFisher Scientific). Slides were imaged with a Nikon eclipse 90i confocal microscope.

For Hsp70 inhibition, 1×10⁷ cells were incubated either with DMSO or 80 μM VER155008 (ApexBio) for 24 hours. Following treatments, cells were imaged and/or harvested by spinning at 500 g at room temperature for 5 minutes. Cell pellets were washed three times with 1×PBS and lysed with ice-cold NETN (with protease inhibitor). Lysates were then sonicated twice for 10-15 seconds. To verify inhibition, samples were run on SDS-PAGE and analyzed by western blot for ATG8.

Cell Viability Assay

HEK293 cells were transfected as described with RFP or ^{RFP}SRCP1. Following transfections, cells were diluted to 500,000 cells/mL with 50,000 cells plated per well in duplicate from each transfection reaction in a 96-well plate. Serial dilutions were then performed down each column of the plate down to 390 cells per well. Cells were cultured for an additional 24 hours, then lysed using the CellTiter-Glo Kit (Promega). Luminescence was quantified between 565-700 nm wavelengths on a Spark microplate reader (Tecan) to determine cell viability.

Mammalian Cell Transfections

Transfections were performed with Lipofectamine 2000 (Invitrogen by ThermoFisher Scientific) and adapted from the manufacturer's instructions. Briefly, cells were plated on either 12-well or 6-well plates and transfected at 50-70% confluency. For 12-well transfections, 0.833 μg of DNA per well was mixed with 50 μl of OptiMEM (Gibco by Life Technologies) media and incubated for 5 minutes at room temperature. Lipofectamine 2000 in a 2:1, μl of lipofectamine: μg DNA ratio was mixed with 50 μl of OptiMEM media and incubated for 5 minutes at room temperature. DNA and Lipofectamine were mixed and incubated for 15 minutes at room temperature. Fresh media was added to cells prior to the addition of DNA and lipofectamine. For 6-well transfections, the same protocol is followed except that 1.25 μg of DNA per well and 100 μl of OptiMEM for dilution was used. Media was changed 24 hours post-transfection and cells were harvested 48 hours post-transfection. Prior to harvesting, HEK293 cells were washed three times with ice-cold 1×PBS. Samples were then lysed with either 150 μl of ice-cold NETN (with protease inhibitor) or 1× Laemmli Buffer and sonicated twice for 5-8 seconds.

iPSC transfections were performed with mixed cultures of astrocytes and neurons adhered to matrigel-coated coverslips, seeded at 2.6×10⁴ cells/cm². iPSC transfections were performed using Lipofectamine 2000 according to the manufacturer's instructions and recommended DNA concentrations for a 24-well transfection.

Chemical Inhibition in HEK293 Cells

For proteasome inhibition, 30 hours post-transfection cells would be replenished with DMEM containing either DMSO or 10 μM MG132 (Sigma-Aldrich) for 18 hours. Prior to harvesting, HEK293 cells were washed three times with ice-cold 1×PBS and imaged by fluorescent microscopy with a 20× objective using the Evos FL Auto Imaging System. Cells were then lysed with either 150 μl of ice-cold NETN (with protease inhibitor) or 1× Laemmli Buffer and sonicated twice for 5-8 seconds.

For autophagy inhibition, 24 hours post-transfection cells would be replenished with DMEM and treated with either DMSO or 5 mM 3-Methyladenine (Sigma-Aldrich) for an additional 24 hours. Prior to harvesting, HEK293 cells were washed three times with ice-cold 1×PBS and imaged by fluorescent microscopy with a 20× objective using the Evos FL Auto Imaging System. Cells were then lysed with either 150 μl of ice-cold NETN (with protease inhibitor) or 1× Laemmli Buffer and sonicated twice for 5-8 seconds.

Microscopy

For HEK293s′, slides were prepared by placing coverslips in 6-well plates and incubating with poly-L-lysine hydrobromide (Sigma-Aldrich) for an hour at room temperature. Coverslips were rinsed with 1×PBS three times prior to plating HEK293 cells. HEK293 cells were then transfected and fixed by incubation in 4% paraformaldehyde for 20 minutes at room temperature. Coverslips were mounted with ProLong Gold Antifade reagent and imaged with a 20× objective using the Evos FL Auto Imaging System or with a 40× objective using the Leica TCS SP5 Confocal Microscope System. For confocal, Z-stack images were obtained at 0.5-m intervals at 1024×1024 pixel resolution and merged using Fiji.

Plated iPSC derived cultures of astrocytes and neurons were fixed with 4% paraformaldehyde in PBS for 20 minutes at room temperature, 3-5 days after transfection. Cells were permeabilized and blocked prior to antibody labeling. Nuclei were labeled with Hoechst nuclear dye. Primary antibodies used were rabbit anti-Tuj1 (Covance MRB-435P), chicken anti-Tuj1(Gene Tex, GTX85469) and mouse anti-GFAP (Cell Signaling, 3670). Images were taken using an upright Nikon fluorescent microscope with a 40× objective. The number of neural cells with GFP positive puncta was quantified using NIS Elements object quantification tool (Nikon) and neurite length was measured using NIS Elements length measurement tool (Nikon).

For Dictyostelium cells, 1×10⁷ cells were washed three times with ice-cold starvation buffer and then imaged with a 20× objective using the Evos FL Auto Imaging System.

Zebrafish Analysis

To test the effect of ^{RFP}SRCP1 on ^{GFP}Htt^{ex1Q74} aggregation in zebrafish, each plasmid was used for in vitro synthesis of mRNA (mMessage mMachine T7 transcription kit, ThermoFisher Scientific). Newly fertilized embryos were injected with either 200 pg mRNA each of ^{RFP}SRCP1 and ^{GFP}Htt^{ex1Q74}, or RFP alone and ^{GFP}Htt^{ex1Q74}. Embryos were developed until 54 hours post fertilization, at which time live specimens were anesthetized in Tricane, embedded in 1% low-melt agarose, and subjected to confocal microscopy. Spinal cord images were taken at 100 μm×100 μm and a scan depth of 50 μm. Single image planes from 488 nm and 568 nm excitation were collected. Images were then processed for threshold analysis using ImageJ and the total number puncta were scored.

SDS-PAGE and Western Blot

Following sonication, lysates were subjected to BCA protein assay (ThermoFisher Scientific) and prepared for loading by the addition of 4× laemmli buffer and boiling for four minutes. Samples then loaded on SDS-polyacrylamide gels, ran at 175 V, and transferred onto an Immuno-blot PVDF membrane (Biorad) overnight at 30 V. Membranes were blocked in 5% milk in TBS with 0.1% Tween (TBST) and incubated in primary antibody overnight at 4° C. Following primary, membranes were washed for 10 minutes at room temperature three times with TBST and incubated in secondary for an hour at room temperature. Membranes were then washed for 10 minutes at room temperature three times with TBST and then incubated in buffer for enhanced chemiluminescence (50 mM Na₂HPO₄; 50 mM Na₂CO₃; 10 mM NaBO3.4H₂O; 250 mM luminol; 90 mM coumaric acid).

Anti-GFP (Invitrogen by ThermoFisher Scientific; A11122), anti-RFP (Invitrogen by ThermoFisher Scientific; MA5-15257), anti-polyglutamine-expansion (EMD Millipore; MAB1574), and anti-ubiquitin (BD Pharmingen; 550944) were used at 1:1000. Peroxidase-conjugated secondary antibodies (Jackson ImmunoResearch Laboratories; 111-035-045; 115-035-174) were used at 1:5,000 dilution. Anti-3-Actin was used as a loading control at 1:1000 (Invitrogen by ThermoFisher Scientific, PA121167).

Filter Trap Assay

For HEK293 lysates, BCA protein assay was used to determine protein concentration. Forty micrograms of protein were diluted up 90 μl with NETN (with protease inhibitor) 10 μl of 10% SDS was added to protein sample and diluted to 1 ml with 1% SDS in 1×PBS, and filtered through a 0.2 mm cellulose acetate membrane filter (Sterlitech) using a DHM-48 filter trap hybridization manifold. Membrane was then washed with 1 ml of 1% SDS in 1×PBS and analyzed by western blotting (Scherzinger et al., 1999b; Wanker et al., 1999).

Huntingtin Exon-1 46Q Purification

Huntingtin exon-1 with 46 glutamines (Htt^{Q46}) in pET-32 was obtained from Addgene. Htt^{Q46} was grown in BL21 cells at 37° C. to an optical density of 0.6 and induced with IPTG at 1 mM overnight at 16° C. After induction cells were spun down at 7,000 rpm for 5 minutes and resuspended in resuspension buffer (15 mM Tris-HCl buffer, pH 8.0). For lysis, lysozyme was added and cells were incubated at 4° C. for 45 minutes. To obtain soluble fraction, lysates were spun down at 12,000 rpm for 10 minutes, after which supernatant was added to 3 ml of Ni-beads (GoldBio) per 100 ml of lysate and tumbled for 4 hours at 4° C. Beads were then washed three times with resuspension buffer and then washed three more times with wash buffer (50 mM Tris-HCl pH 8.0, 150 mM NaCl, 1 mM PMSF, 1 mM EDTA). Protein was then eluted off beads by tumbling overnight at 4° C. in 25 mL of wash buffer with 250 mM imidazole.

Thioflavin Aggregation Assays

Aggregation assays were performed as previously described in Kakkar, V. et al. Briefly, 15 μM Htt⁴⁶Q was mixed with 90 μM SRCP1 peptide (GenScript) (1:6, 1:3, 1:1.5) and 10 μM Thioflavin-T (Sigma-Aldrich). All samples were prepared on ice in buffer containing 20 mM Tris-HCl pH 8.0, 50 mM NaCl, 2 mM CaCl₂). Enterokinase (New England BioLabs) was added at 1.6 units/sample to initiate aggregation. All samples were prepared on ice and 50 μL aliquots were transferred to a flat, black 384-well plate and allowed to aggregate at 37° C. for 16-18 hours. Fluorescence was measured with the excitation at 440 nm and emission at 480 nm, every 5 minutes using a Tecan Plate reader.

Filter Trap Analysis of Recombinant Protein

For filter trap assays, Htt⁴⁶Q was used at a concentration of 15 μM alone or with 90 μM of SRCP1 peptide. Peptide alone was used at 50 μM. All samples were prepared on ice in buffer containing 20 mM Tris-HCl pH 8.0, 50 mM NaCl, 2 mM CaCl₂). Enterokinase at 1.6 units/sample was added to initiate the reactions and samples were allowed to aggregate at 37° C. for 5 and 72 hours. Approximately 40 μg of protein sample were then used for filter trap assay.

Dynamic Light Scattering (DLS)

For dynamic light scattering (DLS) measurements, Htt⁴⁶Q was used at a concentration of 15 μM alone or with 90 μM of SRCP1 peptide. Peptide alone was used at 50 μM. All samples were prepared on ice in buffer containing 20 mM Tris-HCl pH 8.0, 50 mM NaCl, 2 mM CaCl₂). Enterokinase at 1.6 units/sample was added to initiate the reactions and samples were allowed to aggregate at 37° C. for 5 and 72 hours. Following aggregation, 10 μL of sample was loaded into a Hellma Analytics QC High Precision Cell Quartz Suprasil Cuvette (cuvette model # ZMV1002, light path of 1.25×1.25 mm). DLS measurements were collected at 25° C. by using a Malvern Zetasizer μV with a 50 mW laser at 830 nm, using a detector angle of 90⁻. The laser power was set to 30% power and integration time was set to 100 seconds. Each measurement consisted of 50-60 collections and are representative of n=2, where each n consisted of at least 2 technical replicates. DLS spectra were analyzed and visualized in R (Team, 2016) by using the tidyverse (Wickham, 2017), scales (Wickham, 2016), and directlabels (Hocking, 2017) packages.

Electron Microscopy (EM)

For EM, samples were prepared on ice in buffer containing 20 mM Tris-HCl pH 8.0, 50 mM NaCl, 2 mM CaCl₂). Htt⁴⁶Q was used at a concentration of 30 μM alone or with 174 μM of SRCP1 peptide. Peptide alone was used at 50 μM. Enterokinase at 2.2 units/sample was added to initiate the reactions and samples were allowed to aggregate at 37° C. for 5 and 72 hours.

Following aggregation assays, freshly ionized 400 mesh Formvar/carbon coated copper grids were floated onto 10 μl droplets of sample and stain for 2 minutes to allow adsorption of the sample to the formvar/carbon film. After adsorption, the sample was then wicked away from the edge of grid surface and the grid was immediately floated on the droplet of negative stain (2% aqueous uranyl acetate) for 1 minute. The stain was wicked away from the edge of the grid and the grid was then allowed to air dry. Samples were examined in a Hitachi H600 TEM.

Quantification and Statistical Analysis

For the high-content imaging, HCS program was used to develop custom algorithms to detect puncta and number of cells. Using the two algorithms, the HCS program quantified the percent of cells with aggregates.

For general microscopy analysis, ImageJ was used to quantify the number of cells per puncta and the puncta size where indicated. ImageJ was also used for the quantification of western blots. Briefly, band of interest would be measured for intensity and normalized to the intensity of the loading control.

Values were then entered in Graph Pad where they were analyzed by either an ANOVA for multiple comparisons and Students t-tests for 1:1 comparisons followed by Tukey's post-hoc analysis. Differences were considered statically significant at a p-value less than 0.05.

For control iPSCs, four biological replicates from two independent differentiations were analyzed and for HD iPSCs, four biological replicates were analyzed from four separate differentiations using two independent HD lines.

KEY RESOURCES TABLE

| REAGENT or RESOURCE | SOURCE | IDENTIFIER |
|---|---|---|
| Antibodies | | |
| Anti-Actin, rabbit polyclonal | ThermoFisher | PA1-21167; |
| | Scientific | RRID:AB_557422 |
| Anti-Atg8a, rabbit polyclonal | Jason King | pAb 7141 |
| Anti-GFAP, mouse monoclonal | Cell Signaling | 3670; |
| | | RRID:AB_561049 |
| Anti-GFP, rabbit polyclonal | ThermoFisher | A-11122; |
| | Scientific | RRID:AB_221569 |
| Anti-Polyglutamine-Expansion Diseases Marker | EMD Millipore | MAB1574; |
| Antibody, clone 5TF1-1C2 | | RRID:AB_94263 |
| Anti-RFP, mouse monoclonal (RFSR) | ThermoFisher | MAS-15257; |
| | Scientific | RRID:AB_109997 |
| | | 96 |
| Anti-Tuj1, chicken | Gene Tex | GTX85469; |
| | | RRID:AB_106292 |
| | | 2 |
| | | 2 |
| Anti-TUJ1, rabbit monoclonal | Covance | MRB-435P |
| Anti-ubiquitin, mouse monoclonal (Clone 6C1.17) | BD Biosciences | 550944 |
| Donkey anti Chicken | Jackson | 703-604-155 |
| | ImmunoResearch | RRID:AB_234037 |
| | | 5 |
| Donkey anti Mouse | Jackson | 715-165-150 |
| | ImmunoResearch | RRID:AB_234081 |
| | | 3 |
| Donkey anti Rabbit | Jackson | 711-165-152 |
| | ImmunoResearch | RRID:AB_230744 |
| | | 3 |
| Donkey anti Rabbit IgG | Jackson | 711-166-152 |
| | ImmunoResearch | RRID:AB_231356 |
| | | 8 |
| Goat anti Mouse HRP | Jackson | 115-035-174; |
| | ImmunoResearch | RRID:AB_233851 |
| | | 2 |
| Goat anti Rabbit HRP | Jackson | 111-035-045; |
| | ImmunoResearch | RRID:AB_233793 |
| | | 8 |
| Bacterial and Virus Strains | | |
| Klebsiella aerogenes | Dictybase | DB50349838 |
| One Shot TOP10 chemically competent E.coli | ThermoFisher | C404010 |
| | Scientific | |
| BL21(DE3) chemically competent E.coli | Novagen | 69450 |
| Chemicals, Peptides, and Recombinant Proteins | | |
| 17-AAG | Sigma | A8476 |
| Blasticidin | GoldBio | B-800-1 |
| Enterokinase | New England | P8070S |
| | BioLabs | |
| G-418 | ThermoFisher | 10131027 |
| | Scientific | |
| HttQ46 | This Study | N/A |
| Hygromycin B | ThermoFisher | 10687010 |
| | Scientific | |
| Lipofectamine 2000 | ThermoFisher | 11668 |
| | Scientific | |
| 3-MA | Sigma | M9281 |
| MG-132 | Sigma | M7449 |
| NH₄Cl | Sigma | A9434 |
| Poly-L-lysine hydrobromide | Sigma | P6282 |
| ProBlock ™ Gold mammalian protease | Gold Biotechnology | GB-331-5 |
| inhibitor cocktail | | |
| ProLong Gold Anti Fade Reagent | ThermoFisher | P10144 |
| | Scientific | |
| Thioflavin T | Sigma | T3516 |
| VER155008 | ApexBio | A4387 |
| SRCP1 Peptide Amino Acids 61-80 | GenScript | N/A |
| SRCP1 Peptide Amino Acids 61-70 | GenScript | N/A |
| SRCP1 Peptide Amino Acids 71-80 | GenScript | N/A |
| SRCP1 Peptide Amino Acids 61-70 V65, I69A | GenScript | N/A |
| Critical Commercial Assays | | |
| Pierce BCA Protein Assay Kit | ThermoFisher | 23225 |
| | Scientific | |
| CellTiter-Glo Kit | Promega | G7570 |
| Experimental Models: Cell Lines | | |
| Human Embryonic Kidney Cells | ATCC | CRL-1573; |
| | | RRID:CVCL_0045 |
| Control hiPSC | Allison Ebert | |
| | “Schwab, et al Stem | |
| | Cell Reports 2015” | |
| HD180.5 | Allison Ebert “HD | |
| | iPSC Consortium, | |
| | Cell Stem Cell | |
| | 2012” | |
| HD60i | Allison Ebert “HD | |
| | iPSC Consortium, | |
| | Cell Stem Cell | |
| | 2012” | |
| Experimental Models: Organisms/Strains | | |
| Dictyostelium discoideum AX4 | Dictybase | DBS0237637 |
| Dictyostelium discoideum AX4 SRCP1 Knockout | This study | N/A |
| Recombinant DNA | | |
| Plasmid: Huntingtin exon1Q23 in pEGFP-C1 | Addgene | 40261 |
| Plasmid: Huntingtin exon1Q74 in pEGFP-C1 | Addgene | 40262 |
| Plasmid: pCMV6-AN-mRFP | Addgene | PS100049 |
| Plasmid: SRCP1 in pCMV6-AN-mRFP | Blue Heron | N/A |
| Plasmid: SRCP1 ST → A in pCMV6-AN-mRFP | Blue Heron | N/A |
| Plasmid: SRCP1 61-70 → A in pCMV6-AN-mRFP | Blue Heron | N/A |
| Plasmid: SRCP1 71-80 → A in pCMV6-AN-mRFP | Blue Heron | N/A |
| Plasmid: SRCP1 V65 → A in pCMV6-AN-mRFP | This study | N/A |
| Plasmid: SRCP1 I69 → A in pCMV6-AN-mRFP | This study | N/A |
| Plasmid: Huntingtin exon1Q103 in pTxGFP | Dr. Scaglione | Santarriaga et al., |
| | | 2015 |
| Plasmid: pTxGFP | Dictybase | 11 |
| Plasmid: pBSR3 | Dictybase | 33 |
| Plasmid: pDM323 | Dictybase | 540 |
| Plasmid: pDM449 | Dictybase | 548 |
| Plasmid: pDM323 with kanamycin resistance | This study | N/A |
| Plasmid: pKOSG-IBA-Dicty1 | Iba-lifesciences | 5-1650-005 |
| Plasmid: SRCP1 Knockout in pKOSG-IBA-Dicty1 | This study | N/A |
| Plasmid: SRCP1 in pDM449 | This Study | N/A |
| Plasmid: Htt46Q in pET-32 | Addgene | 11515 |
| Software and Algorithms | | |
| Fiji | Open Source | Schindelin et al., |
| | | 2012 |
| GraphPad Prism Version 7.00 | GraphPad | https://www.gra |
| | | phpad.com/ |
| | | scientific- |
| | | software/prism/ |
| HSC | ThermoScientific | N/A |
| Malvern Instruments Zetasizer Software Version | Malvern | N/A |
| 7.11 (copyright 2002-2014) | | |
| R - Version 3.3.1 (2016 Jun. 21) | R Core Team | https://www.R- |
| | | project.org |
| RStudio - Version 1.1.383 (copyright 2009-2017) | R Core Team | https://www.R- |
| | | project.org |

### REFERENCES

(2012). Induced pluripotent stem cells from patients with Huntington's disease show CAG-repeat-expansion-associated phenotypes. Cell stem cell 11, 264-278.
Adegbuyiro, A., Sedighi, F., Pilkington, A. W. t., Groover, S., and Legleiter, J. (2017). Proteins Containing Expanded Polyglutamine Tracts and Neurodegenerative Disease. Biochemistry 56, 1199-1217.
Ashkenazi, A., Bento, C. F., Ricketts, T., Vicinanza, M., Siddiqi, F., Pavel, M., Squitieri, F., Hardenberg, M. C., Imarisio, S., Menzies, F. M., et al. (2017). Polyglutamine tracts regulate beclin 1-dependent autophagy. Nature 545, 108-111.
Bates, G. P., and Davies, S. W. (1997). Transgenic mouse models of neurodegenerative disease caused by CAG/polyglutamine expansions. Mol Med Today 3, 508-515.
Blair, L. J., Nordhues, B. A., Hill, S. E., Scaglione, K. M., O'Leary, J. C., 3rd, Fontaine, S. N., Breydo, L., Zhang, B., Li, P., Wang, L., et aL (2013). Accelerated neurodegeneration through chaperone-mediated oligomerization of tau. J Clin Invest 123, 4158-4169.
Chae, J.-I., Kim, D.-W., Lee, N., Jeon, Y.-J., Jeon, I., Kwon, J., Kim, J., Soh, Y., Lee, D.-S., Seo, Kang S., et aL (2012). Quantitative proteomic analysis of induced pluripotent stem cells derived from a human Huntington's disease patient. Biochemical Journal 446, 359-371.
Cheng, P. N., Liu, C., Zhao, M., Eisenberg, D., and Nowick, J. S. (2012). Amyloid beta-sheet mimics that antagonize protein aggregation and reduce amyloid toxicity. Nat Chem 4, 927-933.
David, D. C., Layfield, R., Serpell, L., Narain, Y., Goedert, M., and Spillantini, M. G. (2002). Proteasomal degradation of tau protein. J Neurochem 83, 176-185.
DiFiglia, M., Sapp, E., Chase, K. O., Davies, S. W., Bates, G. P., Vonsattel, J. P., and Aronin, N. (1997). Aggregation of Huntingtin in Neuronal Intranuclear Inclusions and Dystrophic Neurites in Brain. Science 277, 1990-1993.
Duyao, M. P., Auerbach, A. B., Ryan, A., Persichetti, F., Barnes, G. T., McNeil, S. M., Ge, P., Vonsattel, J. P., Gusella, J. F., Joyner, A. L., et aL (1995). Inactivation of the mouse Huntington's disease gene homolog Hdh. Science 269, 407-410.
Ebert, A. D., Shelley, B. C., Hurley, A. M., Onorati, M., Castiglioni, V., Patitucci, T. N., Svendsen, S. P., Mattis, V. B., McGivern, J. V., Schwab, A. J., et aL (2013). EZ spheres: a stable and expandable culture system for the generation of pre-rosette multipotent stem cells from human ESCs and iPSCs. Stem Cell Res 10, 417-427.
Eichinger, L., Pachebat, J. A., Glockner, G., Rajandream, M. A., Sucgang, R., Berriman, M., Song, J., Olsen, R., Szafranski, K., Xu, Q., et al. (2005). The genome of the social amoeba Dictyostelium discoideum. Nature 435, 43-57.
Eskici, G., and Gur, M. (2013). Computational design of new Peptide inhibitors for amyloid Beta (abeta) aggregation in Alzheimer's disease: application of a novel methodology. PLoS One 8, e66178.
Grune, T., Botzen, D., Engels, M., Voss, P., Kaiser, B., Jung, T., Grimm, S., Ermak, G., and Davies, K. J. (2010). Tau protein degradation is catalyzed by the ATP/ubiquitin-independent 20S proteasome under normal cell conditions. Arch Biochem Biophys 500, 181-188.
Hocking, T. D. (2017). directlabels: Direct Labels for Multicolor Plots. R package version 20170331
Hoffner, G., and Djian, P. (2014). Monomeric, oligomeric and polymeric proteins in huntington disease and other diseases of polyglutamine expansion. Brain Sci 4, 91-122.
Holmberg, C. I., Staniszewski, K. E., Mensah, K. N., Matouschek, A., and Morimoto, R. I. (2004). Inefficient degradation of truncated polyglutamine proteins by the proteasome. EMBO J 23, 4307-4318.
Kakkar, V., Mansson, C., de Mattos, E. P., Bergink, S., van der Zwaag, M., van Waarde, M. A., Kloosterhuis, N. J., Melki, R., van Cruchten, R. T., Al-Karadaghi, S., et al. (2016). The S/T-Rich Motif in the DNAJB6 Chaperone Delays Polyglutamine Aggregation and the Onset of Disease in a Mouse Model. Mol Cell.
Kaye, J. A., and Finkbeiner, S. (2013). Modeling Huntington's disease with induced pluripotent stem cells. Molecular and Cellular Neuroscience 56, 50-64.
Kazemi-Esfarjani, P., and Benzer, S. (2000). Genetic suppression of polyglutamine toxicity in Drosophila. Science 287, 1837-1840.
Knecht, D., and Pang, K. M. (1995). Electroporation of Dictyostelium discoideum. Methods in molecular biology (Clifton, N. J.) 47, 321-330.
Koyuncu, S., Fatima, A., Gutierrez-Garcia, R., and Vilchez, D. (2017). Proteostasis of Huntingtin in Health and Disease. Int J Mol Sci 18.
Kuiper, E. F., de Mattos, E. P., Jardim, L. B., Kampinga, H. H., and Bergink, S. (2017). Chaperones in Polyglutamine Aggregation: Beyond the Q-Stretch. Front Neurosci 11, 145.
Kuspa, A. (2006). Restriction enzyme-mediated integration (REMI) mutagenesis. Methods in molecular biology (Clifton, N. J.) 346, 201-209.
Kwon, Y. T., and Ciechanover, A. (2017). The Ubiquitin Code in the Ubiquitin-Proteasome System and Autophagy. Trends Biochem Sci 42, 873-886.
Li, H., Li, S.-H., Yu, Z.-X., Shelbourne, P., and Li, X.-J. (2001). Huntingtin Aggregate-Associated Axonal Degeneration is an Early Pathological Event in Huntington's Disease Mice. The Journal of Neuroscience 21, 8473-8481.
Malinovska, L., Palm, S., Gibson, K., Verbavatz, J. M., and Alberti, S. (2015). Dictyostelium discoideum has a highly Q/N-rich proteome and shows an unusual resilience to protein aggregation. Proc Natl Acad Sci USA 112, E2620-2629.
McGivern, J. V., Patitucci, T. N., Nord, J. A., Barabas, M. A., Stucky, C. L., and Ebert, A. D. (2013). Spinal muscular atrophy astrocytes exhibit abnormal calcium regulation and reduced growth factor production. Glia 61, 1418-1428.
Meriin, A. B., Zhang, X., He, X., Newnam, G. P., Chernoff, Y. O., and Sherman, M. Y. (2002). Huntington toxicity in yeast model depends on polyglutamine aggregation mediated by a prion-like protein Rnq1. The Journal of cell biology 157, 997-1004.
Michalik, A., and Van Broeckhoven, C. (2004). Proteasome degrades soluble expanded polyglutamine completely and efficiently. Neurobiol Dis 16, 202-211.
Miller, V. M., Nelson, R. F., Gouvion, C. M., Williams, A., Rodriguez-Lebron, E., Harper, S. Q., Davidson, B. L., Rebagliati, M. R., and Paulson, H. L. (2005). CHIP suppresses polyglutamine aggregation and toxicity in vitro and in vivo. J Neurosci 25, 9152-9161.
Nagai, Y., Inui, T., Popiel, H. A., Fujikake, N., Hasegawa, K., Urade, Y., Goto, Y., Naiki, H., and Toda, T. (2007). A toxic monomeric conformer of the polyglutamine protein. Nat Struct Mol Biol 14, 332-340.
Nasir, J., Floresco, S. B., O'Kusky, J. R., Diewert, V. M., Richman, J. M., Zeisler, J., Borowski, A., Marth, J. D., Phillips, A. G., and Hayden, M. R. (1995). Targeted disruption of the Huntington's disease gene results in embryonic lethality and behavioral and morphological changes in heterozygotes. Cell 81, 811-823.
Nath, S. R., and Lieberman, A. P. (2017). The Ubiquitination, Disaggregation and Proteasomal Degradation Machineries in Polyglutamine Disease. Front Mol Neurosci 10, 78.
Paulson, H. L., Bonini, N. M., and Roth, K. A. (2000). Polyglutamine disease and neuronal cell death. Proc Natl Acad Sci USA 97, 12957-12958.
Pei, J., Kim, B. H., and Grishin, N. V. (2008). PROMALS3D: a tool for multiple protein sequence and structure alignments. Nucleic Acids Res 36, 2295-2300.
Pilcher, K. E., Fey, P., Gaudet, P., Kowal, A. S., and Chisholm, R. L. (2007). A reliable general purpose method for extracting genomic DNA from Dictyostelium cells. Nat Protocols 2, 1325-1328.
Qin, Z. H., Wang, Y., Kegel, K. B., Kazantsev, A., Apostol, B. L., Thompson, L. M., Yoder, J., Aronin, N., and DiFiglia, M. (2003). Autophagy regulates the processing of amino terminal huntingtin fragments. Hum Mol Genet 12, 3231-3244.
Ravikumar, B., Duden, R., and Rubinsztein, D. C. (2002). Aggregate-prone proteins with polyglutamine and polyalanine expansions are degraded by autophagy. Hum Mol Genet 11, 1107-1117.
Sambataro, F., and Pennuto, M. (2017). Post-translational Modifications and Protein Quality Control in Motor Neuron and Polyglutamine Diseases. Front Mol Neurosci 10, 82.
Santarriaga, S., Petersen, A., Ndukwe, K., Brandt, A., Gerges, N., Bruns Scaglione, J., and Scaglione, K. M. (2015). The Social Amoeba Dictyostelium discoideum Is Highly Resistant to Polyglutamine Aggregation. J Biol Chem 290, 25571-25578.
Sato, T., Kienlen-Campard, P., Ahmed, M., Liu, W., Li, H., Elliott, J. I., Aimoto, S., Constantinescu, S. N., Octave, J. N., and Smith, S. O. (2006). Inhibitors of amyloid toxicity based on beta-sheet packing of Abeta40 and Abeta42. Biochemistry 45, 5503-5516.
Satyal, S. H., Schmidt, E., Kitagawa, K., Sondheimer, N., Lindquist, S., Kramer, J. M., and Morimoto, R. I. (2000). Polyglutamine aggregates alter protein folding homeostasis in Caenorhabditis elegans. Proc Natl Acad Sci USA 97, 5750-5755.
Scherzinger, E., Lurz, R., Turmaine, M., Mangiarini, L., Hollenbach, B., Hasenbank, R., Bates, G. P., Davies, S. W., Lehrach, H., and Wanker, E. E. (1997). Huntingtin-encoded polyglutamine expansions form amyloid-like protein aggregates in vitro and in vivo. Cell 90, 549-558.
Scherzinger, E., Sittler, A., Schweiger, K., Heiser, V., Lurz, R., Hasenbank, R., Bates, G. P., Lehrach, H., and Wanker, E. E. (1999a). Self-assembly of polyglutamine-containing huntingtin fragments into amyloid-like fibrils: implications for Huntington's disease pathology. Proc Natl Acad Sci USA 96, 4604-4609.
Scherzinger, E., Sittler, A., Schweiger, K., Heiser, V., Lurz, R., Hasenbank, R., Bates, G. P., Lehrach, H., and Wanker, E. E. (1999b). Self-assembly of polyglutamine-containing huntingtin fragments into amyloid-like fibrils: Implications for Huntington's disease pathology. Proceedings of the National Academy of Sciences 96, 4604-4609.
Schwab, A. J., Sison, S. L., Meade, M. R., Broniowska, K. A., Corbett, J. A., and Ebert, A. D. (2017). Decreased Sirtuin Deacetylase Activity in LRRK2 G2019S iPSC-Derived Dopaminergic Neurons. Stem cell reports 9, 1839-1852.
Team, R. C. (2016). R: A language and environment for statical computing. R Foundation for Statistical Computing.
The Hd iPsc Consortium (2012). Induced Pluripotent Stem Cells from Patients with Huntington's Disease Show CAG-Repeat-Expansion-Associated Phenotypes. Cell Stem Cell 11, 264-278.
Tomioka, I., Ishibashi, H., Minakawa, E. N., Motohashi, H. H., Takayama, O., Saito, Y., Popiel, H. A., Puentes, S., Owari, K., Nakatani, T., et al. (2017). Transgenic Monkey Model of the Polyglutamine Diseases Recapitulating Progressive Neurological Symptoms. eNeuro 4.
Tsigelny, I. F., Crews, L., Desplats, P., Shaked, G. M., Sharikov, Y., Mizuno, H., Spencer, B., Rockenstein, E., Trejo, M., Platoshyn, O., et al. (2008). Mechanisms of hybrid oligomer formation in the pathogenesis of combined Alzheimer's and Parkinson's diseases. PLoS One 3, e3135.
Vicente, J. J., Galardi-Castilla, M., Escalante, R., and Sastre, L. (2008). Structural and functional studies of a family of Dictyostelium discoideum developmentally regulated, prestalk genes coding for small proteins. BMC Microbiol 8, 1.
Waelter, S., Boeddrich, A., Lurz, R., Scherzinger, E., Lueder, G., Lehrach, H., and Wanker, E. E. (2001). Accumulation of mutant huntingtin fragments in aggresome-like inclusion bodies as a result of insufficient protein degradation. Mol Biol Cell 12, 1393-1407.
Wanker, E. E., Scherzinger, E., Heiser, V., Sittler, A., Eickhoff, H., and Lehrach, H. (1999). Membrane filter assay for detection of amyloid-like polyglutamine-containing protein aggregates. Methods in enzymology 309, 375-386.
Wickham, H. (2016). scales: Scale Functions for Visualization. R package Version 041 https://CRAN,R-project.org/package=scales.
Wickham, H. (2017). tidyverse: Easily Install and Load ‘Tidyverse’ Packages. R package version 111
Wiegand, S., Kruse, J., Gronemann, S., and Hammann, C. (2011). Efficient generation of gene knockout plasmids for Dictyostelium discoideum using one-step cloning. Genomics 97, 321-325.
Williams, A. J., and Paulson, H. L. (2008). Polyglutamine neurodegeneration: protein misfolding revisited. Trends in neurosciences 31, 521-528.
Zeitlin, S., Liu, J. P., Chapman, D. L., Papaioannou, V. E., and Efstratiadis, A. (1995). Increased apoptosis and early embryonic lethality in mice nullizygous for the Huntington's disease gene homologue. Nat Genet 11, 155-163.

### Example 2: SRCP1 Reduces the Levels of Protein Aggregates for an ALS Associated Neurodegenerative Disease Proteins

This example demonstrates that SRCP1 can reduce protein aggregation of SOD-1 associated with familial ALS. In ALS, both the upper motor neurons and the lower motor neurons degenerate or die, and stop sending messages to the muscles. Unable to function, the muscles gradually weaken, start to twitch (called fasciculations), and waste away (atrophy). At some point, the brain loses its ability to initiate and control voluntary movements.

A hallmark of ALS is the abnormal accumulation of protein aggregates (or deposits) containing the mutated SOD1.

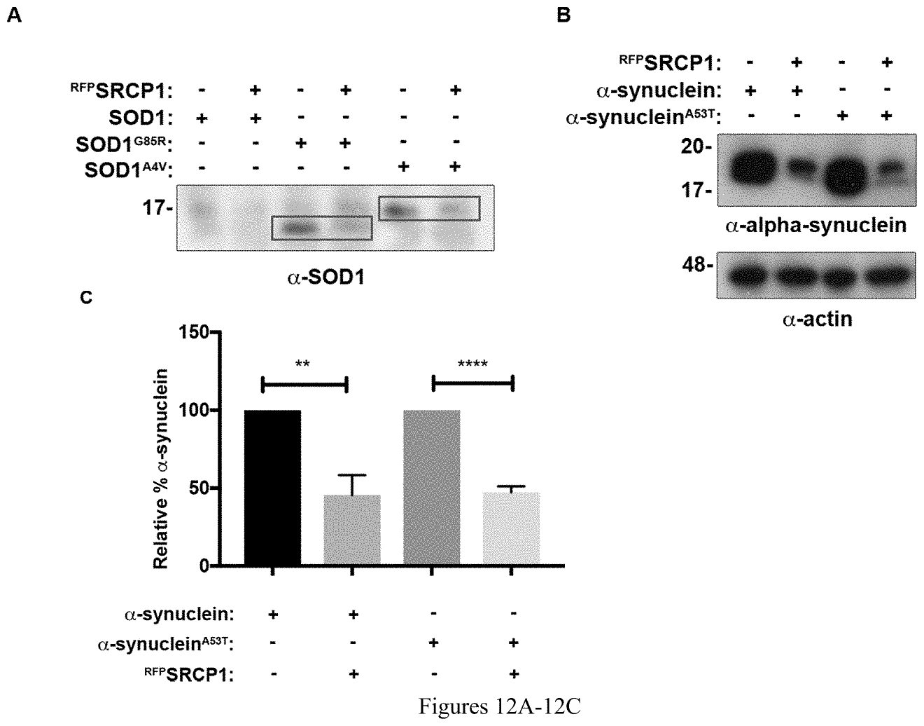

This Example demonstrates that the overexpression of SRCP1 reduces the levels of aggregated mutant SOD1 in HEK293 cells FIG. 12A). HEK293 cells were transfected with either wild-type or mutant (G85R or A4V) SOD1 either in the presence or absence of SRCP1 for 48 hours. Cells were then collected and lysed prior to ultracentrifugation to isolate aggregated proteins. After ultracentrifugation cell protein in the pellet was suspended in Laemmli buffer and analyzed by SDS-PAGE and western blot

### Example 3: SRCP1 Reduces the Levels of Protein Aggregates for a Parkinson's Associated Neurodegenerative Disease Proteins

This example demonstrates the use of the SRCP1 proteins described herein can be used to reduce the aggregation of α-Synuclein in diseases associated with aggregation of α-Synuclein, including synucleinopathies and Parkinson's disease. α-Synuclein is encoded by the SNCA gene and has been linked genetically and neuropathologically to PD. α-Synuclein is thought to contribute to PD pathogenesis in at least one way by aberrant soluble oligomeric conformations of α-Synuclein (called protofibrils) that are the toxic to cells and mediate disruption of cellular homeostasis and neuronal death by having an effect on various intracellular targets, including synaptic function. α-Synuclein is also dysregulated in other neurodegenerative conditions, termed synucleinopathies.

As demonstrated in FIG. 12B, overexpression SRCP1 in HEK293 cells results in a reduction of α-synuclein. HEK 293 cells were transfected with either wild-type or mutant α-synuclein in the presence or absence of SRCP1. Cells were collected 48 hours later and analyzed by SDS-PAGE and western blot. This Example demonstrates that SRCP1 can further inhibit protein aggregation in another neurodegenerative disease.

Each publication, patent, and patent publication cited in this disclosure is incorporated in reference herein in its entirety. The present invention is not intended to be limited to the foregoing examples, but encompasses all such modifications and variations as come within the scope of the appended claims.

### SEQUENCE LISTING STATEMENT

The application includes the sequence listing below and filed in computer readable format (TXT), which is incorporated by reference into this application in its entirety.

SEQ ID NO: 1-SRCP1 gene (NC_007092):
atgacaattt tatgtaagta aaatatttaa tgattaaatt
taatagtttt caaattagaa tttatttatt tattaacatt
tttttttttt tttttttttt tttttttaaa attaaatttc
actttatttt aaaaagcaac ctttacatca ttttcaaatc
cacccaaatt aaataaatct tcattttcat catcaacggg
ttcatcatta tcaatgggat caaattcatt tgcatggggg
ggaggttggg ggggttttgg gggcccaaaa gggggaagtt
ttaatgtgga cattgctggaaatttaattt ggggggttta
ttagggggtt tataaggggg ggagtgggac tggttaagtg
gagagggctc caaaagggat gcaagcagcg cctga

NC_007092.3:2790501-2790895 Dictyostelium discoideum AX4 chromosome 6 chromosome, whole genome shotgun sequence

ATGACAATTTTATGTAAGTAAAATATTTAATGATTAAATTTAATAGT
TTTCAAATTAGAATTTATTTATTTATTAACATTTTTTTTTTTTTTTT
TTTTTTTTTTTTTAAAATTAAATTTCACTTTATTTTAAAAAGCAACC
TTTACATCATTTTCAAATCCACCCAAATTAAATAAATCTTCATTTTC
ATCATCAACGGGTTCATCATTATCAATGGGATCAAATTCATTTGCAT
GGGGGGGAGGTTGGGGGGGTTTTGGGGGCCCAAAAGGGGGAAGTTTT
AATGTGGACATTGCTGGAAATTTAATTTGGGGGGTTTATTAGGGGGT
TTATAAGGGGGGGAGTGGGACTGGTTAAGTGGAGAGGGCTCCAAAAG
GGATGCAAGCAGCGCCTGA
SEQ ID NO: 2 SRCP1 protein
(DDB0252803|DDB_G0293362|Protein|gene:
DDB_G0293362 on chromosome: 6 position 2790501
to 2790895)
MTILSTFTSFSNPPKLNKSSFSSSTGSSLSMGSNSFAWGGGWGGFGG
PKGGSFNVDIAGNLIWGVYGFIRGGVGLVKWRGLQKGCKQP
SEQ ID NO: 3: SRCP1 61-70 mutant (non-functional):
MTILSTFTSFSNPPKLNKSSFSSSTGSSLSMGSNSFAWGGGWGGFGG
PKGGSFNVDIAGNAAAAAAAAAAGGVGLVKWRGLQKGCKQP
SEQ ID NO: 4 SRCP1 71-80 mutant (this one was
functional):
MTILSTFTSFSNPPKLNKSSFSSSTGSSLSMGSNSFAWGGGWGGFGG
PKGGSFNVDIAGNLIWGVYGFIRAAAAAAAAAALQKGCKQP
SEQ ID NO: 5 SRCP1 AA 61-70 fragment:
LIWGVYGFIR
SEQ ID NO: 6 STA mutation in SRCP1 (mutation
within 1-37 of Thr or Ser to Ala):
MAILAAFAAF ANPPKLNKAA FAAAAGAALA MGANAFAW
GGGWGGFGGPKGGSFNVDIAGNLIWGVYGFIRGGVGLVKWRGLQKGC
KQP
SEQ ID NO: 7 SRCP1 fragment 71-80:
GGVGLVKWRG
SEQ ID NO: 8 SRCP1 1-37 fragment:
MTILSTFTSF SNPPKLNKSS FSSSTGSSLS MGSNSFAW
SEQ ID NO: 9 SRCP1 peptide 61-80
LIWGVYGFIRGGVGLVKWRG
SEQ ID NO: 10 SRCP1 peptide
CLIWGVYGFIRGGVGLVKWRGC
(note: disulfide bridge between two cys)

We claim:

1. An isolated peptide inhibitor of protein aggregation comprising SEQ ID NO:5 (LIWGVYGFIR).

2. The isolated peptide of claim 1, wherein the peptide is SEQ ID NO:5.

3. The isolated peptide of claim 1, wherein the peptide comprises SEQ ID NO:9 (LIWGVYGFIRGGVGLVKWRG).

4. The isolated peptide of claim 3, wherein the peptide is SEQ ID NO:9.

5. The isolated peptide of claim 3, wherein the peptide further comprises additional amino acids selected from amino acids 1-60 or 71-88 of SEQ ID NO:2.

6. The isolated peptide of claim 3, wherein the peptide further comprises additional amino acids selected from amino acids 1-60 or 71-88 of SEQ ID NO:2 with at least one amino acid mutation within amino acids 1-60 or 71-88.

7. The isolated peptide of claim 6, wherein the isolated peptide comprises at least 3 amino acid mutations.

8. The isolated peptide of claim 6 or 7, wherein the peptide comprises at least one substitution of a threonine or a serine to an alanine within amino acids 1-38 of SEQ ID NO:2 or amino acids 71-88 of SEQ ID NO:2.

9. The isolated peptide of any one of claims 6-8, wherein the peptide comprises at least two or more substitutions of a threonine or a serine to alanine within amino acids 1-38 of SEQ ID NO:2 or amino acids 71-88 of SEQ ID NO:2.

10. The isolated peptide of any one of claims 6-9, wherein the peptide comprises from 1 to 16 of the threonine or serine substituted with an alanine in amino acids 1-38.

11. The isolated peptide of any one of claims 6-10, wherein all threonine or serine within amino acids 1-38 of SEQ ID NO: 1 are substituted with alanine.

12. The isolated peptide of any one of claims 6-11, wherein the peptide comprises SEQ ID NO:6.

13. The isolated peptide of any one of claims 6-11, wherein the peptide comprises substitution of all of the amino acids of 71-80 of SEQ ID NO:2 with alanine.

14. The isolated peptide of claim 13, wherein the peptide comprises SEQ ID NO:4.

15. The isolated peptide of any one of the preceding claims, wherein the peptide has at least 40% sequence similarity with SEQ ID NO:2 and wherein amino acids 61-70 are SEQ ID NO:5.

16. The isolated peptide of claim 15, wherein the peptide has at least 60% sequence similarity with SEQ ID NO:2 and wherein amino acids 61-70 are SEQ ID NO:5.

17. The isolated peptide of claim 16, wherein the peptide has at least 80% sequence similarity with SEQ ID NO:2 and wherein amino acids 61-70 are SEQ ID NO:5.

18. The isolated peptide of any one of the preceding claims, wherein the peptide has at least 90% sequence similarity with SEQ ID NO:2 and wherein amino acids 61-70 are SEQ ID NO:5.

19. The isolated peptide of claim 1, wherein the peptide is SEQ ID NO: 10.

20. The isolated peptide of claim 18, wherein the peptide is SEQ ID NO:2.

21. The isolated peptide of any one of the preceding claims, wherein the isolated peptide is directly or indirectly linked to a tag or agent.

22. The isolated peptide of claim 21, wherein the tag is a peptide tag.

23. The isolated peptide of claim 22, wherein the tag is purification tag.

24. A vector able to express the isolated peptide of any one of claims 1-23.

25. A vector comprising (a) a polynucleotide sequence encoding the isolated peptide of any one of claims 1-23 and (b) a heterologous polynucleotide sequence.

26. The vector of claim 25, wherein the heterologous polynucleotide sequence comprises a tag or agent.

27. The vector of any one of claims 25-26, wherein the vector is a DNA expression vector.

28. The vector of any one of claims 24-25, wherein the vector is a plasmid.

29. The vector of any one of claims 24-27, wherein the vector is a viral vector.

30. The vector of claim 29, wherein the heterologous nucleic acid sequence is a viral sequence.

31. The vector of claim 29 or 30, wherein the vector is a retrovirus vector or an adeno-associated virus vector.

32. A virus produced by the vector of any one of claims 29-31.

33. A host cell comprising the vector of any one of claims 24-31, wherein the host cell expresses the isolated peptide.

34. A pharmaceutical composition comprising the isolated peptide inhibitor of protein aggregation of any one of claims 1-22 and a pharmaceutically acceptable carrier.

35. The pharmaceutical composition of claim 33, wherein the pharmaceutically acceptable carrier is a buffered solution.

36. A method of reducing or inhibiting protein aggregation in a host cell, the method comprising:
(a) introducing within the host cell an effective amount of
(i) the isolated peptide of any one of claims 1-22,
(ii) the vector of any one of claims 24-31,
(iii) the virus of claim 32; or
(iv) the composition of claim 34 or 35, wherein the protein aggregation within the host cell is reduced or inhibited.

37. The method of claim 36, wherein the protein aggregation is polyglutamine protein aggregation.

38. The method of claim 36 or 37, wherein the host cell is a human cell.

39. The method of claim 38, wherein the human cell is in vivo.

40. The method of any one of claims 36-39, wherein the protein aggregation is reduced by at least 40% within the host cell.

41. The method of claim 40, wherein the protein aggregation is reduced by at least 60% within the host cell.

42. The method of any one of claims 36-41, wherein the protein is huntingtin.

43. The method of any one of claims 36-41, wherein the protein is SOD-1.

44. The method of any one of claims 36-41, wherein the protein is α-synuclein.

45. A method of treating a polyglutamine disease in a subject in need thereof, the method comprising the steps of:
(a) administering to the subject one of the following
(i) the isolated peptide of any one of claims 1-22,
(ii) the vector of any one of claims 24-31,
(iii) the virus of claim 32; or
(iv) the composition of claim 34 or 35, in an effective amount to reduce, inhibit or prevent at least one symptom of the polyglutamine disease associated with aggregation of the polyglutamine protein.

46. The method of claim 45, wherein the polyglutamine disease is selected from the group consisting of SBMA; Huntington's disease; spinocerebellar ataxia type 1, spinocerebellar ataxia type 2, spinocerebellar ataxia type 3, spinocerebellar ataxia type 6, spinocerebellar ataxia type 7, spinocerebellar ataxia type 17; and dentatorubral-pallidoluysian atrophy (DRPLA).

47. The method of claim 46, wherein the polyglutamine disease is Huntington's disease.

48. The method of claim 45-47, wherein the subject is a mammal.

49. The method of claim 48, wherein the subject is a human.

50. A method of treating a disease associated with aggregation of a protein in a subject in need thereof, the method comprising the steps of:
(a) administering to the subject
(i) the isolated peptide of any one of claims 1-22,
(ii) the vector of any one of claims 24-31,
(iii) the virus of claim 32; or
(iv) the composition of claim 34 or 35,
in an effective amount to reduce, inhibit or prevent at least one symptom of the disease associated with aggregation.

51. The method of claim 50, wherein the disease is Parkinson's disease.

52. The method of claim 50, wherein the disease is amyotrophic lateral sclerosis (ALS).

53. The method of any one of claims 50-52, wherein the subject is a human.
